(19) United States
(12) Patent Application Publication
(10) Pub. No.: US 2022/0257592 A1
(43) Pub. Date: Aug. 18, 2022

(54) METHODS OF TREATING CANCER

(71) Applicants: HOFFMANN-LA ROCHE INC., Little Falls, NJ (US); IGNYTA, INC., South San Francisco, CA (US); GENENTECH, INC., South San Francisco, CA (US)

(72) Inventors: Georgina Meneses-Lorente, Welwyn Garden City (GB); Edna Chow Maneval, Del Mar, CA (US); Pratik S. Multani, San Diego, CA (US); Susan Diane Eng, San Francisco, CA (US); Sergio Rene Ley Acosta, Danville, CA (US)

(73) Assignees: HOFFMANN-LA ROCHE INC., LITTLE FALLS, NJ (US); IGNYTA, INC., South San Francisco, CA (US); GENENTECH, INC., South San Francisco, CA (US)

(21) Appl. No.: 17/590,603

(22) Filed: Feb. 1, 2022

Related U.S. Application Data
(63) Continuation of application No. PCT/US2020/046229, filed on Aug. 13, 2020.
(60) Provisional application No. 62/886,948, filed on Aug. 14, 2019.

(51) Int. Cl. A61K 31/496; A61P 35/00

(52) U.S. Cl. CPC A61K 31/496; A61P 35/00

(57) ABSTRACT

Provided herein are methods of treating cancer in a patient in need thereof includes administering to the patient a therapeutically effective amount of entrectinib wherein the patient has impaired hepatic function and methods of managing adverse reactions of administration of entrectinib in a patient.

### CROSS-REFERENCE TO RELATED APPLICATIONS

This application is a continuation of International Patent Application No. PCT/US2020/046229, filed Aug. 13, 2020, which claims the benefit of U.S. Provisional Patent Application No. 62/886,948, filed Aug. 14, 2019, both of which are incorporated by reference herein in their entirety.

### FIELD

The present disclosure relates to methods of treating cancer in patients with impaired hepatic function utilizing pharmaceutical compositions disclosed herein. The present disclosure also relates to methods of managing adverse reactions of administration of entrectinib in patients.

### SUMMARY

In one aspect, provided herein are methods of treating cancer in a patient in need thereof, the methods comprising administering to the patient a therapeutically effective amount of entrectinib wherein the patient has impaired hepatic function. In some embodiments, the impaired hepatic function is mild hepatic dysfunction, as assessed by Child-Pugh scoring system. In some embodiments, the impaired hepatic function is moderate hepatic dysfunction, as assessed by Child-Pugh scoring system. In some embodiments, the impaired hepatic function is severe hepatic dysfunction, as assessed by Child-Pugh scoring system. In some embodiments, the Child-Pugh scoring system comprises assessment of encephalopathy grade, ascites, serum bilirubin concentration, serum albumin concentration, and prothrombin time. In some embodiments, the mild, moderate, or severe hepatic dysfunction arises from liver cirrhosis. In some embodiments, the liver cirrhosis is a result of parenchymal liver disease. In some embodiments, the impaired hepatic function is mild, moderate, or severe hepatic impairment as assessed by National Cancer Institute organ dysfunction working group system. In some embodiments, the National Cancer Institute organ dysfunction working group (NCI-ODWG) system measures total bilirubin concentration and aspartate aminotransferase concentration. In some embodiments, the patient has a total bilirubin concentration that is about greater than 1.5 times the upper limit of normal to about 3 times the upper limit of normal. In some embodiments, the patient has a total bilirubin concentration that is greater than 1.5 times the upper limit of normal to about 3 times the upper limit of normal. In some embodiments, the therapeutically effective amount is 600 mg or less. In some embodiments, the therapeutically effective amount is 100 mg, 200 mg, 300 mg, 400 mg, or 500 mg.

In another aspect, provided herein is a method of treating cancer or managing adverse reactions of administration of entrectinib in a patient in need thereof, entrectinib for use in the treatment of cancer, or a use of entrectinib in the manufacture of a medicament for the treatment of cancer, comprising:
prior to administering entrectinib to the patient,
   (i) optionally advising the patient of a risk of one or more adverse reactions from the administration of entrectinib; and/or
   (ii) optionally assessing one or more parameters in the patient associated with an adverse reaction (e.g., LVEF for CHF, serum uric acid levels for hyperuricemia, QT interval and electrolytes for QT interval prolongation);
initiating administration of entrectinib to the patient with an initial dose (e.g., 600 mg orally per day);
monitoring the patient for adverse reaction; and
continuing to administer entrectinib to the patient in the absence of any adverse reaction or the absence of at least Grade 2 severity of adverse reaction in the patient or until cancer progression; or
if the patient experiences Grade 3 or Grade 4 severity of the adverse reaction, withholding entrectinib and resuming administration of entrectinib at the initial dose or at a reduced dose upon patient recovery to Grade 2 or less severity of the adverse reaction.

In another aspect, provided herein is a method of treating cancer or managing adverse reactions of administration of entrectinib in a patient in need thereof, entrectinib for use in the treatment of cancer, or a use of entrectinib in the manufacture of a medicament for the treatment of cancer, comprising:
prior to administering entrectinib to the patient,
   (i) optionally advising the patient of a risk of one or more adverse reactions from the administration of entrectinib; and/or
   (ii) optionally assessing one or more parameters in the patient associated with an adverse reaction (e.g., LVEF for CHF, serum uric acid levels for hyperuricemia, QT interval and electrolytes for QT interval prolongation);
initiating administration of entrectinib to the patient with an initial dose (e.g., 600 mg orally per day);
monitoring the patient for adverse reaction; and
if the patient does not exhibit a severe adverse reaction, continuing to administer entrectinib until cancer progression; or
if the patient experiences a severe adverse reaction, withholding entrectinib and, upon patient recovery, resuming administration of entrectinib at the initial dose or at a reduced dose wherein patient recovery is either the absence of or a reduction of the severe adverse reaction or if the severe adverse reaction does not resolve within 4 weeks or if the severe adverse reaction recurs, permanently discontinuing administration of entrectinib.

In some embodiments, the method is of treating cancer in a patient in need thereof. In some embodiments, the method is of managing adverse reactions of administration of entrectinib in a patient. In some embodiments, the adverse reaction is a cardiac disorder (e.g., congestive heart failure and QT interval prolongation), a central nervous system (CNS) adverse reaction, fracture (e.g., skeletal fracture), hepatotoxicity, hyperuricemia, a vision disorder, anemia/neutropenia, a cognitive disorder, transaminase elevation, an embryo-fetal toxicity, syncope, or an interstitial lung disease. In some embodiments, the adverse reaction is congestive heart failure, a central nervous system (CNS) adverse reaction, hepatotoxicity, hyperuricemia, QT interval prolongation, a vision disorder, or anemia/neutropenia. In some embodiments, the adverse reaction is congestive heart failure, a cognitive disorder, hyperuricemia, QT interval prolongation, a transaminase elevation, or anemia/neutropenia. In some embodiments, the adverse reaction is anemia/neutropenia, a central nervous system (CNS) adverse reaction, hepatotoxicity, hyperuricemia, syncope, congestive heart failure, QT interval prolongation, or a vision disorder. In some embodiments, the adverse reaction is cardiac disorder (except QT interval prolongation), QT interval prolongation, a cognitive disorder or ataxia, syncope, anemia/neutropenia, or an interstitial lung disease. In some embodiments, the adverse reaction is anemia/neutropenia, a cognitive disorder, congestive heart failure, or QT interval prolongation.

In another aspect, provided herein are methods of treating cancer in a patient in need thereof, the methods comprising:
assessing left ventricular ejection fraction (LVEF) of the patient prior to administering entrectinib to the patient;
initiating administration of entrectinib to the patient;
monitoring the patient for clinical signs and symptoms of congestive heart failure (CHF), including shortness of breath and edema;
optionally obtaining a MRI or cardiac biopsy to make a diagnosis of CHF; and
continuing to administer entrectinib to the patient in the absence of adverse reaction in the patient or until cancer progression; or
if the patient experiences Grade 2 or 3 severity of CHF, withholding administration of entrectinib, instituting appropriate medical management, reassessing LVEF and severity of CHF, and resuming administration of entrectinib at a reduced dose upon patient recovery to less than or equal to Grade 1 severity of CHF or permanently discontinuing administration of entrectinib if the patient experiences Grade 4 severity of CHF.

In another aspect, provided herein are methods of treating cancer in a patient in need thereof, the methods comprising:
prior to administration of entrectinib to the patient,
   advising the patient of a risk of one or more central nervous system (CNS) adverse reactions from the administration of entrectinib; and
   advising the patient not to drive or operate hazardous machinery if the patient experiences one or more CNS adverse reactions from the administration of entrectinib;
   initiating administration of entrectinib to the patient with an initial dose;
   monitoring the patient for one or more CNS adverse reactions; and
   continuing to administer entrectinib to the patient in the absence of adverse reaction in the patient or until cancer progression; or
   if the patient experiences Grade 2 severity of CNS adverse reaction, withholding administration of entrectinib and resuming administration of entrectinib at the initial dose or at a reduced dose upon patient recovery to less than or equal to Grade 1 severity of CNS adverse reaction; or
   if the patient experiences Grade 3 severity of CNS adverse reaction, withholding administration of entrectinib and resuming administration of entrectinib at the reduced dose upon patient recovery to less than or equal to Grade 1 severity of CNS adverse reaction; or
   if the patient experiences Grade 4 severity of CNS adverse reaction, permanently discontinuing administration of entrectinib.

In another aspect, provided herein are methods of treating cancer in a patient in need thereof, the methods comprising:
initiating administration of entrectinib to the patient (e.g., at an initial dose);
   monitoring the patient for one or more signs or symptoms of skeletal fracture;
   if the patient exhibits one or more signs or symptoms of skeletal fracture, promptly evaluating the patient and instituting appropriate medical management; and
   continuing to administer entrectinib to the patient in the absence of additional adverse reaction in the patient or until cancer progression.

In another aspect, provided herein are methods of treating cancer in a patient in need thereof, the methods comprising:
initiating administration of entrectinib to the patient with an initial dose;
   monitoring the patient with liver tests, including ALT and AST, every 2 weeks during the first month of administration, then monthly thereafter, and as clinically indicated; and
   continuing to administer entrectinib to the patient in the absence of adverse reaction in the patient or until cancer progression; or
   if the patient experiences Grade 3 severity of hepatotoxicity, withholding administration of entrectinib and resuming administration of entrectinib at the initial dose upon patient recovery within 4 weeks to less than or equal to Grade 1 severity of hepatotoxicity, or resuming administration of entrectinib at a reduced dose for recurrent Grade 3 severity of hepatotoxicity that resolve within 4 weeks; or permanently discontinuing administration of entrectinib if Grade 3 severity of hepatotoxicity does not resolve within 4 weeks; or
   if the patient experiences Grade 4 severity of hepatotoxicity, withholding administration of entrectinib and resuming administration of entrectinib at the reduced dose upon patient recovery within 4 weeks to less than or equal to Grade 1 severity of hepatotoxicity, or permanently discontinuing administration of entrectinib if Grade 4 severity of hepatotoxicity does not resolve within 4 weeks or recurs; or
   if the patient exhibits ALT or AST greater than 3 times the upper limit of normal (ULN) with concurrent total bilirubin greater than 1.5 times ULN in the absence of cholestasis or hemolysis, permanently discontinuing administration of entrectinib.

In another aspect, provided herein are methods of treating cancer in a patient in need thereof, the methods comprising:
assessing serum uric acid levels in the patient prior to initiating administration of entrectinib;
initiating administration of entrectinib to the patient with an initial dose;
reassessing serum uric acid levels periodically during administration of entrectinib and monitoring the patient for signs and symptoms of hyperuricemia; and
continuing to administer entrectinib to the patient in the absence of adverse reaction in the patient or until cancer progression; or
if the patient is symptomatic of hyperuricemia or experiences Grade 4 hyperuricemia, initiating treatment with urate-lowering medications as clinically indicated, withholding entrectinib and resuming administration of entrectinib at the initial dose or at a reduced dose upon improvement of signs or symptoms of hyperuricemia.

In another aspect, provided herein are methods of treating cancer in a patient in need thereof, the methods comprising:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc greater than 500 ms, withholding entrectinib until QTc interval recovers to baseline and resuming administration of entrectinib at the initial dose or at a reduced dose; or
if the patient exhibits Torsade de pointes, polymorphic ventricular tachycardia, or signs or symptoms of serious arrhythmia, permanently discontinuing administration of entrectinib.

In another aspect, provided herein are methods of treating cancer in a patient in need thereof, the methods comprising:
initiating administration of entrectinib to the patient with an initial dose;
monitoring the patient for a new vision disorder; and
continuing to administer entrectinib to the patient in the absence of adverse reaction in the patient or until cancer progression; or
if the patient experiences Grade 2 or higher severity of vision disorder, withholding entrectinib until improvement or stabilization of the vision disorder, and resuming administration of entrectinib at the initial dose or at a reduced dose.

In another aspect, provided herein are methods of treating cancer in a patient in need thereof, the methods comprising:
initiating administration of entrectinib to the patient with an initial dose;
monitoring the patient for anemia or neutropenia; and
continuing to administer entrectinib to the patient in the absence of adverse reaction in the patient or until cancer progression; or
if the patient experiences Grade 3 or Grade 4 severity of anemia or neutropenia, withholding entrectinib and resuming administration of entrectinib at the initial dose or at a reduced dose upon patient recovery to Grade 2 or less severity of anemia or neutropenia.

In another aspect, provided herein are methods of treating cancer in a patient in need thereof, the methods comprising:
prior to the administration of entrectinib to the patient,
   advising the patient of a potential for cognitive change from the administration of entrectinib; and
   advising the patient not to drive or operate hazardous machinery if the patient experiences one or more symptoms of cognitive change from the administration of entrectinib;
initiating administration of entrectinib to the patient with an initial dose;
monitoring the patient for a cognitive disorder; and
continuing to administer entrectinib to the patient in the absence of adverse reaction in the patient or until cancer progression; or
if the patient experiences an event of at least Grade 2 severity of cognitive disorder, withholding administration of entrectinib until patient recovery to less than or equal to Grade 1 severity of cognitive disorder and resuming administration of entrectinib at a reduced dose; or
if the patient experiences a recurrence of the event of at least Grade 2 severity of cognitive disorder, administering entrectinib at a further reduced dose; or
if the patient experiences prolonged, severe, or intolerable events of cognitive disorder, permanently discontinuing administration of entrectinib.

In another aspect, provided herein are methods of treating cancer in a patient in need thereof, the methods comprising:
initiating administration of entrectinib to the patient with an initial dose;
monitoring the patient for transaminase elevation; and
continuing to administer entrectinib to the patient in the absence of adverse reaction in the patient or until cancer progression; or
if the patient exhibits Grade 3 severity of transaminase elevation, withholding administration of entrectinib until patient recovery to less than or equal to Grade 1 severity of transaminase elevation; and resuming administration of entrectinib to the patient without change in dose upon patient recovery within 4 weeks to less than or equal to Grade 1 severity of transaminase elevation, or resuming administration of entrectinib at a reduced dose for recurrent Grade 3 severity of hepatotoxicity that resolve within 4 weeks, or permanently discontinuing administration of entrectinib to the patient if Grade 3 severity of transaminase elevation does not resolve within 4 weeks; or
if the patient exhibits Grade 4 severity of transaminase elevation, withholding administration of entrectinib until patient recovery to less than or equal to Grade 1 severity of transaminase elevation; and resuming administration of entrectinib to the patient without change in dose upon patient recovery within 4 weeks to less than or equal to Grade 1 severity of transaminase elevation, or permanently discontinuing administration of entrectinib to the patient if Grade 4 severity of transaminase elevation does not resolve within 4 weeks or recurs; or
if the patient exhibits ALT or AST elevation greater than 3 times the upper limit of normal

(ULN) with total bilirubin elevation being greater than 2 times ULN in the absence of cholestasis or hemolysis, permanently discontinuing administration of entrectinib to the patient.

In another aspect, provided herein are methods of treating cancer in a patient in need thereof, the methods comprising:
initiating administration of entrectinib to the patient with an initial dose;
monitoring the patient for congestive heart failure; and
continuing to administer entrectinib to the patient in the absence of adverse reaction in the patient or until cancer progression; or
if the patient exhibits Grade 2 or Grade 3 severity of congestive heart failure, withholding administration of entrectinib until patient recovery to less than or equal to Grade 1 severity of congestive heart failure; and resuming administration of entrectinib at a reduced dose; or
if the patient exhibits Grade 4 severity of congestive heart failure, withholding administration of entrectinib until patient recovery to less than or equal to Grade 1 severity of congestive heart failure; and resuming administration of entrectinib at a reduced dose or permanently discontinuing administration of entrectinib to the patient.

In another aspect, provided herein are methods of treating cancer in a patient in need thereof, the methods comprising:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc of 481 to 500 ms, withholding entrectinib until QTc interval recovers to baseline and resuming administration of entrectinib at the initial dose; or
if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or
if the patient exhibits Torsade de pointes, polymorphic ventricular tachycardia, or signs or symptoms of serious arrhythmia, permanently discontinuing administration of entrectinib.

In another aspect, provided herein are methods of treating cancer in a patient in need thereof, the methods comprising:
prior to the administration of entrectinib to the patient,
   advising the patient of a potential for embryo-fetal toxicity;
initiating administration of entrectinib to the patient (e.g., at an initial dose); and
continuing to administer entrectinib to the patient in the absence of adverse reaction in the patient or until cancer progression; or
if the patient becomes pregnant, permanently discontinuing administration of entrectinib.

In another aspect, provided herein are methods of treating cancer in a patient in need thereof, the methods comprising:
initiating administration of entrectinib to the patient with an initial dose;
monitoring the patient for adverse reaction; and
continuing to administer entrectinib to the patient in the absence of adverse reaction in the patient or until cancer progression; or
if the patient experiences Grade 3 or Grade 4 severity of adverse reaction, withholding entrectinib and resuming administration of entrectinib at the initial dose or at a reduced dose upon patient recovery to Grade 1 or less severity of adverse reaction or permanently discontinuing administration of entrectinib if adverse reaction does not resolve within 4 weeks or Grade 4 severity adverse reactions recur.

In another aspect, provided herein are methods of managing adverse reactions of administration of entrectinib in a patient, the methods comprising:
assessing left ventricular ejection fraction (LVEF) of the patient prior to administering entrectinib to the patient;
initiating administration of entrectinib to the patient;
monitoring the patient for clinical signs and symptoms of congestive heart failure (CHF), including shortness of breath and edema;
optionally obtaining a MRI or cardiac biopsy to make a diagnosis of CHF; and
continuing to administer entrectinib to the patient in the absence of adverse reaction in the patient or until cancer progression; or
if the patient experiences Grade 2 or 3 severity of CHF, withholding administration of entrectinib, instituting appropriate medical management, reassessing LVEF and severity of CHF, and resuming administration of entrectinib at a reduced dose upon patient recovery to less than or equal to Grade 1 severity of CHF or permanently discontinuing administration of entrectinib if the patient experiences Grade 4 severity of CHF.

In another aspect, provided herein are methods of managing adverse reactions of administration of entrectinib in a patient, the methods comprising:
prior to administration of entrectinib to the patient,
   advising the patient of a risk of one or more central nervous system (CNS) adverse reactions from the administration of entrectinib; and
   advising the patient not to drive or operate hazardous machinery if the patient experiences one or more CNS adverse reactions from the administration of entrectinib;
initiating administration of entrectinib to the patient with an initial dose;
monitoring the patient for one or more CNS adverse reactions; and
continuing to administer entrectinib to the patient in the absence of adverse reaction in the patient or until cancer progression; or
if the patient experiences Grade 2 severity of CNS adverse reaction, withholding administration of entrectinib and resuming administration of entrectinib at the initial dose or at a reduced dose upon patient recovery to less than or equal to Grade 1 severity of CNS adverse reaction; or
if the patient experiences Grade 3 severity of CNS adverse reaction, withholding administration of entrectinib and resuming administration of entrectinib at the reduced dose upon patient recovery to less than or equal to Grade 1 severity of CNS adverse reaction; or
if the patient experiences Grade 4 severity of CNS adverse reaction, permanently discontinuing administration of entrectinib.

In another aspect, provided herein are methods of managing adverse reactions of administration of entrectinib in a patient, the methods comprising:
initiating administration of entrectinib to the patient (e.g., at an initial dose);
monitoring the patient for one or more signs or symptoms of skeletal fracture;
if the patient exhibits one or more signs or symptoms of skeletal fracture, promptly evaluating the patient and instituting appropriate medical management; and
continuing to administer entrectinib to the patient in the absence of additional adverse reaction in the patient or until cancer progression.

In another aspect, provided herein are methods of managing adverse reactions of administration of entrectinib in a patient, the methods comprising:
initiating administration of entrectinib to the patient with an initial dose;
monitoring the patient with liver tests, including ALT and AST, every 2 weeks during the first month of administration, then monthly thereafter, and as clinically indicated; and
continuing to administer entrectinib to the patient in the absence of adverse reaction in the patient or until cancer progression; or
if the patient experiences Grade 3 severity of hepatotoxicity, withholding administration of entrectinib and resuming administration of entrectinib at the initial dose upon patient recovery within 4 weeks to less than or equal to Grade 1 severity of hepatotoxicity, or resuming administration of entrectinib at a reduced dose for recurrent Grade 3 severity of hepatotoxicity that resolve within 4 weeks; or permanently discontinuing administration of entrectinib if Grade 3 severity of hepatotoxicity does not resolve within 4 weeks; or
if the patient experiences Grade 4 severity of hepatotoxicity, withholding administration of entrectinib and resuming administration of entrectinib at the reduced dose upon patient recovery within 4 weeks to less than or equal to Grade 1 severity of hepatotoxicity, or permanently discontinuing administration of entrectinib if Grade 4 severity of hepatotoxicity does not resolve within 4 weeks or recurs; or
if the patient exhibits ALT or AST greater than 3 times the upper limit of normal (ULN) with concurrent total bilirubin greater than 1.5 times ULN in the absence of cholestasis or hemolysis, permanently discontinuing administration of entrectinib.

In another aspect, provided herein are methods of managing adverse reactions of administration of entrectinib in a patient, the methods comprising:
assessing serum uric acid levels in the patient prior to initiating administration of entrectinib;
initiating administration of entrectinib to the patient with an initial dose;
reassessing serum uric acid levels periodically during administration of entrectinib and monitoring the patient for signs and symptoms of hyperuricemia; and
continuing to administer entrectinib to the patient in the absence of adverse reaction in the patient or until cancer progression; or
if the patient is symptomatic of hyperuricemia or experiences Grade 4 hyperuricemia, initiating treatment with urate-lowering medications as clinically indicated, withholding entrectinib and resuming administration of entrectinib at the initial dose or at a reduced dose upon improvement of signs or symptoms of hyperuricemia.

In another aspect, provided herein are methods of managing adverse reactions of administration of entrectinib in a patient, the methods comprising:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc greater than 500 ms, withholding entrectinib until QTc interval recovers to baseline and resuming administration of entrectinib at the initial dose or at a reduced dose; or
if the patient exhibits Torsade de pointes, polymorphic ventricular tachycardia, or signs or symptoms of serious arrhythmia, permanently discontinuing administration of entrectinib.

In another aspect, provided herein are methods of managing adverse reactions of administration of entrectinib in a patient, the methods comprising:
initiating administration of entrectinib to the patient with an initial dose;
monitoring the patient for a new vision disorder; and
continuing to administer entrectinib to the patient in the absence of adverse reaction in the patient or until cancer progression; or
if the patient experiences Grade 2 or higher severity of vision disorder, withholding entrectinib until improvement or stabilization of the vision disorder, and resuming administration of entrectinib at the initial dose or at a reduced dose.

In another aspect, provided herein are methods of managing adverse reactions of administration of entrectinib in a patient, the methods comprising:
initiating administration of entrectinib to the patient with an initial dose;
monitoring the patient for anemia or neutropenia; and
continuing to administer entrectinib to the patient in the absence of adverse reaction in the patient or until cancer progression; or
if the patient experiences Grade 3 or Grade 4 severity of anemia or neutropenia, withholding entrectinib and resuming administration of entrectinib at the initial dose or at a reduced dose upon patient recovery to Grade 2 or less severity of anemia or neutropenia.

In another aspect, provided herein are methods of managing adverse reactions of administration of entrectinib in a patient, the methods comprising:
prior to the administration of entrectinib to the patient,
   advising the patient of a potential for cognitive change from the administration of entrectinib; and
   advising the patient not to drive or operate hazardous machinery if the patient experiences one or more symptoms of cognitive change from the administration of entrectinib;
initiating administration of entrectinib to the patient with an initial dose;
monitoring the patient for a cognitive disorder; and
continuing to administer entrectinib to the patient in the absence of adverse reaction in the patient or until cancer progression; or
if the patient experiences an event of at least Grade 2 severity of cognitive disorder, withholding administration of entrectinib until patient recovery to less than or equal to Grade 1 severity of cognitive disorder and resuming administration of entrectinib at a reduced dose; or
if the patient experiences a recurrence of the event of at least Grade 2 severity of cognitive disorder, administering entrectinib at a further reduced dose; or
if the patient experiences prolonged, severe, or intolerable events of cognitive disorder, permanently discontinuing administration of entrectinib.

In another aspect, provided herein are methods of managing adverse reactions of administration of entrectinib in a patient, the methods comprising:
initiating administration of entrectinib to the patient with an initial dose;
monitoring the patient for transaminase elevation; and
continuing to administer entrectinib to the patient in the absence of adverse reaction in the patient or until cancer progression; or
if the patient exhibits Grade 3 severity of transaminase elevation, withholding administration of entrectinib until patient recovery to less than or equal to Grade 1 severity of transaminase elevation; and resuming administration of entrectinib to the patient without change in dose upon patient recovery within 4 weeks to less than or equal to Grade 1 severity of transaminase elevation, or resuming administration of entrectinib at a reduced dose for recurrent Grade 3 severity of hepatotoxicity that resolve within 4 weeks, or permanently discontinuing administration of entrectinib to the patient if Grade 3 severity of transaminase elevation does not resolve within 4 weeks; or
if the patient exhibits Grade 4 severity of transaminase elevation, withholding administration of entrectinib until patient recovery to less than or equal to Grade 1 severity of transaminase elevation; and resuming administration of entrectinib to the patient without change in dose upon patient recovery within 4 weeks to less than or equal to Grade 1 severity of transaminase elevation, or permanently discontinuing administration of entrectinib to the patient if Grade 4 severity of transaminase elevation does not resolve within 4 weeks or recurs; or
if the patient exhibits ALT or AST elevation greater than 3 times the upper limit of normal (ULN) with total bilirubin elevation being greater than 2 times ULN in the absence of cholestasis or hemolysis, permanently discontinuing administration of entrectinib to the patient.

In another aspect, provided herein are methods of managing adverse reactions of administration of entrectinib in a patient, the methods comprising:
initiating administration of entrectinib to the patient with an initial dose;
monitoring the patient for congestive heart failure; and
continuing to administer entrectinib to the patient in the absence of adverse reaction in the patient or until cancer progression; or
if the patient exhibits Grade 2 or Grade 3 severity of congestive heart failure, withholding administration of entrectinib until patient recovery to less than or equal to Grade 1 severity of congestive heart failure; and resuming administration of entrectinib at a reduced dose; or
if the patient exhibits Grade 4 severity of congestive heart failure, withholding administration of entrectinib until patient recovery to less than or equal to Grade 1 severity of congestive heart failure; and resuming administration of entrectinib at a reduced dose or permanently discontinuing administration of entrectinib to the patient.

In another aspect, provided herein are methods of managing adverse reactions of administration of entrectinib in a patient, the methods comprising:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc of 481 to 500 ms, withholding entrectinib until QTc interval recovers to baseline and resuming administration of entrectinib at the initial dose; or
if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or
if the patient exhibits Torsade de pointes, polymorphic ventricular tachycardia, or signs or symptoms of serious arrhythmia, permanently discontinuing administration of entrectinib.

In another aspect, provided herein are methods of managing adverse reactions of administration of entrectinib in a patient, the methods comprising:
prior to the administration of entrectinib to the patient,
   advising the patient of a potential for embryo-fetal toxicity;
initiating administration of entrectinib to the patient (e.g., at an initial dose); and
continuing to administer entrectinib to the patient in the absence of adverse reaction in the patient or until cancer progression; or
if the patient becomes pregnant, permanently discontinuing administration of entrectinib.

In another aspect, provided herein are methods of managing adverse reactions of administration of entrectinib in a patient, the methods comprising:
initiating administration of entrectinib to the patient with an initial dose;
monitoring the patient for adverse reaction; and
continuing to administer entrectinib to the patient in the absence of adverse reaction in the patient or until cancer progression; or
if the patient experiences syncope, withholding administration of entrectinib until patient recovery to baseline, and resuming administration of entrectinib at a reduced dose; or
if the patient experiences a recurrence of syncope, discontinuing administration of entrectinib.

In another aspect, provided herein are methods of managing adverse reactions of administration of entrectinib in a patient, the methods comprising:
initiating administration of entrectinib to the patient with an initial dose;
monitoring the patient for adverse reaction; and
continuing to administer entrectinib to the patient in the absence of adverse reaction in the patient or until cancer progression; or
if the patient experiences Grade 3 or Grade 4 severity of adverse reaction, withholding entrectinib and resuming administration of entrectinib at the initial dose or at a reduced dose upon patient recovery to Grade 1 or less severity of adverse reaction or permanently discontinuing administration of entrectinib if adverse reaction does not resolve within 4 weeks or Grade 4 severity adverse reactions recur.

In some embodiments, the initial dose is 600 mg, 500 mg, or 400 mg. In some embodiments, the reduced dose is 400 mg, 300 mg, or 200 mg. In some embodiments, the initial dose is 600 mg, and the reduced dose is 400 mg or 200 mg. In some embodiments, the initial dose is 600 mg, and the reduced dose is 400 mg.

In some embodiments, the cognitive change or the cognitive disorder comprises one or more of confusion, mental status changes, memory impairment, and hallucinations.

In some embodiments, the initial dose is 600 mg, orally administered once daily, and the reduced dose is 400 mg, orally administered once daily. In some embodiments, the initial dose is 600 mg, orally administered once daily, and the reduced dose is 200 mg, orally administered once daily. In some embodiments, the initial dose is 100 mg, orally administered once daily, and the reduced dose is 100 mg, orally administered once per day for 5 days each week (e.g., on Monday, Wednesday, Friday, Saturday, and Sunday). In one variation, the reduced dose is 100 mg, orally administered once per day for 3 days each week (e.g., on Monday, Thursday and Saturday). In some embodiments, the initial dose is 200 mg, orally administered once daily, and the reduced dose is 200 mg, orally administered once per day for 5 days each week (e.g., on Monday, Wednesday, Friday, Saturday, and Sunday). In one variation, the initial dose is 200 mg, orally administered once daily, and the reduced dose is 100 mg, orally administered once per day for 5 days each week (e.g., on Monday, Wednesday, Friday, Saturday, and Sunday). In some embodiments, the initial dose is 300 mg, orally administered once daily, and the reduced dose is 200 mg, orally administered once daily. In one variation, the initial dose is 300 mg, orally administered once daily, and the reduced dose is 100 mg, orally administered once daily. In some embodiments, the initial dose is 400 mg, orally administered once daily, and the reduced dose is 300 mg, orally administered once daily. In one variation, the initial dose is 400 mg, orally administered once daily, and the reduced dose is 200 mg, orally administered once per day for 5 days each week (e.g., on Monday, Wednesday, Friday, Saturday, and Sunday). In some embodiments, the initial dose is 500 mg, orally administered once daily, and the reduced dose is 400 mg, orally administered once daily. In some embodiments, the initial dose is 500 mg, orally administered once daily, and the reduced dose is 200 mg, orally administered once daily. In some of these embodiments, the patient is an adult patient. In some of these embodiments, the patient is a pediatric patient. In some embodiments, the patient is a pediatric patients 12 years of age and older. In some embodiments, for a pediatric patient of 12 years or older having a body surface area of ≥1.50 m², the initial dose is 600 mg, orally administered once daily. In some embodiments, for a pediatric patient of 12 years or older having a body surface area of 1.11-1.50 m², the initial dose is 500 mg, orally administered once daily. In some embodiments, for a pediatric patient of 12 years or older having a body surface area of 0.91-1.10 m², the initial dose is 400 mg, orally administered once daily. In some embodiments, the patient is a pediatric patient having a body surface area of ≥1.51 m² and the initial doase is 600 mg, orally administered once daily. In some embodiments, the patient is a pediatric patient having a body surface area of 1.11-1.50 m² and the initial dose is 400 mg, orally administered once daily. In some embodiments, the patient is a pediatric patient having a body surface area of 0.81-1.10 m² and the initial dose is 300 mg, orally administered once daily. In some embodiments, the patient is a pediatric patient having a body surface area of 0.51-0.80 m² and the initial dose is 200 mg, orally administered once daily. In some embodiments, the patient is a pediatric patient having a body surface area of 0.43-0.50 m² and the initial dose is 100 mg, orally administered once daily.

In some embodiments, entrectinib is administered once daily. In some embodiments, entrectinib is administered twice daily. In some embodiments, the patient has at least one genetic alteration in at least one target gene selected from ROS1, NTRK1, NTRK2, and NTRK3. In some embodiments, the patient has a genetic alteration in ROS1. In some embodiments, the patient has a genetic alteration in NTRK1, NTRK2, NTRK3, or any combination thereof.

In some embodiments, the cancer is selected from sarcoma, non-small cell lung cancer, mammary analogue secretory carcinoma (MASC), breast cancer, thyroid cancer, colorectal cancer, neuroendocrine cancers, pancreatic cancer, gynecological cancers, and cholangiocarcinoma. In some embodiments, the cancer comprises one or more NTRK gene fusion-positive solid tumors. In some embodiments, the cancer is ROS-1 positive non-small cell lung cancer.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. For example, the term “a cell” includes one or more cells, including mixtures thereof. “A and/or B” is used herein to include all of the following alternatives: “A,” “B,” “A or B,” and “A and B.”

Recitation of ranges of values herein are merely intended to serve as a shorthand method of referring individually to each separate value falling within the range, including the endpoints unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “entrectinib” refers to “N-[5-(3,5-difluorobenzyl)-1H-indazol-3-yl]-4-(4-methyl-piperazin-1-yl)-2-(tetrahydro-pyran-4-ylamino)-benzamide” which is a compound having Chemical Abstracts Service Registry No. 1108743-60-7 and having the chemical structure:

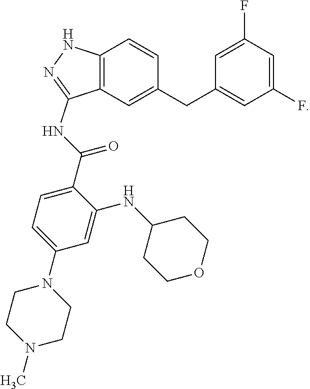 [SMILES: CN1CCN(c2ccc(C(=O)Nc3n[nH]c4ccc(Cc5cc(F)cc(F)c5)cc34)c(NC3CCOCC3)c2)CC1]

Hereinafter, all references to entrectinib herein include references to pharmaceutically acceptable salts, solvates, complexes, polymorphic forms, tautomers, and isotopically labeled versions thereof. Also included within the scope provided herein are pharmaceutical compositions comprising pharmaceutically acceptable salts, solvates, complexes, polymorphic forms, tautomers, and isotopically labeled versions of entrectinib.

As used herein, the terms “administration” and “administering” mean the delivery of a bioactive composition or formulation to a subject by an administration route including, but not limited to, oral, intravenous, intra-arterial, intramuscular, intraperitoneal, subcutaneous, intramuscular, topically, or combinations thereof. In some embodiments, the administration to a subject is oral.

As used herein, the term “ALK” means anaplastic lymphoma kinase receptor or CD246 (cluster of differentiation 246), which is an enzyme that in humans is encoded by the ALK gene and also has the UniProt identified ALK_HUMAN.

As used herein, the term “antibody” means an immunoglobulin that specifically binds to, and is thereby defined as complementary with, a particular spatial and polar organization of another molecule. The antibody can be monoclonal or polyclonal and can be prepared by techniques that are well known in the art, such as immunization of a host and collection of sera (polyclonal), or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular target is maintained.

As used herein, the term “AUC” means the area under the curve of a plot of the concentration of a compound in the plasma of a subject versus time.

As used herein, the term “biological sample,” means a sample obtained from an organism that may be used in a diagnostic or monitoring assay. The sample may be of a healthy tissue, diseased tissue or tissue suspected of being diseased tissue. The sample may be a biopsy taken, for example, during a surgical procedure. The sample may be collected via means of fine needle aspiration, scraping or washing a cavity to collects cells or tissue therefrom. The sample may be of a tumor such as, for example, solid and hematopoietic tumors as well as of neighboring healthy tissue. The sample may be a smear of subject cells or a tissue section. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses clinical samples, and also includes cells in cell culture, cell supernatants, cell lysates, cell extracts, cell homogenates, and subcellular components including synthesized proteins, serum, plasma, bodily and other biological fluids, and tissue samples. The biological sample can contain compounds that are not naturally intermixed with the cell or tissue in nature such as preservatives, anticoagulants, buffers, fixatives, nutrients, antibiotics or the like. In some embodiments, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample.

As used herein, the term “biomarker” means one or more compounds whose level of nucleic acid or protein product has a quantitatively differential concentration or level with respect to an aspect of a biological state of a subject. The term “biomarker” may be used herein interchangeably with the term “marker.” The level of the biomarker can be measured at both the nucleic acid level as well as the polypeptide level. At the nucleic acid level, a nucleic acid gene or a transcript which is transcribed from any part of the subject's chromosomal and extrachromosomal genome, including for example the mitochondrial genome, may be measured. Preferably an RNA transcript, more preferably an RNA transcript includes a primary transcript, a spliced transcript, an alternatively spliced transcript, or an mRNA of the biomarker is measured. At the polypeptide level, a pre-propeptide, a propeptide, a mature peptide or a secreted peptide of the biomarker may be measured. A biomarker can be used either solely or in conjunction with one or more other identified biomarkers so as to allow correlation to the biological state of interest as defined herein. Specific examples of biomarkers covered by the present disclosure include those associated with ALK, ROS1, TrkA, TrkB, and TrkC.

As used herein, the term “Cₘₐₓ” means the peak concentration that a compound achieves in the plasma of a subject after the compound, or a pharmaceutical composition comprising the compound, has been administrated to the subject. In some embodiments, the compound, or a pharmaceutical composition comprising the compound, is administered orally to a subject to achieve a particular Cₘₐₓ.

As used herein, the terms “cancer” or “tumor” may be used interchangeably. These terms mean the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers. In certain embodiments, the cancer is a solid tumor, a soft tissue tumor, or a metastatic lesion. The terms also refer to solid tumors named for the type of cells that form them, cancer of blood, bone marrow, or the lymphatic system. Examples of solid tumors include, but are not limited to, sarcomas and carcinomas. Examples of cancers of the blood include, but are not limited to, leukemias, lymphomas and myeloma. The terms include, but are not limited to, a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one. As used herein “cancer” refers to any malignant and/or invasive growth or tumor caused by abnormal cell growth.

As used herein, the term “chemotherapeutic agent”, means a chemical substance, such as a cytotoxic or cytostatic agent, that is used to treat a condition, particularly cancer.

As used herein, the terms “combination” and “in combination with” mean the administration of entrectinib together with at least one additional pharmaceutical or medicinal agent (e.g., an anti-cancer agent), either sequentially or simultaneously. It includes dosing simultaneously, or within minutes or hours of each other, or on the same day, or on alternating days, or dosing the pharmaceutical compositions provided herein on a daily basis, or multiple days per week, or weekly basis, for example, while administering another compound such as a chemotherapeutic agent on the same day or alternating days or weeks or on a periodic basis during a time simultaneous therewith or concurrent therewith, or at least a part of the time during which the pharmaceutical compositions disclosed herein is dosed. For example, a pharmaceutical composition as provided herein that comprises entrectinib could be dosed every day or several days a week while the chemotherapeutic agent is dosed on alternating days or alternating weeks or other periods of time, such as every 1, 2, 3, 4, 5, 6, 7, 8, 9, 10, 11, 12, 13, 14 or more days.

As used herein, the term “contact” when used in reference to specificity or specific binding means two molecules are close enough so that short range non-covalent chemical interactions, such as Van der Waal forces, hydrogen bonding, hydrophobic interactions, and the like, dominate the interaction of the molecule.

As used herein, the term “cell line” means to one or more generations of cells which are derived from a clonal cell. The term “clone,” or “clonal cell,” means a single cell which is expanded to produce an isolated population of phenotypically similar cells (i.e. a “clonal cell population”).

As used herein, the term “immunohistochemistry,” means the process of localizing antigens (e.g. proteins) in biological samples, cells and/or cells of a tissue section exploiting the principle of antibodies binding specifically to antigens. Immunohistochemical staining is widely used in the diagnosis of abnormal cells such as those found in cancerous tumors. Specific molecular markers are characteristic of particular cellular events, such as cell proliferation or cell death. Visualizing an antibody-antigen interaction can be accomplished in a number of ways. In the most common instance, an antibody is conjugated to an enzyme, such as peroxidase, that can catalyze a color-producing reaction. Alternatively, the antibody can also be tagged to a fluorophore thus employing the principles of immunofluorescence. Immunohistochemistry can also be used to evaluate tumor content in the sample on which qPCR is carried out in order to account for the fact that qPCR result will be influenced by the amount of tumor tissue present.

As used herein, the term “impaired hepatic function” and “hepatic dysfunction” refer to abnormal liver activity. In some embodiments, the Child-Pugh scoring system is used for assessment of hepatic dysfunction. The Child-Pugh scoring system is described in Table 1.

TABLE 1
Child-Pugh scoring system for assessment of hepatic dysfunction

| | Score | | |
|---|---|---|---|
| | 1 | 2 | 3 |
| Encephalopathy grade | 0 | 1 or 2 | 3 or 4 |
| Ascites | Absent | Slight | Moderate |
| Serum bilirubin | <2 | 2 to 3 | >3 |
| (mg/dL) | | | |
| Serum albumin | >3.5 | 2.8 to 3.5 | <2.8 |
| (g/dL) | | | |
| Prothrombin time | <4 | 4 to 6 | >6 |
| (seconds prolonged) | | | |

Individuals are assigned a score for each parameter, and the sum of these scores is used as the basis for classification of clinical severity:
5 to 6 points - mild hepatic impairment (Child-Pugh A)
7 to 9 points - moderate (Child-Pugh B)
>9 points - severe (Child-Pugh C).

In some embodiments, the National Cancer Institute (NCI) organ dysfunction working group (NCI-ODWG) system is used for assessment of hepatic dysfunction. The NCI-ODWG system is described in Table 2.

TABLE 2
NCI-ODWG definitions of hepatic impairment

| | Mild | Moderate | Severe |
|---|---|---|---|
| Total | B1: ≤ULN | >1.5-3 × ULN | >3 × ULN |
| bilirubin | B2: >1-1.5 × ULN | | |
| AST | B1: >ULN | Any | Any |
| | B2: Any | | |

AST, aspartate aminotransferase;
ULN, upper limit of normal.

The ULN for various indicators of liver function depends on the assay used, the patient population, and each laboratory's normal range of values for the specified biomarker, but can readily be determined by the skilled practitioner. Exemplary values for normal ranges for a healthy adult population are as follows: ALT: 8-20 U/L; AST: 8-20 U/L; bilirubin: 0.2-1.0 mg/dL or 3.4-17.1 μmol/L. (See Cecil Textbook of Medicine, pp. 2317-2341, W.B. Saunders & Co. (1985)). In some embodiments, the ULN for total bilirubin is 1.0 mg/dL, 1.1 mg/dL, or 1.2 mg/dL, or more. In some embodiments, the ULN for total bilirubin is 1.2 mg/dL or 20.5 μmol/L. In some embodiments, the ULN for ALT is 56 units/L. In some embodiments, the ULN for ALT is 40 units/L. In some embodiments, the ULN for ALT is 35 units/L to 40 units/L. In some embodiments, the ULN for AST is 40 units/L. In some embodiments, the ULN for AST is 35 units/L to 40 units/L.

As used herein, the term “transaminase” refers to alanine transaminase (ALT), aspartate transaminase (AST), or a combination thereof.

As used herein, the terms “monoclonal antibody,” “mAb” and “MAB” mean an antibody that is an immunoglobulin produced by a single clone of lymphocytes which recognizes only a single epitope on an antigen. For example, a monoclonal antibody useful for the methods provided herein displays a single binding specificity and affinity for a particular epitope of one or more tyrosine kinases.

As used herein, the term “multiplexed assay” means an assay in which multiple assay reactions, e.g. simultaneous assays of multiple target biomarkers, are carried out in a single reaction chamber and/or and analyzed in a single separation and detection format.

As used herein, the term “multiplex identification” means the simultaneous identification of one or more target biomarkers in a single mixture. For example, a two-plex assay means the simultaneous identification, in a single reaction mixture, of two different target biomarkers.

As used herein, the term “one or more molecular alterations” means any variation in the genetic or protein sequence in one or more cells of a subject as compared to the corresponding wild-type genes or proteins. One or more molecular alterations include, but are not limited to, genetic mutations, gene amplifications, splice variants, deletions, insertions/deletions, gene rearrangements, single-nucleotide variations (SNVs), insertions, and aberrant RNA/protein expression.

It is understood by those having ordinary skill in the art that references made to “pharmaceutical compositions provided herein,” and the like, mean those pharmaceutical compositions that are described as embodiments herein. By way of example only, a method of treating a subject having cancer comprising administering to said subject a pharmaceutical composition as provided herein, means a method of treating a subject having cancer comprising administering to said subject any of the compositions described herein that comprise entrectinib.

As used herein, the term “polyclonal antibody” means a composition of different antibody molecules which is capable of binding to or reacting with several different specific antigenic determinants on the same or on different antigens. The variability in antigen specificity of a polyclonal antibody is located in the variable regions of the subject antibodies constituting the polyclonal antibody, in particular in the complementarity determining regions (CDRs). Preferably, the polyclonal antibody is prepared by immunization of an animal with the target tyrosine kinases or portions thereof. Alternatively, the polyclonal antibody may be prepared by mixing multiple monoclonal antibodies having desired specificity to a target tyrosine kinase.

As used herein, “ROS1” means the ROS1 receptor tyrosine-protein kinase having the UniProt designation ROS1_HUMAN.

As used herein, the term “selectively binds” means the situation in which one member of a specific intra- or inter-species binding pair will not show any significant binding to molecules other than its specific intra- or inter-species binding partner (e.g., an affinity of about 100-fold less), which means that only minimal cross-reactivity occurs.

As used herein in reference to the binding of two molecules or one or more compounds and a complex of molecules, the term “specific” means the specific recognition of one for the other and the formation of a stable complex, as compared to substantially less recognition of other molecules and the lack of formation of stable complexes with such other molecules. Preferably, “specific,” in reference to binding, means that to the extent that one or more compounds forms complexes with other molecules or complexes, it forms at least fifty percent of the complexes with the molecule or complex for which it has specificity. Generally, the molecules or complexes have areas on their surfaces or in cavities giving rise to specific recognition between the two binding moieties. Exemplary of specific binding are antibody-antigen interactions, enzyme-substrate interactions, polynucleotide hybridizations and/or formation of duplexes, cellular receptor-ligand interactions, and so forth.

As used herein, the term “subject” means a mammal, including, but not limited to, a human, a dog or a cat. In some embodiments, the subject is a human. In some embodiments, the subject is a dog. In some embodiments, the subject is a cat.

As used herein, the term “Tₘₐₓ” means the time when the peak concentration of a compound in the plasma of a subject is reached after administration of the compound, or a pharmaceutical composition comprising the compound, to the subject.

As used herein, the term “therapeutically effective amount” means that amount of the compound or compounds, or pharmaceutically acceptable salts thereof, being administered to a subject which will relieve to some extent one or more of the symptoms of the disorder being treated. In reference to the treatment of a cancer, a therapeutically effective amount means that amount which has the effect of (1) reducing the size of a cancer tumor, (2) inhibiting (that is, slowing to some extent, preferably stopping) cancer tumor metastasis, (3) inhibiting to some extent (that is, slowing to some extent, preferably stopping) cancer tumor growth, and/or, (4) relieving to some extent (or, preferably, eliminating) one or more symptoms associated with the cancer.

As used herein, the terms “tropomyosin receptor kinase,” “Trks” and “Trk” mean the family of tropomyosin receptor kinases (Trks) that are activated by peptide hormones of the neurotrophin family and include, but are not limited to, TrkA, TrkB, and TrkC. As used herein, the term “TrkA” means wild-type tropomyosin receptor kinase A having the UniProt identifier NTRK1_HUMAN. As used herein, the term “TrkB” means wild-type tropomyosin receptor kinase B having the UniProt identifier NTRK2_HUMAN. As used herein, the term “TrkC” means wild-type tropomyosin receptor kinase C having the UniProt identifier NTRK3_HUMAN. TrkA, TrkB and TrkC are also referred to by those having ordinary skill in the art as Trk1, Trk2 and Trk3, respectively. A reference to TrkA is a reference to Trk1. A reference to TrkB is a reference to Trk2. A reference to TrkC is a reference to Trk3.

Entrectinib and its preparation have been disclosed in U.S. Pat. No. 8,299,057, the contents of which are hereby incorporated by reference in their entirety. Entrectinib is a potent inhibitor of tyrosine kinases, NTRK1/2/3-transforming tyrosine kinase proteins (TrkA, TrkB, TrkC), proto-oncogene tyrosine-protein kinase 1 (ROS1), and anaplastic lymphoma kinase (ALK). In various in vitro studies, entrectinib inhibited proliferation of the CRC cell line KM12, which depends upon TrkA kinase activity for proliferation and survival. It was also potent in inhibiting cell proliferation of ALK-dependent Anaplastic Large Cell Lymphoma cell lines.

In early clinical studies in humans, entrectinib has been shown to have antitumor effects in patients having various forms of cancer having at least one molecular alteration in one or more of ALK, ROS1, TrkA, TrkB and TrkC.

In vitro studies indicate that the clearance of entrectinib is predominantly through hepatic metabolism, and that entrectinib is primarily metabolized by cytochrome P450 3A4 (CYP3A4) (˜76%) with minor contributions from several other cytochrome P450 enzymes and UDP-glucuroyltransferase 1A4 (UGT1A4). The active metabolite M5 (formed by CYP3A4) is the only major active circulating metabolite identified. M5 has similar pharmacological potency to entrectinib in vitro and circulating M5 exposures at steady-state in patients were 40% of the corresponding entrectinib exposure. Furthermore co-administration of a strong CYP3A4 inhibitor (itraconazole), increased total exposure of entrectinib by approximately 5-fold while co-administration of a strong inducer (rifampin) decreased total exposure of entrectinib by approximately 77%. Thus the results indicate that entrectinib is a sensitive substrate of CYP3A4 and hence impairment of liver function has the potential to alter exposure of entrectinib. As such, it is an object of the present disclosure to provide methods of treating cancer in patients with impaired hepatic function utilizing entrectinib, or a pharmaceutically acceptable salt thereof, or pharmaceutical compositions containing the same.

Some embodiments provide methods of inhibiting ALK, ROS1, TrkA, TrkB, or TrkC activity, or a combination thereof, in a subject having impaired hepatic function, comprising administering to said subject a pharmaceutical composition as provided herein that comprises an effective amount of entrectinib.

In some embodiments are provided methods for the treatment of abnormal cell growth in a mammal comprising administering to a mammal a therapeutically effective amount of entrectinib or one or more pharmaceutical compositions provided herein, wherein the mammal has impaired hepatic function. In some embodiments, the abnormal cell growth is mediated by at least one molecular alteration in one or more of ALK, ROS1, TrkA, TrkB and TrkC in a mammal having impaired hepatic function. In some embodiments, the abnormal cell growth is mediated by at least one molecular alteration in ALK in a mammal having impaired hepatic function. In some embodiments, the abnormal cell growth is mediated by at least one molecular alteration in ROS1 in a mammal having impaired hepatic function. In some embodiments, the abnormal cell growth is mediated by at least one molecular alteration in TrkA in a mammal having impaired hepatic function. In some embodiments, the abnormal cell growth is mediated by at least one molecular alteration in TrkB in a mammal having impaired hepatic function. In some embodiments, the abnormal cell growth is mediated by at least one molecular alteration in TrkC in a mammal having impaired hepatic function. In some such embodiments, the molecular alteration is the EML4-ALK fusion protein. In some embodiments, the molecular alteration is the EML4-ALK fusion protein having at least one mutation. In some embodiments, the mutation is L1196M. In some embodiments, the mutation is C1156Y.

In some embodiments are provided pharmaceutical compositions provided herein for use as a medicament for treatment of abnormal cell growth in a mammal having impaired hepatic function. In some embodiments, the abnormal cell growth is cancer. In some embodiments, the medicament is for use in the treatment of abnormal cell growth mediated by one or more of ALK, ROS1, TrkA, TrkB and TrkC in a mammal having impaired hepatic function. In some embodiments, the medicament is for use in the treatment of abnormal cell growth mediated by at least one molecular alteration in one or more of ALK, ROS1, TrkA, TrkB and TrkC in a mammal having impaired hepatic function. In some embodiments, the medicament is for use in the treatment of abnormal cell growth mediated by at least one molecular alteration in ALK in a mammal having impaired hepatic function. In some embodiments, the medicament is for use in the treatment of abnormal cell growth mediated by at least one molecular alteration in ROS1 in a mammal having impaired hepatic function. In some embodiments, the medicament is for use in the treatment of abnormal cell growth mediated by at least one molecular alteration in TrkA in a mammal having impaired hepatic function. In some embodiments, the medicament is for use in the treatment of abnormal cell growth mediated by at least one molecular alteration in TrkB in a mammal having impaired hepatic function. In some embodiments, the medicament is for use in the treatment of abnormal cell growth mediated by at least one molecular alteration in TrkC in a mammal having impaired hepatic function. In some such embodiments, the molecular alteration is the EML4-ALK fusion protein. In some embodiments, the molecular alteration is the EML4-ALK fusion protein having at least one mutation. In some embodiments, the mutation is L1196M. In some embodiments, the mutation is C1156Y.

In some embodiments are provided methods for the treatment of abnormal cell growth in a mammal comprising administering to a mammal an amount of one or more pharmaceutical compositions provided herein, in combination with an amount of an anti-tumor agent, which amounts are together effective in treating said abnormal cell growth, wherein the mammal has impaired hepatic function. In some embodiments, the anti-tumor agent is selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

In some embodiments are provided uses of one or more pharmaceutical compositions described herein for the treatment of abnormal cell growth in a mammal having impaired hepatic function.

In yet another aspect, disclosed herein are uses of one or more pharmaceutical compositions described herein for the preparation of a medicament for the treatment of abnormal cell growth in a mammal having impaired hepatic function.

In frequent embodiments of the methods and uses described herein, the abnormal cell growth is cancer. In some embodiments, the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

In some embodiments, the cancer is selected from the group consisting of non-small cell lung cancer (NSCLC), squamous cell carcinoma, hormone-refractory prostate cancer, papillary renal cell carcinoma, colorectal adenocarcinoma, neuroblastomas, anaplastic large cell lymphoma (ALCL) and gastric cancer. In some embodiments, the cancer is one or more solid tumor(s).

In some embodiments are provided methods for treating diseases caused by and/or associated with deregulated protein kinase activity, particularly PLK family, protein kinase C in different isoforms, Met, PAK-4, PAK-5, ZC-1, STLK-2, DDR-2, Aurora 1, Aurora 2, Bub-1, Chk1, Chk2, HER2, raf1, MEK1, MAPK, EGF-R, PDGF-R, FGF-R, FLT3, JAK2, IGF-R, ALK, PI3K, weel kinase, Src, Abl, Akt, MAPK, ILK, MK-2, IKK-2, Cdc7, Nek, Cdk/cyclin kinase family, more particularly Aurora 2, IGF-1R and ALK activity, and ROS1 activity, and further more particularly ALK activity and/or ROS1 activity, which comprises administering to a mammal in need thereof an effective amount of entrectinib or one or more pharmaceutical compositions provided herein, wherein the mammal has impaired hepatic function.

In some embodiments are provided methods to treat a disease caused by and/or associated with dysregulated protein kinase activity selected from the group consisting of cancer and cell proliferative disorders, in a subject having impaired hepatic function.

The pharmaceutical compositions provided herein are useful for the treatment of cancers comprising but not limited to cancers of the: circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Karposi's sarcoma, moles dysplastic nevi, lipoma, angioma, dermatofibroma, and keloids; adrenal glands: neuroblastoma; and other tissues comprising connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites.

In some embodiments, the cancer is selected from lung cancer (NSCLC and SCLC), cancer of the head or neck, ovarian cancer, colon cancer, rectal cancer, prostate cancer, cancer of the anal region, stomach cancer, breast cancer, cancer of the kidney or ureter, renal cell carcinoma, carcinoma of the renal pelvis, neoplasms of the central nervous system (CNS), primary CNS lymphoma, non-Hodgkins's lymphoma, spinal axis tumors, or a combination of one or more of the foregoing cancers.

In some embodiments, the cancer is selected from non-small cell lung cancer, papillary thyroid cancer, neuroblastoma, pancreatic cancer and colorectal cancer. In some embodiments, the cancer is non-small cell lung cancer. In some embodiments, the cancer is papillary thyroid cancer. In some embodiments, the cancer is neuroblastoma. In some embodiments, the cancer is pancreatic cancer. In some embodiments, the cancer is colorectal cancer.

In some embodiments, the cancer is ROS-1 positive NSCLC. In some embodiments, the cancer is non-small cell lung cancer (NSCLC) that has spread to other parts of the body and is caused by an abnormal ROS1 gene. In some embodiments, the cancer is metastatic non-small cell lung cancer (NSCLC) whose tumors are ROS1-positive. In some embodiments, the cancer is ROS-1 positive, locally advanced or metastatic NSCLC.

In some embodiments, the cancer is NTRK gene fusion-positive solid tumors. In some embodiments, the cancer is solid tumors that are caused by certain abnormal NTRK genes and have spread or if surgery to remove their cancer is likely to cause severe complications, and there is no satisfactory alternative treatment option or the cancer grew or spread on other treatment. In some embodiments, the cancer is solid tumors that have a neurotrophic tyrosine receptor kinase (NTRK) gene fusion without a known acquired resistance mutation, are metastatic or where surgical resection is likely to result in severe morbidity, and have progressed following treatment or have no satisfactory alternative therapy. In some embodiments, the cancer is NTRK fusion-positive locally advanced or metastatic solid tumor(s).

In some embodiments, the pharmaceutical compositions provided herein are useful for the treatment of cancers, comprising Spitz melanoma, perineural invasion, pulmonary large cell neuroendocrine carcinoma, uterine carcinoma, juvenile breast cancer, nasopharyngeal carcinoma, adenoid cystic cancer, meduallary thyroid cancer, salivary cancer, congenital infantile fibrosarcoma, mesoblastic nephroma, esophageal cancer (squamous), diffuse large B-cell lymphoma, papillary thyroid cancer, and mammary analogue secretory carcinoma.

In some embodiments are provided methods to treat specific types of cancer comprising carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, angiosarcoma, glioblastoma, cholangiocarcinoma, inflammatory myofibroblastic tumor, epitheloid hem angi oendotheli om a, astrocytoma, meningioma, angiosarcoma, epitheloid hem angi otheli oma, keratocanthomas, thyroid follicular cancer, Kaposi's sarcoma, and pancreatic cancer, in a subject having impaired hepatic function.

In some embodiments are provided methods to treat specific types of cancer such as, but not restricted to, breast cancer, lung cancer, colorectal cancer, prostate cancer, ovarian cancer, endometrial cancer, gastric cancer, clear cell renal cell carcinoma, invasive ductal carcinoma (breast), uveal melanoma, multiple myeloma, rhabdomyosarcoma, Ewing's sarcoma, Kaposi's sarcoma, pancreatic cancer, neuroblastoma, and medulloblastoma, in a subject having impaired hepatic function.

In some embodiments are provided methods to treat ALK+ anaplastic large cell lymphomas (ALCL) and possibly other indications in which the ALK activity might play a role, like neuroblastoma, rhabdomyosarcoma, glioblastoma, inflammatory myofibroblastic tumor, and some kind of melanomas, breast carcinomas, Ewings sarcomas, retinoblastomas and non-small cell lung carcinomas (NSCLC), in a subject having impaired hepatic function.

In some embodiments are provided methods to treat, reduce the symptoms of, ameliorate the symptoms of, delay the onset of, or otherwise pharmaceutically address pancreatic cancer and possibly other indications in which a defect in the modulation of ALK, ROS1, TrkA, TrkB, or TrkC activity, or a combination thereof, or upregulation, misregulation or deletion thereof might play a role by administering entrectinib or one or more pharmaceutical compositions provided herein to a subject having impaired hepatic function. In some embodiments are provided methods to treat, reduce the symptoms of, ameliorate the symptoms of, delay the onset of, or otherwise pharmaceutically address pancreatic cancer and possibly other indications in which a defect in the modulation of ROS1, TrkA, TrkB, or TrkC activity, or a combination thereof, activity, or upregulation, misregulation or deletion thereof might play a role by administering entrectinib or one or more pharmaceutical compositions provided herein to a subject having impaired hepatic function.

In some embodiments are provided methods to treat, reduce the symptoms of, ameliorate the symptoms of, delay the onset of, or otherwise pharmaceutically address pancreatic cancer and possibly other indications in which a defect in the modulation of ROS1 activity, or upregulation, misregulation or deletion thereof might play a role by administering entrectinib or one or more pharmaceutical compositions provided herein to a subject having impaired hepatic function.

In some embodiments are provided methods to treat, reduce the symptoms of, ameliorate the symptoms of, delay the onset of, or otherwise pharmaceutically address pancreatic cancer associated with a ROS1 down-regulation defect, for example a null mutation such as a ROS1 deletion by identifying a ROS1 down-regulation defect, for example a null mutation such as a ROS1 deletion in a cancer or precancerous pancreatic cell in a subject having impaired hepatic function, and administering to the subject entrectinib or one or more pharmaceutical compositions provided herein.

In some embodiments are provided methods to treat, reduce the symptoms of, ameliorate the symptoms of, delay the onset of, or otherwise pharmaceutically address pancreatic cancer in a subject having impaired hepatic function, which cancer is associated with a ALK, ROS1, TrkA, TrkB, or TrkC down-regulation defect, for example a null mutation such as a ALK, ROS1, TrkA, TrkB, or TrkC deletion by identifying a ALK, ROS1, TrkA, TrkB, or TrkC down-regulation defect, for example a null mutation such as a ALK, ROS1, TrkA, TrkB, or TrkC deletion in a cancer or precancerous pancreatic cell in the subject, and administering to the subject entrectinib or one or more pharmaceutical compositions provided herein.

In some embodiments are provided methods to treat, reduce the symptoms of, ameliorate the symptoms of, delay the onset of, or otherwise pharmaceutically address a condition in a subject having impaired hepatic function selected from non-small cell lung cancer, papillary thyroid cancer, neuroblastoma, pancreatic cancer and colorectal cancer and possibly other indications in which a defect in the modulation of ALK, ROS1, TrkA, TrkB, or TrkC activity, or a combination thereof, or upregulation, misregulation or deletion thereof might play a role by administering to the subject entrectinib or one or more of the pharmaceutical compositions provided herein.

In some embodiments are provided methods to treat, reduce the symptoms of, ameliorate the symptoms of, delay the onset of, or otherwise pharmaceutically address a condition in a subject having impaired hepatic function selected from non-small cell lung cancer, papillary thyroid cancer, neuroblastoma, pancreatic cancer and colorectal cancer and possibly other indications in which a defect in the modulation of ROS1, TrkA, TrkB, or TrkC activity, or a combination thereof, activity, or upregulation, misregulation or deletion thereof might play a role by administering to the subject entrectinib or one or more of the pharmaceutical compositions provided herein.

In some embodiments identifying a ROS1 modulation defect such as an upregulation defect or a down-regulation defect, for example a null mutation such as a ROS1 deletion or a ROS1 chimeric locus encoding a constitutively active ROS1 kinase in a cancer or precancerous pancreatic cell in an subject having impaired hepatic function comprises assaying for ROS1 activity in a cell extract from a pancreatic cancerous or precancerous cell population. In some embodiments identifying a ROS1 modulation defect such as an upregulation defect or a down-regulation defect, for example a null mutation such as a ROS1 deletion or a ROS1 chimeric locus encoding a constitutively active ROS1 kinase in a cancer or precancerous pancreatic cell in a subject having impaired hepatic function comprises assaying for ROS1 transcript accumulation in an RNA population from a pancreatic cancerous or precancerous cell population. In some embodiments identifying a ROS1 modulation defect such as an upregulation defect or a down-regulation defect, for example a null mutation such as a ROS1 deletion or a ROS1 chimeric locus encoding a constitutively active ROS1 kinase in a cancer or precancerous pancreatic cell in a subject having impaired hepatic function comprises determining the nucleic acid sequence such as the genomic deoxyribonucleic acid sequence in a cell or cells or a cell population comprising a cell or cells from a pancreatic cancerous or precancerous cell population.

In some embodiments are provided methods to treat, reduce the symptoms of, ameliorate the symptoms of, delay the onset of, or otherwise pharmaceutically address a condition in a subject having impaired hepatic function selected from non-small cell lung cancer, papillary thyroid cancer, neuroblastoma, pancreatic cancer and colorectal cancer associated with a ALK, ROS1, TrkA, TrkB, or TrkC down-regulation defect, for example a null mutation such as a ALK, ROS1, TrkA, TrkB, or TrkC deletion by identifying a ALK, ROS1, TrkA, TrkB, or TrkC down-regulation defect, for example a null mutation such as a ALK, ROS1, TrkA, TrkB, or TrkC deletion in a cancer or precancerous cell in the subject, and administering to the subject entrectinib or one or more of the pharmaceutical compositions provided herein.

In some embodiments are provided methods to treat, reduce the symptoms of, ameliorate the symptoms of, delay the onset of, or otherwise pharmaceutically address a condition in a subject having impaired hepatic function selected from non-small cell lung cancer, papillary thyroid cancer, neuroblastoma, pancreatic cancer and colorectal cancer associated with a ROS1 down-regulation defect, for example a null mutation such as a ROS1 deletion by identifying a ROS1 down-regulation defect, for example a null mutation such as a ROS1 deletion in a cancer or precancerous cell in the subject, and administering to the subject entrectinib or one or more pharmaceutical compositions provided herein.

In some embodiments are provided methods to treat cell proliferative disorders such as, but not restricted to, benign prostate hyperplasia, familial adenomatosis polyposis, neuro-fibromatosis, psoriasis, atherosclerosis and conditions involving vascular smooth muscle proliferation or neointimal formation such as restenosis following angioplasty or surgery, pulmonary fibrosis, arthritis, glomerulonephritis, retinopathies comprising diabetic and neonatal retinopathies and age related macular degeneration, graft vessel disease, such as can occur following vessel or organ transplantation, acromegaly and disorders secondary to acromegaly as well as other hypertrophic conditions in which IGF/IGF-1R signaling is implicated, such as fibrotic lung disease, pathologies related to chronic or acute oxidative stress or hyperoxia induced tissue damage, and metabolic disorders in which elevated IGF levels or IGF-1R activity are implicated, such as obesity, in a subject having impaired hepatic function.

In some embodiments are provided methods of affecting tumor angiogenesis and metastasis inhibition.

Some embodiments provide the use of one or more pharmaceutical compositions as provided herein in the manufacture of a medicament with antitumor activity.

In some embodiments, the methods provided herein further comprise subjecting the mammal in need thereof to a radiation therapy or chemotherapy regimen in combination with at least one cytostatic or cytotoxic agent. In some embodiments, the methods provided herein further comprise inhibiting the activity ALK protein which comprises contacting the said protein with an effective amount of entrectinib or one or more pharmaceutical compositions provided herein.

Some embodiments provide methods of treating cancer in a subject in need thereof, the method comprising inhibiting ALK, ROS1, TrkA, TrkB, or TrkC activity, or a combination thereof, in said subject, by administering to said subject an effective amount of entrectinib or one or more pharmaceutical compositions provided herein, and wherein said subject has impaired hepatic function.

Some embodiments provide methods of treating tumors in a subject having impaired hepatic function, said methods comprising administering to the subject an effective amount of entrectinib or one or more pharmaceutical compositions provided herein. Some embodiments provide methods of treating tumors in a subject having impaired hepatic function, said methods comprising administering to the subject an effective amount of entrectinib or a pharmaceutical composition as provided herein that comprises an effective amount of entrectinib.

Some embodiments provide methods wherein the tumors are caused by the presence of non-small cell lung cancer, papillary thyroid cancer, neuroblastoma, pancreatic cancer or colorectal cancer in the subject having impaired hepatic function. Some embodiments provide methods wherein one or more of the cells comprising the tumors in the subject test positive for the presence of a gene that expresses at least one of ALK, ROS1, TrkA, TrkB, or TrkC kinase or one or more of the cells comprising the tumors in said subject demonstrates at least one of ALK, ROS1, TrkA, TrkB, or TrkC kinase activity.

Some embodiments provide methods wherein one or more of the cells comprising the tumors in the subject test positive for at least one gene rearrangement comprising the gene, or a fragment thereof, that expresses at least one of ALK, ROS1, TrkA, TrkB, or TrkC kinase. Some embodiments provide such methods wherein the cells test positive for at least one of ROS1, TrkA, TrkB, or TrkC kinases. Some embodiments provide methods wherein the cells test positive for ROS1 kinase. Some embodiments provide methods wherein the cells test positive for at least one of TrkA, TrkB and TrkC kinase. Some embodiments provide methods wherein the cells test positive for TrkA kinase. Some embodiments provide methods wherein the cells test positive for TrkB kinase. Some embodiments provide such methods wherein the cells test positive for TrkC kinase.

Some embodiments provide methods of treating cancer in a subject having impaired hepatic function, the method comprising: (1) testing one or more cells comprising the tumors in the subject for the presence of at least one of ALK, ROS1, TrkA, TrkB, or TrkC kinase; and (2) administering to the subject an effective amount of entrectinib or one or more pharmaceutical compositions provided herein.

Some embodiments provide methods of treating cancer in a subject having impaired hepatic function, the method comprising: (1) testing one or more cells comprising the tumors in the subject for the presence of at least one molecular alteration in ALK, ROS1, TrkA, TrkB, or TrkC kinase; and (2) administering to the subject an effective amount of entrectinib or one or more pharmaceutical compositions provided herein.

Some embodiments provide methods of treating cancer in a subject having impaired hepatic function, wherein one or more cancerous cells in said subject express at least one of ROS1, TrkA, TrkB, or TrkC kinase, the method comprising administering to the subject an effective amount of entrectinib or one or more pharmaceutical compositions provided herein.

Some embodiments provide a method for treating a subject having cancer, wherein tumors from said subject are ROS1, TrkA, TrkB, or TrkC positive, a combination thereof, the method comprising administering to the subject an effective amount of entrectinib or one or more pharmaceutical compositions provided herein, and wherein the subject has impaired hepatic function.

Some embodiments provide a method of treating a cancer subject having impaired hepatic function, comprising (a) acquiring knowledge of the presence of at least one genetic alteration in at least one target gene in the cancer subject, wherein the at least one target gene is selected from ALK1, BDNF, NGF, NGFR, NTF3, NTF4, ROS1, SORT1, NTRK1, NTRK2, and NTRK3; (b) selecting one or more pharmaceutical compositions provided herein as a treatment for the cancer subject, based on the recognition that said pharmaceutical composition is effective in treating cancer subjects having said at least one genetic alteration in said at least one target gene; and (c) administering a therapeutically effective amount of entrectinib or one or more of said pharmaceutical compositions to said cancer subject.

Some embodiments provide a method of treating a cancer subject having impaired hepatic function, comprising administering to said cancer subject a therapeutically effective amount of one or more pharmaceutical compositions provided herein, wherein prior to said administration of said one or more pharmaceutical compositions said cancer subject is known to possess at least one genetic alteration in at least one target gene selected from ALK1, BDNF, NGF, NGFR, NTF3, NTF4, ROS1, SORT1, NTRK1, NTRK2, and NTRK3.

Some embodiments provide a method of treating a cancer subject having impaired hepatic function, wherein prior to said treatment said subject is known to possess at least one genetic alteration in at least one target gene, comprising administering to said cancer subject a therapeutically effective amount of entrectinib or one or more pharmaceutical compositions provided herein and wherein said at least one target gene is selected from ALK1, BDNF, NGF, NGFR, NTF3, NTF4, ROS1, SORT1, NTRK1, NTRK2, and NTRK3.

In one aspect, provided herein are methods for treating cancer in a subject having impaired hepatic function, comprising (a) acquiring knowledge of the presence of one or more molecular alterations in a biological sample from the cancer subject, wherein the one or more molecular alterations is detected by an assay comprising one or more antibodies that bind to one or more of ALK, ROS1, TrkA, TrkB, and TrkC biomarkers; (b) selecting a chemotherapeutic agent as a treatment for the cancer subject wherein the assay detects the presence of one or more of molecular alterations, and wherein the selected chemotherapeutic agent is one or more of the pharmaceutical compositions provided herein, or a pharmaceutically acceptable salt thereof; and (c) administering a therapeutically effective amount of the one or more selected chemotherapeutic agents to the cancer subject.

In another aspect, provided herein are methods for selecting a cancer subject who is predicted to respond to the administration of a therapeutic regimen, comprising (a) acquiring knowledge of the presence of one or more molecular alterations in a biological sample from the cancer subject, wherein the one or more molecular alterations is detected by an assay comprising one or more antibodies that bind to one or more of ALK, ROS1, TrkA, TrkB, and TrkC biomarkers; and (b) selecting the subject as predicted to respond to the administration of a therapeutic regimen if the one or more molecular alterations is detected in one or more of the biomarkers, or selecting the subject as predicted to not respond to the administration of a therapeutic regimen if the one or more molecular alterations is not detected in the biomarkers, and wherein the subject has impaired hepatic function. In the methods according to this aspect of the disclosure, the therapeutic regiment includes administering to the selected subject a therapeutically effective amount of entrectinib or one or more of the pharmaceutical compositions provided herein.

In some embodiments identifying a ALK, ROS1, TrkA, TrkB, or TrkC modulation defect such as an upregulation defect or a down-regulation defect, for example a null mutation such as a ALK, ROS1, TrkA, TrkB, or TrkC deletion or a ALK, ROS1, TrkA, TrkB, or TrkC chimeric locus encoding a constitutively active ALK, ROS1, TrkA, TrkB, or TrkC kinase in a cancer or precancerous cell in a subject having impaired hepatic function comprises assaying for ALK, ROS1, TrkA, TrkB, or TrkC activity in a cell extract from a cancerous or precancerous cell population. In some embodiments identifying a ALK, ROS1, TrkA, TrkB, or TrkC modulation defect such as an upregulation defect or a down-regulation defect, for example a null mutation such as a ALK, ROS1, TrkA, TrkB, or TrkC deletion or a ALK, ROS1, TrkA, TrkB, or TrkC chimeric locus encoding a constitutively active ALK, ROS1, TrkA, TrkB, or TrkC kinase in a cancer or precancerous cell in a subject having impaired hepatic function comprises assaying for ALK, ROS1, TrkA, TrkB, or TrkC transcript accumulation in an RNA population from a cancerous or precancerous cell population. In some embodiments identifying a ALK, ROS1, TrkA, TrkB, or TrkC modulation defect such as an upregulation defect or a down-regulation defect, for example a null mutation such as a ALK, ROS1, TrkA, TrkB, or TrkC deletion or a ALK, ROS1, TrkA, TrkB, or TrkC chimeric locus encoding a constitutively active ALK, ROS1, TrkA, TrkB, or TrkC kinase in a cancer or precancerous cell in a subject having impaired hepatic function comprises determining the nucleic acid sequence such as the genomic deoxyribonucleic acid sequence in a cell or cells or a cell population comprising a cell or cells from a cancerous or precancerous cell populations.

In some embodiments identifying a ALK, ROS1, TrkA, TrkB, or TrkC modulation defect such as an upregulation defect or a down-regulation defect, for example a null mutation such as a ALK, ROS1, TrkA, TrkB, or TrkC deletion or a ALK, ROS1, TrkA, TrkB, or TrkC chimeric locus encoding a constitutively active ALK, ROS1, TrkA, TrkB, or TrkC kinase in a cancer or precancerous pancreatic cell in a subject having impaired hepatic function comprises assaying for ALK, ROS1, TrkA, TrkB, or TrkC activity in a cell extract from a pancreatic cancerous or precancerous cell population. In some embodiments identifying a ALK, ROS1, TrkA, TrkB, or TrkC modulation defect such as an upregulation defect or a down-regulation defect, for example a null mutation such as a ALK, ROS1, TrkA, TrkB, or TrkC deletion or a ALK, ROS1, TrkA, TrkB, or TrkC chimeric locus encoding a constitutively active ALK, ROS1, TrkA, TrkB, or TrkC kinase in a cancer or precancerous pancreatic cell in a subject having impaired hepatic function comprises assaying for ALK, ROS1, TrkA, TrkB, or TrkC transcript accumulation in an RNA population from a pancreatic cancerous or precancerous cell population. In some embodiments identifying a ALK, ROS1, TrkA, TrkB, or TrkC modulation defect such as an upregulation defect or a down-regulation defect, for example a null mutation such as a ALK, ROS1, TrkA, TrkB, or TrkC deletion or a ALK, ROS1, TrkA, TrkB, or TrkC chimeric locus encoding a constitutively active ALK, ROS1, TrkA, TrkB, or TrkC kinase in a cancer or precancerous pancreatic cell in a subject having impaired hepatic function comprises determining the nucleic acid sequence such as the genomic deoxyribonucleic acid sequence in a cell or cells or a cell population comprising a cell or cells from a pancreatic cancerous or precancerous cell population.

Some embodiments provide methods of treating cancer in a subject in need thereof, the method comprising inhibiting ALK, ROS1, TrkA, TrkB, or TrkC activity, or a combination thereof, in said subject, by administering to said subject a pharmaceutical composition as provided herein that comprises an effective amount of entrectinib and wherein the subject has impaired hepatic function.

Some embodiments provide methods of treating non-small cell lung cancer, papillary thyroid cancer, neuroblastoma, pancreatic cancer or colorectal cancer in a subject having impaired hepatic function, comprising administering to said subject a pharmaceutical composition as provided herein that comprises an effective amount of entrectinib.

Some embodiments provide methods wherein the tumors are caused by the presence of non-small cell lung cancer, papillary thyroid cancer, neuroblastoma, pancreatic cancer or colorectal cancer in the subject. Some embodiments provide methods wherein one or more of the cells comprising the tumors in the subject test positive for the presence of a gene that expresses at least one of ALK, ROS1, TrkA, TrkB, or TrkC kinase or one or more of the cells comprising the tumors in said subject demonstrates at least one of ALK, ROS1, TrkA, TrkB, or TrkC kinase activity.

Some embodiments provide methods wherein one or more of the cells comprising the tumors in the subject test positive for at least one gene rearrangement comprising the gene, or a fragment thereof, that expresses at least one of ALK, ROS1, TrkA, TrkB, or TrkC kinase. Some embodiments provide such methods wherein the cells test positive for at least one of ROS1, TrkA, TrkB, or TrkC kinases. Some embodiments provide methods wherein the cells test positive for ROS1 kinase. Some embodiments provide methods wherein the cells test positive for at least one of TrkA, TrkB and TrkC kinase. Some embodiments provide methods wherein the cells test positive for TrkA kinase. Some embodiments provide methods wherein the cells test positive for TrkB kinase. Some embodiments provide such methods wherein the cells test positive for TrkC kinase.

Some embodiments provide methods of treating cancer in a subject, the method comprising: (1) testing one or more cells comprising the tumors in the subject for the presence of at least one of ALK, ROS1, TrkA, TrkB, or TrkC kinase; and (2) administering to the subject a pharmaceutical composition as provided herein that comprises an effective amount of entrectinib if said one or more cells tests positive for at least one of ALK, ROS1, TrkA, TrkB, or TrkC kinase, and wherein the subject has impaired hepatic function.

Some embodiments provide methods of treating cancer in a subject, the method comprising: (1) testing one or more cells comprising the tumors in the subject for the presence of at least one of ROS1, TrkA, TrkB, or TrkC kinase; and (2) administering to the subject a pharmaceutical formulation as provided herein that comprises an effective amount of entrectinib if said one or more cells tests positive for at least one of ROS1, TrkA, TrkB, or TrkC kinase, and wherein the subject has impaired hepatic function.

Some embodiments provide methods of treating cancer in a subject, wherein one or more cancerous cells in said subject express at least one of ROS1, TrkA, TrkB, or TrkC kinase, and wherein said subject has impaired hepatic function, the method comprising administering to the subject a pharmaceutical composition as provided herein that comprises an effective amount of entrectinib.

Some embodiments provide a method for treating a subject having cancer, wherein tumors from said subject are ALK, ROS1, TrkA, TrkB, or TrkC positive, or a combination thereof, and wherein said subject has impaired hepatic function, the method comprising administering to the subject a pharmaceutical composition as provided herein that comprises an effective amount of entrectinib.

Some embodiments provide a method for treating a subject having ALK, ROS1, TrkA, TrkB, or TrkC positive cancer, or a combination thereof, wherein said subject has impaired hepatic function, the method comprising administering to the subject a pharmaceutical formulation as provided herein that comprises an effective amount of entrectinib.

Some embodiments provide a method of treating a cancer subject, comprising (a) acquiring knowledge of the presence of at least one genetic alteration in at least one target gene in the cancer subject, wherein the at least one target gene is selected from ALK1, BDNF, NGF, NGFR, NTF3, NTF4, ROS1, SORT1, NTRK1, NTRK2, and NTRK3; and (b) administering a pharmaceutical composition as provided herein to said cancer subject, said pharmaceutical composition comprising a therapeutically effective amount of entrectinib; and wherein said cancer subject has impaired hepatic function.

Some embodiments provide a method of treating a cancer subject, comprising administering to said cancer subject a pharmaceutical composition as provided herein that comprises a therapeutically effective amount of entrectinib, wherein prior to said administration of said pharmaceutical composition, said cancer subject is known to possess at least one genetic alteration in at least one target gene selected from ALK1, BDNF, NGF, NGFR, NTF3, NTF4, ROS1, SORT1, NTRK1, NTRK2, and NTRK3, and wherein said subject has impaired hepatic function.

Some embodiments provide a method of treating cancer in a subject, comprising administering to said cancer subject known to possess impaired hepatic function and known to possess at least one genetic alteration in at least one target gene selected from ALK1, BDNF, NGF, NGFR, NTF3, NTF4, ROS1, SORT1, NTRK1, NTRK2, and NTRK3 a pharmaceutical composition as provided herein that comprises a therapeutically effective amount of entrectinib.

Some embodiments provide a method of treating a cancer subject, wherein said cancer subject is known to possess impaired hepatic function and known to possess at least one genetic alteration in at least one target gene, comprising administering to said cancer subject a pharmaceutical compositions comprising a therapeutically effective amount of entrectinib, and wherein said target gene is selected from ALK1, BDNF, NGF, NGFR, NTF3, NTF4, ROS1, SORT1, NTRK1, NTRK2, and NTRK3.

Some embodiments provide a method of treating a cancer subject, wherein prior to said treatment said subject is known to possess impaired hepatic function and known to possess at least one genetic alteration in at least one target gene, comprising administering to said cancer subject a pharmaceutical composition as provided herein that comprises a therapeutically effective amount of entrectinib, and wherein said target gene is selected from ALK1, BDNF, NGF, NGFR, NTF3, NTF4, ROS1, SORT1, NTRK1, NTRK2, and NTRK3.

Some embodiments provide a method of treating a cancer subject, comprising administering to said cancer subject a pharmaceutical composition as provided herein that comprises a therapeutically effective amount of entrectinib, and wherein prior to said pharmaceutical composition being administered to said subject, said subject is known to possess impaired hepatic function and known to possess at least one genetic alteration in at least one target gene selected from ALK1, BDNF, NGF, NGFR, NTF3, NTF4, ROS1, SORT1, NTRK1, NTRK2, and NTRK3.

Some embodiments provide a method for treating a cancer subject, comprising (a) acquiring knowledge of the presence of at least one genetic alteration in at least one target gene selected from ALK1, BDNF, NGF, NGFR, NTF3, NTF4, ROS1, SORT1, NTRK1, NTRK2, and NTRK3; and (b) administering to said subject a pharmaceutical composition as provided herein that comprises a therapeutically effective amount of entrectinib, and wherein said subject has impaired hepatic function.

Some embodiments provide any of the methods described herein wherein the subject or subject is suffering from cancer and the cancer is selected from at least one of non-small cell lung cancer, papillary thyroid cancer, neuroblastoma, pancreatic cancer and colorectal cancer. Some embodiments provide any of the methods described herein wherein the subject or subject is suffering from non-small cell lung cancer. Some embodiments provide any of the methods described herein wherein the subject or subject is suffering from papillary thyroid cancer. Some embodiments provide any of the methods described herein wherein the subject or subject is suffering from neuroblastoma. Some embodiments provide any of the methods described herein wherein the subject or subject is suffering from pancreatic cancer. Some embodiments provide any of the methods described herein wherein the subject or subject is suffering from colorectal cancer.

In some embodiments, the methods described herein further comprise administering to the mammal an amount of an anti-cancer therapeutic agent or a palliative agent, which amounts are together effective in treating said abnormal cell growth. In some such embodiments, one or more anti-cancer therapeutic agent are selected from anti-tumor agents, anti-angiogenesis agents, signal transduction inhibitors and antiproliferative agents, which amounts are together effective in treating said abnormal cell growth.

In other embodiments, the uses described herein comprise the use of one or more pharmaceutical compositions provided herein in combination with one or more substances selected from anti-tumor agents, anti-angiogenesis agents, signal transduction inhibitors and antiproliferative agents.

In some embodiments, the pharmaceutical compositions described herein are adapted for use in combination with one or more substances selected from anti-tumor agents, anti-angiogenesis agents, signal transduction inhibitors and antiproliferative agents.

Each of the embodiments of the pharmaceutical compositions provided herein can be combined with one or more other embodiments of the pharmaceutical compositions described herein that is not inconsistent with the embodiment(s) with which it is combined.

When a combination therapy is used, the one or more additional agents may be administered sequentially or simultaneously with the pharmaceutical compositions provided herein. In some embodiments, the additional agent is administered to a mammal (e.g., a human) prior to administration of the pharmaceutical compositions provided herein. In some embodiments, the additional agent is administered to the mammal after administration of the pharmaceutical compositions provided herein. In some embodiments, the additional agent is administered to the mammal (e.g., a human) simultaneously with the administration of pharmaceutical compositions provided herein.

Some embodiments also relate to pharmaceutical compositions for the treatment of abnormal cell growth in a mammal, comprising a human, which comprises an amount of one or more pharmaceutical compositions provided herein comprising entrectinib in combination with one or more (preferably one to three) anti-cancer agents selected from the group consisting of anti-angiogenesis agents and signal transduction inhibitors and a pharmaceutically acceptable carrier, wherein the amounts of entrectinib and the combination anti-cancer agents when taken as a whole is therapeutically effective for treating said abnormal cell growth, and wherein the mammal has impaired hepatic function.

In some embodiments, the anti-cancer agent used in conjunction with the pharmaceutical compositions described herein is an anti-angiogenesis agent (e.g., an agent that stops tumors from developing new blood vessels). Examples of anti-angiogenesis agents include for example VEGF inhibitors, VEGFR inhibitors, TIE-2 inhibitors, PDGFR inhibitors, angiopoetin inhibitors, PKC-beta inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors. Preferred anti-angiogenesis agents include sunitinib (Sutent®), bevacizumab (Avastin®), axitinib (AG 13736), SU 14813 (Pfizer), and AG 13958 (Pfizer).

Additional anti-angiogenesis agents include vatalanib (CGP 79787), Sorafenib (Nexavar®), pegaptanib octasodium (Macugen®), vandetanib (Zactima®), PF-0337210 (Pfizer), SU 14843 (Pfizer), AZD 2171 (AstraZeneca), ranibizumab (Lucentis®), Neovastat® (AE 941), tetrathiomolybdata (Coprexa®), AMG 706 (Amgen), VEGF Trap (AVE 0005), CEP 7055 (Sanofi-Aventis), XL 880 (Exelixis), telatinib (BAY 57-9352), and CP-868,596 (Pfizer).

Other anti-angiogenesis agents include enzastaurin (LY 317615), midostaurin (CGP 41251), perifosine (KRX 0401), teprenone (Selbex®) and UCN 01 (Kyowa Hakko).

Other examples of anti-angiogenesis agents which can be used in conjunction with one or more pharmaceutical compositions described herein include celecoxib (Celebrex®), parecoxib (Dynastat®), deracoxib (SC 59046), lumiracoxib (Preige®), valdecoxib (Bextra®), rofecoxib (Vioxx®), iguratimod (Careram®), IP 751 (Invedus), SC-58125 (Pharmacia) and etoricoxib (Arcoxia®).

Other anti-angiogenesis agents include exisulind (Aptosyn®), salsalate (Amigesic®), diflunisal (Dolobid®), ibuprofen (Motrin®), ketoprofen (Orudis®) nabumetone (Relafen®), piroxicam (Feldene®), naproxen (Aleve®, Naprosyn®) diclofenac (Voltaren®), indomethacin (Indocin®), sulindac (Clinoril®), tolmetin (Tolectin®), etodolac (Lodine®), ketorolac (Toradol®), and oxaprozin (Daypro®).

Other anti-angiogenesis agents include ABT 510 (Abbott), apratastat (TMI 005), AZD 8955 (AstraZeneca), incyclinide (Metastat®), and PCK 3145 (Procyon).

Other anti-angiogenesis agents include acitretin (Neotigason®), plitidepsin (Aplidine®), cilengtide (EMD 121974), combretastatin A4 (CA4P), fenretinide (4 HPR), halofuginone (Tempostatin®), Panzem® (2-methoxyestradiol), PF-03446962 (Pfizer), rebimastat (BMS 275291), catumaxomab (Removab®), lenalidomide (Revlimid®) squalamine (EVIZON®), thalidomide (Thalomid®), Ukrain® (NSC 631570), Vitaxin® (MEDI 522), and zoledronic acid (Zometa®).

In some embodiments, the anti-cancer agent is a so called signal transduction inhibitor (e.g., inhibiting the means by which regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, ALK inhibitors, ROS1 inhibitors, TrkA inhibitors, TrkB inhibitors, TrkC inhibitors, farnesyl protein transferase inhibitors, EGF inhibitor, ErbB-1 (EGFR), ErbB-2, pan erb, IGF1R inhibitors, MEK, c-Kit inhibitors, FLT-3 inhibitors, K-Ras inhibitors, PI3 kinase inhibitors, JAK inhibitors, STAT inhibitors, Raf kinase inhibitors, Akt inhibitors, mTOR inhibitor, P70S6 kinase inhibitors, inhibitors of the WNT pathway and so called multi-targeted kinase inhibitors.

Preferred signal transduction inhibitors include gefitinib (Iressa®), cetuximab (Erbitux®), erlotinib (Tarceva®), trastuzumab (Herceptin®), sunitinib (Sutent®) imatinib (Gleevec®), and PD325901 (Pfizer).

Additional examples of signal transduction inhibitors which may be used in conjunction with one or more pharmaceutical compositions provided herein include BMS 214662 (Bristol-Myers Squibb), lonafarnib (Sarasar®), pelitrexol (AG 2037), matuzumab (EMD 7200), nimotuzumab (TheraCIM h-R3®), panitumumab (Vectibix®), Vandetanib (Zactima®), pazopanib (SB 786034), ALT 110 (Alteris Therapeutics), BMW 2992 (Boehringer Ingelheim), and Cervene® (TP 38).

Other examples of signal transduction inhibitor include PF-2341066 (Pfizer), PF-299804 (Pfizer), canertinib (CI 1033), pertuzumab (Omnitarg®), Lapatinib (Tycerb®), pelitinib (EKB 569), miltefosine (Miltefosin®), BMS 599626 (Bristol-Myers Squibb), Lapuleucel-T (Neuvenge®), NeuVax® (E75 cancer vaccine), Osidem® (IDM 1), mubritinib (TAK-165), CP-724,714 (Pfizer), panitumumab (Vectibix®), lapatinib (Tycerb®), PF-299804 (Pfizer), pelitinib (EKB 569), and pertuzumab (Omnitarg®).

Other examples of signal transduction inhibitors include ARRY 142886 (Array Biopharm), everolimus (Certican®), zotarolimus (Endeavor®), temsirolimus (Toriselg), AP 23573 (ARIAD), and VX 680 (Vertex).

Additionally, other signal transduction inhibitors include XL 647 (Exelixis), sorafenib (Nexavar®), LE-AON (Georgetown University), and GI-4000 (GlobeImmune).

Other signal transduction inhibitors include ABT 751 (Abbott), alvocidib (flavopiridol), BMS 387032 (Bristol Myers), EM 1421 (Erimos), indisulam (E 7070), seliciclib (CYC 200), BIO 112 (One Bio), BMS 387032 (Bristol-Myers Squibb), PD 0332991 (Pfizer), AG 024322 (Pfizer), LOXO-101 (Loxo Oncology), crizotinib, and ceritinib.

In some embodiments, the pharmaceutical compositions provided herein are used together with classical antineoplastic agents. Classical antineoplastic agents include but are not limited to hormonal modulators such as hormonal, anti-hormonal, androgen agonist, androgen antagonist and anti-estrogen therapeutic agents, histone deacetylase (HDAC) inhibitors, gene silencing agents or gene activating agents, ribonucleases, proteosomics, Topoisomerase I inhibitors, Camptothecin derivatives, Topoisomerase II inhibitors, alkylating agents, antimetabolites, poly(ADP-ribose) polymerase-1 (PARP-1) inhibitor, microtubulin inhibitors, antibiotics, plant derived spindle inhibitors, platinum-coordinated compounds, gene therapeutic agents, antisense oligonucleotides, vascular targeting agents (VTAs), and statins.

Examples of classical antineoplastic agents used in combination therapy with one or more pharmaceutical compositions provided herein optionally with one or more other agents include, but are not limited to, glucocorticoids, such as dexamethasone, prednisone, prednisolone, methylprednisolone, hydrocortisone, and progestins such as medroxyprogesterone, megestrol acetate (Megace), mifepristone (RU-486), Selective Estrogen Receptor Modulators (SERMs; such as tamoxifen, raloxifene, lasofoxifene, afimoxifene, arzoxifene, bazedoxifene, fispemifene, ormeloxifene, ospemifene, tesmilifene, toremifene, trilostane and CHF 4227 (Cheisi)), Selective Estrogen-Receptor Downregulators (SERD's; such as fulvestrant), exemestane (Aromasin), anastrozole (Arimidex), atamestane, fadrozole, letrozole (Femara), gonadotropin-releasing hormone (GnRH; also commonly referred to as luteinizing hormone-releasing hormone [LHRH]) agonists such as buserelin (Suprefact), goserelin (Zoladex), leuprorelin (Lupron), and triptorelin (Trelstar), abarelix (Plenaxis), bicalutamide (Casodex), cyproterone, flutamide (Eulexin), megestrol, nilutamide (Nilandron), and osaterone, dutasteride, epristeride, finasteride, Serenoa repens, PHL 00801, abarelix, goserelin, leuprorelin, triptorelin, bicalutamide, tamoxifen, exemestane, anastrozole, fadrozole, formestane, letrozole, and combinations thereof.

Other examples of classical antineoplastic agents used in combination with pharmaceutical compositions provided herein include, but are not limited to, suberolanilide hydroxamic acid (SAHA, Merck Inc./Aton Pharmaceuticals), depsipeptide (FR901228 or FK228), G2M-777, MS-275, pivaloyloxymethyl butyrate and PXD-101; Onconase (ranpirnase), PS-341 (MLN-341), Velcade (bortezomib), 9-aminocamptothecin, belotecan, BN-80915 (Roche), camptothecin, diflomotecan, edotecarin, exatecan (Daiichi), gimatecan, 10-hydroxycamptothecin, irinotecan HC1 (Camptosar), lurtotecan, Orathecin (rubitecan, Supergen), SN-38, topotecan, camptothecin, 10-hydroxycamptothecin, 9-aminocamptothecin, irinotecan, SN-38, edotecarin, topotecan, aclarubicin, paclitaxel, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, Zinecard (dexrazoxane), nitrogen mustard N-oxide, cyclophosphamide, AMD-473, altretamine, AP-5280, apaziquone, brostallicin, bendamustine, busulfan, carboquone, carmustine, chlorambucil, dacarbazine, estramustine, fotemustine, glufosfamide, ifosfamide, KW-2170, lomustine, mafosfamide, mechlorethamine, melphalan, mitobronitol, mitolactol, mitomycin C, mitoxatrone, nimustine, ranimustine, temozolomide, thiotepa, and platinum-coordinated alkylating compounds such as cisplatin, Paraplatin (carboplatin), eptaplatin, lobaplatin, nedaplatin, Eloxatin (oxaliplatin, Sanofi), streptozocin, satrplatin, and combinations thereof.

In some embodiments, the pharmaceutical compositions provided herein are used together with dihydrofolate reductase inhibitors (such as methotrexate and NeuTrexin (trimetrexate)), purine antagonists (such as 6-mercaptopurine riboside, mercaptopurine, 6-thioguanine, cladribine, clofarabine (Clolar), fludarabine, nelarabine, and raltitrexed), pyrimidine antagonists (such as 5-fluorouracil (5-FU), Alimta (premetrexed disodium, LY231514, MTA), capecitabine (Xeloda®), cytosine arabinoside, Gemzar® (gemcitabine, Eli Lilly), Tegafur (UFT Orzel or Uforal and including TS-1 combination of tegafur, gimestat and otostat), doxifluridine, carmofur, cytarabine (including ocfosfate, phosphate stearate, sustained release and liposomal forms), enocitabine, 5-azacitidine (Vidaza), decitabine, and ethynylcytidine) and other antimetabolites such as eflornithine, hydroxyurea, leucovorin, nolatrexed (Thymitaq), triapine, trimetrexate, N-(5-[N-(3,4-dihydro-2-methyl-4-oxoquinazolin-6-ylmethyl)-N-methylamino]-2-thenoyl)-L-glutamic acid, AG-014699 (Pfizer Inc.), ABT-472 (Abbott Laboratories), INO-1001 (Inotek Pharmaceuticals), KU-0687 (KuDOS Pharmaceuticals) and GPI 18180 (Guilford Pharm Inc) and combinations thereof.

Other examples of classical antineoplastic cytotoxic agents used in combination therapy with one or more pharmaceutical compositions provided herein optionally with one or more other agents include, but are not limited to, Abraxane (Abraxis BioScience, Inc.), Batabulin (Amgen), EPO 906 (Novartis), Vinflunine (Bristol-Myers Squibb Company), actinomycin D, bleomycin, mitomycin C, neocarzinostatin (Zinostatin), vinblastine, vincristine, vindesine, vinorelbine (Navelbine), docetaxel (Taxotere), Ortataxel, paclitaxel (including Taxoprexin a DHA/paclitaxel conjugate), cisplatin, carboplatin, Nedaplatin, oxaliplatin (Eloxatin), Satraplatin, Camptosar, capecitabine (Xeloda), oxaliplatin (Eloxatin), Taxotere alitretinoin, Canfosfamide (Telcyta®), DMXAA (Antisoma), ibandronic acid, L-asparaginase, pegaspargase (Oncaspar®), Efaproxiral (Efaproxyn®—radiation therapy)), bexarotene (Targretin®), Tesmilifene (DPPE—enhances efficacy of cytotoxics)), Theratope® (Biomira), Tretinoin (Vesanoid®), tirapazamine (Trizaone®), motexafin gadolinium (Xcytrin®) Cotara® (mAb), and NBI-3001 (Protox Therapeutics), polyglutamate-paclitaxel (Xyotax®) and combinations thereof.

Further examples of classical antineoplastic agents used in combination therapy with one or more pharmaceutical compositions provided herein optionally with one or more other agents include, but are not limited to, as Advexin (ING 201), TNFerade (GeneVec, one or more compounds which express TNFalpha in response to radiotherapy), RB94 (Baylor College of Medicine), Genasense (Oblimersen, Genta), Combretastatin A4P (CA4P), Oxi-4503, AVE-8062, ZD-6126, TZT-1027, Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin, Niacin (Advicor, Kos Pharmaceuticals), Caduet, Lipitor, torcetrapib, and combinations thereof.

Some embodiments relate to a method for the treatment of breast cancer in a human in need of such treatment, wherein the human has impaired hepatic function. In some embodiments, the method includes, for example, administering to said human an amount of one or more pharmaceutical compositions provided herein in combination with one or more (preferably one to three) anti-cancer agents selected from the group consisting of trastuzumab, tamoxifen, docetaxel, paclitaxel, capecitabine, gemcitabine, vinorelbine, exemestane, letrozole and anastrozole.

Some embodiments provide a method of treating colorectal cancer in a mammal, such as a human, in need of such treatment, by administering an amount of one or more pharmaceutical compositions provided herein in combination with one or more (preferably one to three) anti-cancer agents, and wherein the mammal has impaired hepatic function. Examples of particular anti-cancer agents include those typically used in adjuvant chemotherapy, such as FOLFOX, a combination of 5-fluorouracil (5-FU) or capecitabine (Xeloda), leucovorin and oxaliplatin (Eloxatin). Further examples of particular anti-cancer agents include those typically used in chemotherapy for metastatic disease, such as FOLFOX or FOLFOX in combination with bevacizumab (Avastin); and FOLFIRI, a combination of 5-FU or capecitabine, leucovorin and irinotecan (Camptosar). Further examples include 17-DMAG, ABX-EFR, AMG-706, AMT-2003, ANX-510 (CoFactor), aplidine (plitidepsin, Aplidin), Aroplatin, axitinib (AG-13736), AZD-0530, AZD-2171, bacillus Calmette-Guerin (BCG), bevacizumab (Avastin), BIO-117, BIO-145, BMS-184476, BMS-275183, BMS-528664, bortezomib (Velcade), C-1311 (Symadex), cantuzumab mertansine, capecitabine (Xeloda), cetuximab (Erbitux), clofarabine (Clofarex), CMD-193, combretastatin, Cotara, CT-2106, CV-247, decitabine (Dacogen), E-7070, E-7820, edotecarin, EMD-273066, enzastaurin (LY-317615) epothilone B (EPO-906), erlotinib (Tarceva), flavopyridol, GCAN-101, gefitinib (Iressa), huA33, huC242-DM4, imatinib (Gleevec), indisulam, ING-1, irinotecan (CPT-11, Camptosar) ISIS 2503, ixabepilone, lapatinib (Tykerb), mapatumumab (HGS-ETR1), MBT-0206, MEDI-522 (Abregrin), Mitomycin, MK-0457 (VX-680), MLN-8054, NB-1011, NGR-TNF, NV-1020, oblimersen (Genasense, G3139), OncoVex, ONYX 015 (CI-1042), oxaliplatin (Eloxatin), panitumumab (ABX-EGF, Vectibix), pelitinib (EKB-569), pemetrexed (Alimta), PD-325901, PF-0337210, PF-2341066, RAD-001 (Everolimus), RAV-12, Resveratrol, Rexin-G, S-1 (TS-1), seliciclib, SN-38 liposome, Sodium stibogluconate (SSG), sorafenib (Nexavar), SU-14813, sunitinib (Sutent), temsirolimus (CCI 779), tetrathiomolybdate, thalomide, TLK-286 (Telcyta), topotecan (Hycamtin), trabectedin (Yondelis), vatalanib (PTK-787), vorinostat (SAHA, Zolinza), WX-UK1, and ZYC300, wherein the amounts of the active agent together with the amounts of the combination anticancer agents are effective in treating colorectal cancer.

Some embodiments provide methods for the treatment of renal cell carcinoma in a human in need of such treatment, comprising administering to said human an amount of pharmaceutical compositions provided herein in combination with one or more (preferably one to three) anti-cancer agents selected from the group consisting of capecitabine (Xeloda), interferon alpha, interleukin-2, bevacizumab (Avastin), gemcitabine (Gemzar), thalidomide, cetuximab (Erbitux), vatalanib (PTK-787), Sutent, AG-13736, SU-11248, Tarceva, Iressa, Lapatinib and Gleevec, wherein the amounts of the active agent together with the amounts of the combination anticancer agents is effective in treating renal cell carcinoma, and wherein the human has impaired hepatic function.

Some embodiments provide methods for the treatment of melanoma in a human in need of such treatment, comprising administering to said human an amount of pharmaceutical compositions provided herein, in combination with one or more (preferably one to three) anti-cancer agents selected from the group consisting of interferon alpha, interleukin-2, temozolomide (Temodar), docetaxel (Taxotere), paclitaxel, Dacarbazine (DTIC), carmustine (also known as BCNU), Cisplatin, vinblastine, tamoxifen, PD-325,901, Axitinib, bevacizumab (Avastin), thalidomide, sorafanib, vatalanib (PTK-787), Sutent, CpG-7909, AG-13736, Iressa, Lapatinib and Gleevec, wherein the amounts of the pharmaceutical compositions provided herein together with the amounts of the combination anticancer agents is effective in treating melanoma, wherein the human has impaired hepatic function.

Some embodiments provide methods for the treatment of lung cancer in a human in need of such treatment, comprising administering to said human an amount of one or more pharmaceutical compositions provided herein in combination with one or more (preferably one to three) anti-cancer agents selected from the group consisting of capecitabine (Xeloda), bevacizumab (Avastin), gemcitabine (Gemzar), docetaxel (Taxotere), paclitaxel, premetrexed disodium (Alimta), Tarceva, Iressa, Vinorelbine, Irinotecan, Etoposide, Vinblastine, and Paraplatin (carboplatin), wherein the amounts of the active agent together with the amounts of the combination anticancer agents is effective in treating lung cancer, wherein the human has impaired hepatic function.

Some embodiments provide a pharmaceutical composition comprising a therapeutically effective amount of entrectinib in combination with one or more chemotherapeutic agents or radiotherapy, such as radiotherapy as commonly administered to treat, ameliorate the symptoms of, or prevent or delay the onset of cancer. Such agents can include, but are not limited to, antihormonal agents such as antiestrogens, antiandrogens and aromatase inhibitors, topoisomerase I inhibitors, topoisomerase II inhibitors, agents that target microtubules, platin-based agents, alkylating agents, DNA damaging or intercalating agents, antineoplastic antimetabolites, other kinase inhibitors, other anti-angiogenic agents, inhibitors of kinesins, therapeutic monoclonal antibodies, inhibitors of mTOR, histone deacetylase inhibitors, farnesyl transferase inhibitors, and inhibitors of hypoxic response.

The presence of at least one genetic alteration in at least one target gene in a cancer subject, wherein the at least one target gene is selected from ALK1, BDNF, NGF, NGFR, NTF3, NTF4, ROS1, SORT1, NTRK1, NTRK2, and NTRK3 may be detected by use of an assay that includes one or more antibodies that bind to at least two, three, four, or all of ALK, ROS1, TrkA, TrkB and TrkC biomarkers. The one or more molecular alterations detected in the biological sample may involve at least two, at least three, or at least four of the biomarkers. The knowledge of the presence of the one or more molecular alterations in the biological sample may be acquired from an assay that includes contacting the biological sample with one or more antibodies or fragments thereof that are specific for the biomarkers. In some instances, the specific antibodies are monoclonal antibodies. In some instances, the specific antibodies include at least one of D5F3® (ALK rabbit mAb), D4D6® (ROS1 rabbit mAb), C17F1® (pan-Trk rabbit mAb), and combinations thereof. In some instances, the biological sample is contacted with one or more of the specific antibodies simultaneously. In some instances, the biological sample is sequentially contacted with the specific antibodies. In some instances, the one or more molecular alterations results in elevated expression of one or more of the ALK, ROS1, TrkA, TrkB, and TrkC biomarkers. In some instances, the knowledge of the one or more molecular alterations is acquired from an assay wherein determining whether the expression of one or more biomarker is elevated includes: (a) determining the expression level of the one or more biomarkers in the biological sample; and (b) comparing the determined expression level to a reference expression level.

As used herein, the term “reference level” refers to known expression level of the target biomarker(s) in a control person or subject. In some instances, the reference expression level is the expression level of the target biomarker(s) in a healthy person or subject. In some instances, the reference expression level is the expression level of the target biomarker(s) in a population of healthy control cells. In some instances, the reference expression level is the expression level of the target biomarker(s) in a control person or subject that has been previously determined to possess one or more molecular alterations. In some instances, the reference expression level is the expression level of the target biomarker(s) in a population of control cells that have been previously determined to possess one or more molecular alterations.

In some instances, the knowledge of the one or more molecular alterations is acquired from an antibody-based assay. The antibody-based assay can generally be any antibody-based assay, and can be, for example, ELISA, immunohistochemistry, western blotting, mass spectrometry, flow cytometry, protein-microarray, immunofluorescence, and a multiplex detection assay. In some instances, the antibody-based assay includes an immunohistochemistry analysis.

In some instances, identifying a ALK, ROS1, TrkA, TrkB, or TrkC modulation defect such as an upregulation defect or a down-regulation defect, for example a null mutation such as a ALK, ROS1, TrkA, TrkB, or TrkC deletion or a ALK, ROS1, TrkA, TrkB, or TrkC chimeric locus encoding a constitutively active ALK, ROS1, TrkA, TrkB, or TrkC kinase in a cancer or precancerous pancreatic cell in a subject having impaired hepatic function comprises assaying for ALK, ROS1, TrkA, TrkB, or TrkC activity in a cell extract from a pancreatic cancerous or precancerous cell population.

In some instances, identifying a ALK, ROS1, TrkA, TrkB, or TrkC modulation defect such as an upregulation defect or a down-regulation defect, for example a null mutation such as a ALK, ROS1, TrkA, TrkB, or TrkC deletion or a ALK, ROS1, TrkA, TrkB, or TrkC chimeric locus encoding a constitutively active ALK, ROS1, TrkA, TrkB, or TrkC kinase in a cancer or precancerous pancreatic cell in a subject having impaired hepatic function comprises assaying for ALK, ROS1, TrkA, TrkB, or TrkC transcript accumulation in an RNA population from a pancreatic cancerous or precancerous cell population.

In some instances, identifying a ALK, ROS1, TrkA, TrkB, or TrkC modulation defect such as an upregulation defect or a down-regulation defect, for example a null mutation such as a ALK, ROS1, TrkA, TrkB, or TrkC deletion or a ALK, ROS1, TrkA, TrkB, or TrkC chimeric locus encoding a constitutively active ALK, ROS1, TrkA, TrkB, or TrkC kinase in a cancer or precancerous pancreatic cell in a subject having impaired hepatic function comprises determining the nucleic acid sequence such as the genomic deoxyribonucleic acid sequence in a cell or cells or a cell population comprising a cell or cells from a pancreatic cancerous or precancerous cell population.

The term “microarray,” as used herein, means an ordered arrangement of array elements (for example, small samples of a biological sample from a subject such as tissue samples) mounted on a solid support capable of binding other molecule species or antibodies. The array elements are arranged so that there are preferably at least one or more different array elements.

The term “solid support,” as used herein, means the well-understood solid materials to which various components such as, for example, proteins and nucleic acids, are physically attached, thereby immobilizing the components. The term “solid support,” as used herein, means a non-liquid substance. A solid support can be, but is not limited to, a membrane, sheet, gel, glass, plastic or metal. Immobilized components may be associated with a solid support by covalent bonds and/or via non-covalent attractive forces such as hydrogen bond interactions, hydrophobic attractive forces and ionic forces, for example.

In some instances, the microarrays suitable for the methods provided herein have a density of at least 1, 2, 4, 6, 8, 10 spots/cm², preferably at least 10, 20, 30, 40, 50, 60, 70, 80, 90, 100, 110, 120, 130, 140, 150, 160, 170, 180, 190, 200, more preferably at least 210, 220, 230, 250, 275, 300, 325, 350, 375, 400, 425, 450, 475, 500, 550, 600, 650, 700, 750, 800, 850, 900, 950, 1000, 2000, 3000, 4000, 5000, 6000, 7000, 8000 or 9000 spots/cm².

In some instances, it is contemplated that the spots on the array may each represent a different species of biomarkers or that the multiple spots on the array may represent the same species of biomarkers. In some instances, the spots each represent an array element of differing identity or characteristics.

In some instances, implementations of the methods according to this and other aspects of the present disclosure further include acquiring knowledge of a genetic alteration in the cancer of the subject from a second analytical assay prior to the administering step. The second analytical assay can generally be any analytical assay known to those having ordinary skill in the art, and can be for example an antibody-based assay, a nucleotide-based assay, or an enzymatic activity assay. Non-limiting examples of suitable second analytical assays include capillary electrophoresis, nucleic acid sequencing, polypeptide sequencing, restriction digestion, nucleic acid amplification-based assays, nucleic acid hybridization assay, comparative genomic hybridization, real-time PCR, quantitative reverse transcription PCR (qRT-PCR), PCR-RFLP assay, HPLC, mass-spectrometric genotyping, fluorescent in-situ hybridization (FISH), next generation sequencing (NGS), and a kinase activity assay. Other examples of suitable second analytical assays include ELISA, immunohistochemistry, Western blotting, mass spectrometry, flow cytometry, protein-microarray, immunofluorescence, and multiplex detection assay.

In some instances, FISH analysis is used to identify the chromosomal rearrangement resulting in the one or more molecular alterations such as the fusion genes or gene products as described herein. For example, to perform FISH, at least a first probe tagged with a first detectable label can be designed to target a first gene of a fusion gene, such as in one or more exons of the gene and at least a second probe tagged with a second detectable label can be designed to target a second gene of the fusion gene, such as in one or more exons of the genes (for example, the exons containing the part of the protein that includes the tyrosine kinase domain). The at least one first probe and the at least one second probe will be closer together in a subject who carries the fusion compared to a subject who does not carry the fusion gene or gene product. In some instances, a variation of a FISH assay, for example, “break-apart FISH”, is used to evaluate a subject selected by a method provided herein. By this method, at least one probe targeting the fusion junction and at least one probe targeting a subject gene of the fusion, e.g., at one or more exons and or introns of the gene, are utilized. In normal cells, both probes will be observed (or a secondary color will be observed due to the close proximity of the two genes of the gene fusion), and only the single gene probe will be observed when the translocation occurs or the probes, having differing colors, will be separated such that one of ordinary skill in the art observing the probes can determine that a relevant gene fusion or deletion is present in the sample. Generally, FISH assays are performed using formalin-fixed, paraffin-embedded tissue sections that are placed on slides. The DNA from the tissue sample sections is denatured to single-stranded form and subsequently allowed to hybridize with the appropriate DNA probes that can be designed and prepared using methods and techniques known to those having ordinary skill in the art. Following hybridization, any unbound probe may be removed by a series of washes and the nuclei of the cells are counter-stained with DAPI (4′,6 diamidino-2-phenylindole), a DNA-specific stain that fluoresces blue. Hybridization of the probe or probes are viewed using a fluorescence microscope equipped with appropriate excitation and emission filters, allowing visualization of the fluorescent signals.

For example, a break-apart FISH assay may be used to detect multiple types of rearrangements involving the ALK gene locus. In the method, tumor cells from some subjects having non-small cell lung cancer (NSCLC), display an ALK-positive FISH pattern as detected using single interference filter sets comprising green (FITC), red (Texas red), and blue (4′,6-diamidino-2-phenylindole) as well as dual (red/green) and triple (blue, red, green) band-pass filters. A fusion of the ALK gene is visualized as split orange and green signals, single orange signals, or single orange and single green signals.

Relevant molecular alterations with respect to ROS1, TrkA, TrkB and TrkC in biological samples derived from cancer subjects using the same methods as described above, but by modifying the reagents, probes and other materials used in the assays in ways that are appropriate to the target molecular alteration and as can be readily determined by those having ordinary skill in the art.

Other variations of the FISH method known in the art are suitable for evaluating a subject selected in accordance with the methods provided herein.

In some instances are provided such methods, wherein the knowledge of the presence of the one or more molecular alterations is obtained from an assay performed simultaneously on a plurality of biological samples. In some instances, the plurality of biological samples may be assayed in a multitest platform.

As used herein, the term “multitest platform” is intended to encompass any suitable means to contain one or more reaction mixtures, suspensions, or detection reactions. As such, the outcomes of a number of screening events can be assembled onto one surface, resulting in a “multitest platform” having, or consisting of multiple elements or parts to do more than one experiment simultaneously. It is intended that the term “multitest platform” encompasses protein chips, microtiter plates, multi-well plates, microcards, test tubes, petri plates, trays, slides, and the like. In some instances, multiplexing can further include simultaneously conducting a plurality of screening events in each of a plurality of separate biological samples. For example, the number of biological samples analyzed can be based on the number of spots on a slide and the number of tests conducted in each spot (as described in greater detail in Example 2). In another example, the number of biological samples analyzed can be based on the number of wells in a multi-well plate and the number of tests conducted in each well. For example, 6-well, 12-well, 24-well, 48-well, 96-well, 384-well, 1536-well or 3456-well microtiter plates can be useful in the presently disclosed methods, although it will be appreciated by those in the art, not each microtiter well need contain a subject biological sample. Depending on the size of the microtiter plate and the number of the subject biological samples in each well, very high numbers of tests can be run simultaneously. Although multiplexing has been exemplified in Example 2 with respect to micro-slides, it will be understood that other formats can be used for multiplexing.

In some instances are provided such methods, wherein the plurality of biological samples includes at least 6, 12, 24, 48, 96, 200, 384, 400, 500, 1000, 1250, 1500, or 3000 samples.

In some instances are provided such methods, wherein the one or more molecular alterations is selected from a genetic mutation, a gene amplification, a gene rearrangement, a single-nucleotide variation (SNV), a deletion, an insertion, an InDel mutation, a single nucleotide point mutation (SNP), an epigenetic alteration, a splicing variant, an RNA/protein overexpression, and an aberrant RNA/protein expression. In some instances are provided such methods, wherein the genetic alteration includes an insertion of a heterologous nucleic acid sequence within a coding sequence of a biomarker gene. In some instances are provided such methods, wherein the insertion forms a chimeric nucleic acid sequence that encodes a fusion peptide.

In some instances are provided such methods, wherein the acquiring knowledge of the one or more molecular alterations further comprises determining a nucleic acid sequence and/or an amino acid sequence comprising the one or more molecular alterations. In some instances, the nucleic acid sequence comprising the one or more molecular alterations from a selected cancer subject tumor is sequenced. In some instances, the sequence is determined by a next generation sequencing method.

Provided herein, in another aspect, are methods of managing adverse reactions of the administration of entrectinib in a patient. In some embodiments, the method comprising administering (e.g., orally) to a patient in need thereof (e.g., a person having a solid tumor with at least one genetic alteration in at least one target gene selected from ROS1, NTRK1, NTRK2, and NTRK3) an effective amount (e.g., an initial dose of 600 mg per day) of entrectinib; monitoring adverse events (e.g., hepatotoxicity) in the patient, and where the severity of the adverse event of Grade 2 or higher is observed in the patient, modifying/reducing the dosage of entrectinib (e.g., where a Grade 4 adverse event is observed, reducing the dosage to zero or suspending administration of entrectinib; or where a Grade 3 adverse event is observed, withholding entrectinib administration until recovery to less than or equal to Grade 1 or to baseline, and optionally where the recovery occurs within 4 weeks, resuming administering entrectinib at the initial dose, or optionally where recurrent Grade 3 adverse event is observed within 4 weeks, resuming administering entrectinib at a reduced dose (e.g., 400 mg or 200 mg per day)). Adverse events include, but are not limited to, congestive heart failure, central nervous system effects, hepatotoxicity, hyperuricemia, QT interval prolongation, vision disorder, and anemia or neutropenia. Monitoring adverse events may include, but is not limited to, one of more of (1) monitoring the patient for clinical signs and symptoms of congestive heart failure (CHF), including shortness of breath and edema, optionally obtaining a MRI or cardiac biopsy to make a diagnosis of CHF; (2) monitoring the patient for one or more CNS adverse reactions; (3) monitoring the patient for one or more signs or symptoms of skeletal fracture; (4) monitoring the patient with liver tests, including ALT and AST (e.g., every 2 weeks during the first month of administration, then monthly thereafter, and as clinically indicated); (5) monitoring the patient for symptoms of liver problems (e.g., loss of appetite, nausea or vomiting, or pain on the upper right side of the stomach area); (6) assessing serum uric acid levels periodically and monitoring the patient for signs and symptoms of hyperuricemia; (7) assessing QT interval and electrolytes; (8) monitoring the patient for a new vision disorder; and (9) monitoring the patient for anemia or neutropenia. In some embodiments, the initial dose is 600 mg, 500 mg, or 400 mg. In some embodiments, the reduced dose is 200 mg, 300 mg, or 400 mg. In some embodiments, the initial dose is 600 mg and the reduced dose is 400 mg or 200 mg. In some embodiments, patient recovery to less than or equal to Grade 1 severity of adverse reaction includes patient recovery to baseline.

In some embodiments, a method of managing adverse reactions of the administration of entrectinib in a patient comprises assessing left ventricular ejection fraction (LVEF) of the patient prior to administering entrectinib to the patient, wherein the patient has symptoms or known risk factors for congestive heart failure (CHF). In some embodiments, the method comprises monitoring the patient for clinical signs and symptoms of CHF, including shortness of breath and edema. In some embodiments, the method further comprises obtaining a MRI or cardiac biopsy to make a diagnosis of CHF. In some embodiments, if the patient experiences newly onset CHF or worsening CHF, then the method further comprises withholding entrectinib, instituting appropriate medical management, and reassessing LVEF and severity of CHF. In further embodiments, the method further comprises resuming entrectinib at a reduced dose upon patient recovery to baseline or permanently discontinuing administration of entrectinib.

In some embodiments, a method of managing adverse reactions of administration of entrectinib in a patient comprises:
assessing left ventricular ejection fraction (LVEF) of the patient prior to administering entrectinib to the patient;
initiating administration of entrectinib to the patient;
monitoring the patient for clinical signs and symptoms of congestive heart failure (CHF), including shortness of breath and edema;
optionally obtaining a MRI or cardiac biopsy to make a diagnosis of CHF; and
continuing to administer entrectinib to the patient in the absence of adverse reaction in the patient or until cancer progression; or
if the patient experiences Grade 2 or 3 severity of CHF, withholding administration of entrectinib, instituting appropriate medical management, reassessing LVEF and severity of CHF, and resuming administration of entrectinib at a reduced dose upon patient recovery to less than or equal to Grade 1 severity of CHF or permanently discontinuing administration of entrectinib if the patient experiences Grade 4 severity of CHF.

In some embodiments, the severity of CHF is as defined by National Cancer Institute Common Terminology Criteria for Adverse Events (NCI CTCAE) version 4.0, see website at ctep.cancer.gov/protocolDevelopment/electronic_applications/ctc.htm#ctc_40.

Non-limiting examples of signs and symptoms of CHF include persistent coughing or wheezing, trouble breathing when lying down, sudden weight gain, increasing shortness of breath, tiredness/weakness/fatigue, and swelling in ankles, feet, or legs.

In some embodiments, a method of managing adverse reactions of the administration of entrectinib in a patient comprises advising the patient of a risk of one or more central nervous system adverse reactions from the administration of entrectinib. In some embodiments, the one or more central nervous system adverse reactions are selected from cognitive impairment, mood disorders, dizziness, and sleep disturbances. In some embodiments, the method further comprises advising the patient not to drive or operate hazardous machinery if the patient experiences one or more central nervous system adverse reactions from the administration of entrectinib. In some embodiments, the method further comprises withholding entrectinib and then resuming administration of entrectinib at the initial dose or a reduced dose upon improvement of the one or more central nervous system adverse reactions or permanently discontinuing administration of entrectinib based on the severity of the one or more central nervous system adverse reactions.

In some embodiments, a method of managing adverse reactions of administration of entrectinib in a patient comprises:
prior to the administration of entrectinib to the patient,
   advising the patient of a risk of one or more central nervous system (CNS) adverse reactions from the administration of entrectinib; and
   advising the patient not to drive or operate hazardous machinery if the patient experiences one or more CNS adverse reactions from the administration of entrectinib;
initiating administration of entrectinib to the patient with an initial dose;
monitoring the patient for one or more CNS adverse reactions; and
continuing to administer entrectinib to the patient in the absence of adverse reaction in the patient or until cancer progression; or
if the patient experiences Grade 2 severity of CNS adverse reaction, withholding administration of entrectinib and resuming administration of entrectinib at the initial dose or at a reduced dose upon patient recovery to less than or equal to Grade 1 severity of CNS adverse reaction; or
if the patient experiences Grade 3 severity of CNS adverse reaction, withholding administration of entrectinib and resuming administration of entrectinib at the reduced dose upon patient recovery to less than or equal to Grade 1 severity of CNS adverse reaction; or
if the patient experiences Grade 4 severity of CNS adverse reaction, permanently discontinuing administration of entrectinib.

In some embodiments, the severity of CNS adverse reaction is as defined by National Cancer Institute Common Terminology Criteria for Adverse Events (NCI CTCAE) version 4.0.

Non-limiting examples of signs and symptoms of CNS adverse reactions include dizziness, changes in mood, cognitive impairment, hallucinations, and problems with concentration, attention, memory, and sleep. Non-limiting examples of cognitive changes, cognitive disorders, or cognitive impairment include confusion, mental status changes, memory impairment, and hallucinations.

In some embodiments, a method of managing adverse reactions of administration of entrectinib in a patient comprises:
prior to the administration of entrectinib to the patient,
   advising the patient of a potential for cognitive change from the administration of entrectinib; and
   advising the patient not to drive or operate hazardous machinery if the patient experiences one or more symptoms of cognitive change from the administration of entrectinib;
initiating administration of entrectinib to the patient with an initial dose;
monitoring the patient for a cognitive disorder; and
continuing to administer entrectinib to the patient in the absence of adverse reaction in the patient or until cancer progression; or
if the patient experiences an event of at least Grade 2 severity of cognitive disorder, withholding administration of entrectinib until patient recovery to less than or equal to Grade 1 severity of cognitive disorder and resuming administration of entrectinib at a reduced dose; or
if the patient experiences a recurrence of the event of at least Grade 2 severity of cognitive disorder, administering entrectinib at a further reduced dose; or
if the patient experiences prolonged, severe, or intolerable events of cognitive disorder, permanently discontinuing administration of entrectinib.

In some embodiments, the severity of cognitive disorder is as defined by National Cancer Institute Common Terminology Criteria for Adverse Events (NCI CTCAE) version 4.0.

In some embodiments, a method of managing adverse reactions of the administration of entrectinib in a patient comprises withholding entrectinib until patient recovery to less than or equal to Grade 1 severity of transaminase elevation. In further embodiments, the administration of entrectinib is resumed without change in dose upon patient recovery within 4 weeks to less than or equal to Grade 1 severity of transaminase elevation, or the administration of entrectinib is resumed at a reduced dose for recurrent Grade 3 severity of hepatotoxicity that resolve within 4 weeks, or the administration of entrectinib to the patient is permanently discontinued if Grade 3 severity of transaminase elevation does not resolve within 4 weeks. In other embodiments, the administration of entrectinib is resumed without change in dose upon patient recovery within 4 weeks to less than or equal to Grade 1 severity of transaminase elevation, or the administration of entrectinib to the patient permanently is discontinued if Grade 4 severity of transaminase elevation does not resolve within 4 weeks or recurs. In some embodiments, a method of managing adverse reactions of the administration of entrectinib in a patient comprises permanently discontinuing administration of entrectinib if the patient exhibits ALT or AST elevation greater than 3 times ULN with total bilirubin elevation being greater than 2 times ULN in the absence of cholestasis or hemolysis.

In some embodiments, a method of managing adverse reactions of administration of entrectinib in a patient comprises:
initiating administration of entrectinib to the patient with an initial dose;
monitoring the patient for transaminase elevation; and
continuing to administer entrectinib to the patient in the absence of adverse reaction in the patient or until cancer progression; or
if the patient exhibits Grade 3 severity of transaminase elevation, withholding administration of entrectinib until patient recovery to less than or equal to Grade 1 severity of transaminase elevation; and resuming administration of entrectinib to the patient without change in dose upon patient recovery within 4 weeks to less than or equal to Grade 1 severity of transaminase elevation, or resuming administration of entrectinib at a reduced dose for recurrent Grade 3 severity of hepatotoxicity that resolve within 4 weeks, or permanently discontinuing administration of entrectinib to the patient if Grade 3 severity of transaminase elevation does not resolve within 4 weeks; or
if the patient exhibits Grade 4 severity of transaminase elevation, withholding administration of entrectinib until patient recovery to less than or equal to Grade 1 severity of transaminase elevation; and resuming administration of entrectinib to the patient without change in dose upon patient recovery within 4 weeks to less than or equal to Grade 1 severity of transaminase elevation, or permanently discontinuing administration of entrectinib to the patient if Grade 4 severity of transaminase elevation does not resolve within 4 weeks or recurs; or
if the patient exhibits ALT or AST elevation greater than 3 times the upper limit of normal

(ULN) with total bilirubin elevation being greater than 2 times ULN in the absence of cholestasis or hemolysis, permanently discontinuing administration of entrectinib to the patient.

In some embodiments, the severity of transaminase elevation is as defined by National Cancer Institute Common Terminology Criteria for Adverse Events (NCI CTCAE) version 4.0.

In some embodiments, a method of managing adverse reactions of the administration of entrectinib in a patient comprises promptly evaluating the patient with one or more signs or symptoms of skeletal fracture. In some embodiments, the one or more signs or symptoms of skeletal fracture are selected from pain, changes in mobility, deformity, and any combination thereof. In some embodiments, the one or more signs or symptoms of skeletal fracture are selected from pain, abnormal gait, changes in mobility, deformity, and any combination thereof.

In some embodiments, a method of managing adverse reactions of administration of entrectinib in a patient comprises:
initiating administration of entrectinib to the patient;
monitoring the patient for one or more signs or symptoms of skeletal fracture;
if the patient exhibits one or more signs or symptoms of skeletal fracture, promptly evaluating the patient and instituting appropriate medical management; and
continuing to administer entrectinib to the patient in the absence of additional adverse reaction in the patient or until cancer progression.

In some embodiments, a method of managing adverse reactions of the administration of entrectinib in a patient comprises monitoring liver tests, including ALT and AST, every 2 weeks during the first month of treatment, then monthly thereafter, and as clinically indicated. In some embodiments, the method further comprises withholding entrectinib and then resuming administration of entrectinib at the initial dose or a reduced dose upon improvement of hepatotoxicity or permanently discontinuing administration of entrectinib based on the severity of the hepatotoxicity.

In some embodiments, a method of managing adverse reactions of administration of entrectinib in a patient comprises:
initiating administration of entrectinib to the patient with an initial dose;
monitoring the patient with liver tests, including ALT and AST, every 2 weeks during the first month of administration, then monthly thereafter, and as clinically indicated; and
continuing to administer entrectinib to the patient in the absence of adverse reaction in the patient or until cancer progression; or
if the patient experiences Grade 3 severity of hepatotoxicity, withholding administration of entrectinib and resuming administration of entrectinib at the initial dose upon patient recovery within 4 weeks to less than or equal to Grade 1 severity of hepatotoxicity, or resuming administration of entrectinib at a reduced dose for recurrent Grade 3 severity of hepatotoxicity that resolve within 4 weeks; or permanently discontinuing administration of entrectinib if Grade 3 severity of hepatotoxicity does not resolve within 4 weeks; or
if the patient experiences Grade 4 severity of hepatotoxicity, withholding administration of entrectinib and resuming administration of entrectinib at the reduced dose upon patient recovery within 4 weeks to less than or equal to Grade 1 severity of hepatotoxicity, or permanently discontinuing administration of entrectinib if Grade 4 severity of hepatotoxicity does not resolve within 4 weeks or recurs; or
if the patient exhibits ALT or AST greater than 3 times the upper limit of normal (ULN) with concurrent total bilirubin greater than 1.5 times ULN in the absence of cholestasis or hemolysis, permanently discontinuing administration of entrectinib.

In some embodiments, the severity of hepatotoxicity is as defined by National Cancer Institute Common Terminology Criteria for Adverse Events (NCI CTCAE) version 4.0.

In some embodiments, symptoms of liver problems include, but are not limited to, loss of appetite, nausea or vomiting, or pain on the upper right side of the stomach area.

In some embodiments, a method of managing adverse reactions of the administration of entrectinib in a patient comprises assessing serum uric acid levels prior to initiating administration of entrectinib and periodically during administration. In some embodiments, the method further comprises monitoring the patient for signs and symptoms of hyperuricemia. In some embodiments, the method further comprises initiating treatment with urate-lowering medications as clinically indicated and withholding entrectinib for signs and symptoms of hyperuricemia. In further embodiments, the method further comprises resuming administration of entrectinib at the initial dose or a reduced dose upon improvement of signs or symptoms based on severity of hyperuricemia.

In some embodiments, a method of managing adverse reactions of administration of entrectinib in a patient comprises:
assessing serum uric acid levels in the patient prior to initiating administration of entrectinib;
initiating administration of entrectinib to the patient with an initial dose;
reassessing serum uric acid levels periodically during administration of entrectinib and monitoring the patient for signs and symptoms of hyperuricemia; and
continuing to administer entrectinib to the patient in the absence of adverse reaction in the patient or until cancer progression; or
if the patient is symptomatic of hyperuricemia or experiences Grade 4 hyperuricemia, initiating treatment with urate-lowering medications as clinically indicated, withholding entrectinib and resuming administration of entrectinib at the initial dose or at a reduced dose upon improvement of signs or symptoms of hyperuricemia.

In some embodiments, the severity of hyperuricemia is as defined by National Cancer Institute Common Terminology Criteria for Adverse Events (NCI CTCAE) version 4.0.

In some embodiments, a method of managing adverse reactions of the administration of entrectinib in a patient comprises monitoring the patient who already has or who is at significant risk of developing QTc interval prolongation, such as, but not limited to, a patient with known long QT syndromes, a patient with clinically significant bradyarrhythmias, severe or uncontrolled heart failure, or a patient taking other medicinal products associated with QT prolongation. In some embodiments, the method further comprises assessing QT interval and electrolytes at baseline and periodically during administration of entrectinib, adjusting frequency based upon risk factors such as congestive heart failure, electrolyte abnormalities, or concomitant medications known to prolong the QTc interval. In some embodiments, based on the severity of QTc interval prolongation, the method further comprises withholding entrectinib and resuming administration of entrectinib at the initial dose or at a reduced dose or permanently discontinuing administration of entrectinib.

In some embodiments, a method of managing adverse reactions of administration of entrectinib in a patient comprises:
initiating administration of entrectinib to the patient with an initial dose;
monitoring the patient for congestive heart failure; and
continuing to administer entrectinib to the patient in the absence of adverse reaction in the patient or until cancer progression; or
if the patient exhibits Grade 2 or Grade 3 severity of congestive heart failure, withholding administration of entrectinib until patient recovery to less than or equal to Grade 1 severity of congestive heart failure; and resuming administration of entrectinib at a reduced dose; or
if the patient exhibits Grade 4 severity of congestive heart failure, withholding administration of entrectinib until patient recovery to less than or equal to Grade 1 severity of congestive heart failure; and resuming administration of entrectinib at a reduced dose or permanently discontinuing administration of entrectinib to the patient.

In some embodiments, a method of managing adverse reactions of administration of entrectinib in a patient comprises:
initiating administration of entrectinib to the patient with an initial dose;
monitoring the patient for congestive heart failure; and
continuing to administer entrectinib to the patient in the absence of adverse reaction in the patient or until cancer progression; or
if the patient exhibits Grade 2 or Grade 3 severity of congestive heart failure, withholding administration of entrectinib until patient recovery to less than or equal to Grade 1 severity of congestive heart failure; and resuming administration of entrectinib at a reduced dose; or
if the patient exhibits Grade 4 severity of congestive heart failure, permanently discontinuing administration of entrectinib to the patient.

In some embodiments, the severity of congestive heart failure is as defined by National Cancer Institute Common Terminology Criteria for Adverse Events (NCI CTCAE) version 4.0.

In some embodiments, a method of managing adverse reactions of administration of entrectinib in a patient comprises:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc greater than 500 ms, withholding entrectinib until QTc interval recovers to baseline and resuming administration of entrectinib at the initial dose or at a reduced dose; or
if the patient exhibits Torsade de pointes, polymorphic ventricular tachycardia, or signs or symptoms of serious arrhythmia, permanently discontinuing administration of entrectinib.

In some embodiments, administration of entrectinib is resumed at the initial dose if factors that cause QT prolongation are identified and corrected. In some embodiments, administration of entrectinib is resumed at the reduced dose if other factors that cause QT prolongation are not identified.

In some embodiments, a method of managing adverse reactions of administration of entrectinib in a patient comprises:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc of 481 to 500 ms, withholding entrectinib until QTc interval recovers to baseline and resuming administration of entrectinib at the initial dose; or
if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or
if the patient exhibits Torsade de pointes, polymorphic ventricular tachycardia, or signs or symptoms of serious arrhythmia, permanently discontinuing administration of entrectinib.

In some embodiments, a method of managing adverse reactions of the administration of entrectinib in a patient comprises withholding entrectinib until improvement or stabilization of new visual changes that interfere with activities of daily living and resuming administration of entrectinib at the initial dose or at a reduced dose.

In some embodiments, a method of managing adverse reactions of administration of entrectinib in a patient comprises:
initiating administration of entrectinib to the patient with an initial dose;
monitoring the patient for a new vision disorder; and
continuing to administer entrectinib to the patient in the absence of adverse reaction in the patient or until cancer progression; or
if the patient experiences Grade 2 or higher severity of vision disorder, withholding entrectinib until improvement or stabilization of the vision disorder, and resuming administration of entrectinib at the initial dose or at a reduced dose.

In some embodiments, the severity of vision disorder is as defined by National Cancer Institute Common Terminology Criteria for Adverse Events (NCI CTCAE) version 4.0.

In some embodiments, the vision disorder is selected from double vision, blurry vision, new or increased floaters, flashes of light, light sensitivity, and a combination of two or more thereof. In some embodiments, the vision disorder is selected from blurred vision, photophobia, diplopia, visual impairment, photopsia, cataract, vitreous floaters, and a combination of two or more thereof.

In some embodiments, a method of managing adverse reactions of the administration of entrectinib in a patient comprises withholding entrectinib until patient recovery to less than or equal to Grade 2 severity anemia or neutropenia.

In some embodiments, a method of managing adverse reactions of administration of entrectinib in a patient comprises:
initiating administration of entrectinib to the patient with an initial dose;
monitoring the patient for anemia or neutropenia; and
continuing to administer entrectinib to the patient in the absence of adverse reaction in the patient or until cancer progression; or
if the patient experiences Grade 3 or Grade 4 severity of anemia or neutropenia, withholding entrectinib and resuming administration of entrectinib at the initial dose or at a reduced dose upon patient recovery to Grade 2 or less severity of anemia or neutropenia.

In some embodiments, the severity of anemia or neutropenia is as defined by National Cancer Institute Common Terminology Criteria for Adverse Events (NCI CTCAE) version 4.0.

In some embodiments, a method of managing adverse reactions of administration of entrectinib in a patient comprises
prior to the administration of entrectinib to the patient,
   advising the patient of a potential for embryo-fetal toxicity;
initiating administration of entrectinib to the patient; and
continuing to administer entrectinib to the patient in the absence of adverse reaction in the patient or until cancer progression; or
if the patient becomes pregnant, permanently discontinuing administration of entrectinib.

In some embodiments, a method of managing adverse reactions of the administration of entrectinib in a patient comprises withholding entrectinib until a Grade 3 or Grade 4 adverse reaction resolves or improves to recovery or improvement to Grade 1 or less.

In some embodiments, a method of managing adverse reactions of administration of entrectinib in a patient comprises:
initiating administration of entrectinib to the patient with an initial dose;
monitoring the patient for adverse reaction; and
continuing to administer entrectinib to the patient in the absence of adverse reaction in the patient or until cancer progression; or
if the patient experiences Grade 3 or Grade 4 severity of adverse reaction, withholding entrectinib and resuming administration of entrectinib at the initial dose or at a reduced dose upon patient recovery to Grade 1 or less severity of adverse reaction or permanently discontinuing administration of entrectinib if adverse reaction does not resolve within 4 weeks or Grade 4 severity adverse reactions recur.

In some embodiments, the severity of an adverse reaction is as defined by National Cancer Institute Common Terminology Criteria for Adverse Events (NCI CTCAE) version 4.0.

In some embodiments, provided herein is a method of treating cancer in a patient in need thereof, the method comprising:
assessing left ventricular ejection fraction (LVEF) of the patient prior to administering entrectinib to the patient;
initiating administration of entrectinib to the patient;
monitoring the patient for clinical signs and symptoms of congestive heart failure (CHF), including shortness of breath and edema;
optionally obtaining a MRI or cardiac biopsy to make a diagnosis of CHF; and
continuing to administer entrectinib to the patient in the absence of adverse reaction in the patient or until cancer progression; or
if the patient experiences Grade 2 or 3 severity of CHF, withholding administration of entrectinib, instituting appropriate medical management, reassessing LVEF and severity of CHF, and resuming administration of entrectinib at a reduced dose upon patient recovery to less than or equal to Grade 1 severity of CHF or permanently discontinuing administration of entrectinib if the patient experiences Grade 4 severity of CHF.

In some embodiments, provided herein is a method of treating cancer in a patient in need thereof, the method comprising:
prior to administration of entrectinib to the patient,
   advising the patient of a risk of one or more central nervous system (CNS) adverse reactions from the administration of entrectinib; and
   advising the patient not to drive or operate hazardous machinery if the patient experiences one or more CNS adverse reactions from the administration of entrectinib;
initiating administration of entrectinib to the patient with an initial dose;
monitoring the patient for one or more CNS adverse reactions; and
continuing to administer entrectinib to the patient in the absence of adverse reaction in the patient or until cancer progression; or
if the patient experiences Grade 2 severity of CNS adverse reaction, withholding administration of entrectinib and resuming administration of entrectinib at the initial dose or at a reduced dose upon patient recovery to less than or equal to Grade 1 severity of CNS adverse reaction; or
if the patient experiences Grade 3 severity of CNS adverse reaction, withholding administration of entrectinib and resuming administration of entrectinib at the reduced dose upon patient recovery to less than or equal to Grade 1 severity of CNS adverse reaction; or
if the patient experiences Grade 4 severity of CNS adverse reaction, permanently discontinuing administration of entrectinib.

In some embodiments, provided herein is a method of treating cancer in a patient in need thereof, the method comprising:
prior to the administration of entrectinib to the patient,
   advising the patient of a potential for cognitive change from the administration of entrectinib; and
   advising the patient not to drive or operate hazardous machinery if the patient experiences one or more symptoms of cognitive change from the administration of entrectinib;
initiating administration of entrectinib to the patient with an initial dose;
monitoring the patient for a cognitive disorder; and
continuing to administer entrectinib to the patient in the absence of adverse reaction in the patient or until cancer progression; or
if the patient experiences an event of at least Grade 2 severity of cognitive disorder, withholding administration of entrectinib until patient recovery to less than or equal to Grade 1 severity of cognitive disorder and resuming administration of entrectinib at a reduced dose; or
if the patient experiences a recurrence of the event of at least Grade 2 severity of cognitive disorder, administering entrectinib at a further reduced dose; or
if the patient experiences prolonged, severe, or intolerable events of cognitive disorder, permanently discontinuing administration of entrectinib.

In some embodiments, provided herein is a method of treating cancer in a patient in need thereof, the method comprising:
initiating administration of entrectinib to the patient with an initial dose;
monitoring the patient for transaminase elevation; and
continuing to administer entrectinib to the patient in the absence of adverse reaction in the patient or until cancer progression; or
if the patient exhibits Grade 3 severity of transaminase elevation, withholding administration of entrectinib until patient recovery to less than or equal to Grade 1 severity of transaminase elevation; and resuming administration of entrectinib to the patient without change in dose upon patient recovery within 4 weeks to less than or equal to Grade 1 severity of transaminase elevation, or resuming administration of entrectinib at a reduced dose for recurrent Grade 3 severity of hepatotoxicity that resolve within 4 weeks, or permanently discontinuing administration of entrectinib to the patient if Grade 3 severity of transaminase elevation does not resolve within 4 weeks; or
if the patient exhibits Grade 4 severity of transaminase elevation, withholding administration of entrectinib until patient recovery to less than or equal to Grade 1 severity of transaminase elevation; and resuming administration of entrectinib to the patient without change in dose upon patient recovery within 4 weeks to less than or equal to Grade 1 severity of transaminase elevation, or permanently discontinuing administration of entrectinib to the patient if Grade 4 severity of transaminase elevation does not resolve within 4 weeks or recurs; or
if the patient exhibits ALT or AST elevation greater than 3 times the upper limit of normal (ULN) with total bilirubin elevation being greater than 2 times ULN in the absence of cholestasis or hemolysis, permanently discontinuing administration of entrectinib to the patient.

In some embodiments, provided herein is a method of treating cancer in a patient in need thereof, the method comprising:
initiating administration of entrectinib to the patient;
monitoring the patient for one or more signs or symptoms of skeletal fracture;
if the patient exhibits one or more signs or symptoms of skeletal fracture, promptly evaluating the patient and instituting appropriate medical management; and
continuing to administer entrectinib to the patient in the absence of additional adverse reaction in the patient or until cancer progression.

In some embodiments, provided herein is a method of treating cancer in a patient in need thereof, the method comprising:
initiating administration of entrectinib to the patient with an initial dose;
monitoring the patient with liver tests, including ALT and AST, every 2 weeks during the first month of administration, then monthly thereafter, and as clinically indicated; and
continuing to administer entrectinib to the patient in the absence of adverse reaction in the patient or until cancer progression; or
if the patient experiences Grade 3 severity of hepatotoxicity, withholding administration of entrectinib and resuming administration of entrectinib at the initial dose upon patient recovery within 4 weeks to less than or equal to Grade 1 severity of hepatotoxicity, or resuming administration of entrectinib at a reduced dose for recurrent Grade 3 severity of hepatotoxicity that resolve within 4 weeks; or permanently discontinuing administration of entrectinib if Grade 3 severity of hepatotoxicity does not resolve within 4 weeks; or
if the patient experiences Grade 4 severity of hepatotoxicity, withholding administration of entrectinib and resuming administration of entrectinib at the reduced dose upon patient recovery within 4 weeks to less than or equal to Grade 1 severity of hepatotoxicity, or permanently discontinuing administration of entrectinib if Grade 4 severity of hepatotoxicity does not resolve within 4 weeks or recurs; or
if the patient exhibits ALT or AST greater than 3 times the upper limit of normal (ULN) with concurrent total bilirubin greater than 1.5 times ULN in the absence of cholestasis or hemolysis, permanently discontinuing administration of entrectinib.

In some embodiments, provided herein is a method of treating cancer in a patient in need thereof, the method comprising:
assessing serum uric acid levels in the patient prior to initiating administration of entrectinib;
initiating administration of entrectinib to the patient with an initial dose;
reassessing serum uric acid levels periodically during administration of entrectinib and monitoring the patient for signs and symptoms of hyperuricemia; and
continuing to administer entrectinib to the patient in the absence of adverse reaction in the patient or until cancer progression; or
if the patient is symptomatic of hyperuricemia or experiences Grade 4 hyperuricemia, initiating treatment with urate-lowering medications as clinically indicated, withholding entrectinib and resuming administration of entrectinib at the initial dose or at a reduced dose upon improvement of signs or symptoms of hyperuricemia.

In some embodiments, provided herein is a method of treating cancer in a patient in need thereof, the method comprising:
initiating administration of entrectinib to the patient with an initial dose;
monitoring the patient for congestive heart failure; and
continuing to administer entrectinib to the patient in the absence of adverse reaction in the patient or until cancer progression; or
if the patient exhibits Grade 2 or Grade 3 severity of congestive heart failure, withholding administration of entrectinib until patient recovery to less than or equal to Grade 1 severity of congestive heart failure; and resuming administration of entrectinib at a reduced dose; or
if the patient exhibits Grade 4 severity of congestive heart failure, withholding administration of entrectinib until patient recovery to less than or equal to Grade 1 severity of congestive heart failure; and resuming administration of entrectinib at a reduced dose or permanently discontinuing administration of entrectinib to the patient.

In some embodiments, provided herein is a method of treating cancer in a patient in need thereof, the method comprising:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc greater than 500 ms, withholding entrectinib until QTc interval recovers to baseline and resuming administration of entrectinib at the initial dose or at a reduced dose; or
if the patient exhibits Torsade de pointes, polymorphic ventricular tachycardia, or signs or symptoms of serious arrhythmia, permanently discontinuing administration of entrectinib.

In some embodiments, provided herein is a method of treating cancer in a patient in need thereof, the method comprising:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc of 481 to 500 ms, withholding entrectinib until QTc interval recovers to baseline and resuming administration of entrectinib at the initial dose; or
if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or
if the patient exhibits Torsade de pointes, polymorphic ventricular tachycardia, or signs or symptoms of serious arrhythmia, permanently discontinuing administration of entrectinib.

In some embodiments, provided herein is a method of treating cancer in a patient in need thereof, the method comprising:
initiating administration of entrectinib to the patient with an initial dose;
monitoring the patient for a new vision disorder; and
continuing to administer entrectinib to the patient in the absence of adverse reaction in the patient or until cancer progression; or
if the patient experiences Grade 2 or higher severity of vision disorder, withholding entrectinib until improvement or stabilization of the vision disorder, and resuming administration of entrectinib at the initial dose or at a reduced dose.

In some embodiments, provided herein is a method of treating cancer in a patient in need thereof, the method comprising:
initiating administration of entrectinib to the patient with an initial dose;
monitoring the patient for anemia or neutropenia; and
continuing to administer entrectinib to the patient in the absence of adverse reaction in the patient or until cancer progression; or
if the patient experiences Grade 3 or Grade 4 severity of anemia or neutropenia, withholding entrectinib and resuming administration of entrectinib at the initial dose or at a reduced dose upon patient recovery to Grade 2 or less severity of anemia or neutropenia.

In some embodiments, provided herein is a method of treating cancer in a patient in need thereof, the method comprising:
prior to the administration of entrectinib to the patient,
   advising the patient of a potential for embryo-fetal toxicity;
initiating administration of entrectinib to the patient; and
continuing to administer entrectinib to the patient in the absence of adverse reaction in the patient or until cancer progression; or
if the patient becomes pregnant, permanently discontinuing administration of entrectinib.

In some embodiments, provided herein is a method of treating cancer in a patient in need thereof, the method comprising:
initiating administration of entrectinib to the patient with an initial dose;
monitoring the patient for adverse reaction; and
continuing to administer entrectinib to the patient in the absence of adverse reaction in the patient or until cancer progression; or
if the patient experiences Grade 3 or Grade 4 severity of adverse reaction, withholding entrectinib and resuming administration of entrectinib at the initial dose or at a reduced dose upon patient recovery to Grade 1 or less severity of adverse reaction or permanently discontinuing administration of entrectinib if adverse reaction does not resolve within 4 weeks or Grade 4 severity adverse reactions recur.

In some embodiments, the initial dose is 600 mg once daily. In some embodiments, the initial dose is 500 mg once daily. In some embodiments, the initial dose is 400 mg once daily.

In some embodiments, the patient is an adult patient. In some embodiments, the patient is an adult patient, and the initial dose is 600 mg once daily. In some embodiments, the patient is a pediatric patient who is 12 years or older with BSA greater than 1.50 m², and the initial dose is 600 mg once daily. In some embodiments, the patient is a pediatric patient who is 12 years or older with BSA of 1.11 to 1.50 m², and the initial dose is 500 mg once daily. In some embodiments, the patient is a pediatric patient who is 12 years or older with BSA of 0.91 to 1.10 m², and the initial dose is 400 mg once daily.

In some embodiments, the patient is a pediatric patient. In some embodiments, the patient is a pediatric patient who has the ability to swallow capsules, and the daily dose is 300 mg/m². In some embodiments, the patient is a pediatric patient with BSA of 0.43 to 0.50 m², and the daily dose is 100 mg. In some embodiments, the patient is a pediatric patient with BSA of 0.51 to 0.80 m², and the daily dose is 200 mg. In some embodiments, the patient is a pediatric patient with BSA of 0.81 to 1.10 m², and the daily dose is 300 mg. In some embodiments, the patient is a pediatric patient with BSA of 1.11 to 1.50 m², and the daily dose is 400 mg. In some embodiments, the patient is a pediatric patient with BSA of ≥1.51 m², and the daily dose is 500 mg. In other embodiments, the patient is a pediatric patient with BSA of ≥1.51 m², and the daily dose is 600 mg.

In some embodiments, the patient is a pediatric patient with BSA of 0.91 to 1.10 m², and the daily dose is 400 mg. In some embodiments, the patient is a pediatric patient with BSA of 1.11 to 1.50 m², and the daily dose is 500 mg. In some embodiments, the patient is a pediatric patient with BSA of >1.50 m², and the daily dose is 600 mg.

Some embodiments include any of the methods described herein, wherein any of the pharmaceutical compositions provided herein that comprise entrectinib are administered to a subject in an amount such that the amount of entrectinib the subject receives is about 50 mg, about 100 mg, about 150 mg, about 200 mg, about 250 mg, about 300 mg, about 350 mg, about 400 mg, about 450 mg, about 500 mg, about 550 mg, or about 600 mg. In some embodiments, the amount of entrectinib the subject receives is between about 50 mg and about 600 mg. In some embodiments, the amount is in a range defined by an lower limit of about 50 mg, about 100 mg, about 150 mg, about 200 mg, about 250 mg, about 300 mg, about 350 mg, about 400 mg, about 450 mg, about 500 mg, or about 550 mg, and a upper limit of about 100 mg, about 150 mg, about 200 mg, about 250 mg, about 300 mg, about 350 mg, about 400 mg, about 450 mg, about 500 mg, about 550 mg, or about 600 mg, where the upper limit is higher than the lower limit. In some embodiments, the amount of entrectinib the subject receives is about 50 mg. In some embodiments, the amount of entrectinib the subject receives is about 100 mg. In some embodiments, the amount of entrectinib the subject receives is about 150 mg. In some embodiments, the amount of entrectinib the subject receives is about 200 mg. In some embodiments, the amount of entrectinib the subject receives is about 300 mg. In some embodiments, the amount of entrectinib the subject receives is about 400 mg. In some embodiments, the amount of entrectinib the subject receives is about 500 mg. In some embodiments, the amount of entrectinib the subject receives is about 600 mg.

In some embodiments, the subject receives an amount of entrectinib once a day, twice a day, or three times a day. In some embodiments, the subject receives an amount of entrectinib every other day, every two days, or every three days. In some embodiments, the subject receives an amount of entrectinib once per week.

Some embodiments include any of the methods described herein, wherein any of the pharmaceutical compositions provided herein that comprise entrectinib are administered to a subject once per day in an amount such that the amount of entrectinib the subject receives per day is about 50 mg, about 100 mg, about 150 mg, about 200 mg, about 250 mg, about 300 mg, about 350 mg, about 400 mg, about 450 mg, about 500 mg, about 550 mg, or about 600 mg. In some embodiments, the amount of entrectinib the subject receives once per day is between about 50 mg and about 600 mg. In some embodiments, the amount of entrectinib the subject receives once per day is in a range defined by an lower limit of about 50 mg, about 100 mg, about 150 mg, about 200 mg, about 250 mg, about 300 mg, about 350 mg, about 400 mg, about 450 mg, about 500 mg, or about 550 mg, and a upper limit of about 100 mg, about 150 mg, about 200 mg, about 250 mg, about 300 mg, about 350 mg, about 400 mg, about 450 mg, about 500 mg, about 550 mg, or about 600 mg, where the upper limit is higher than the lower limit. In some embodiments, the amount of entrectinib the subject receives once per day is about 50 mg. In some embodiments, the amount of entrectinib the subject receives once per day is about 100 mg.

In some embodiments, the amount of entrectinib the subject receives once per day is about 150 mg. In some embodiments, the amount of entrectinib the subject receives once per day is about 200 mg. In some embodiments, the amount of entrectinib the subject receives once per day is about 300 mg. In some embodiments, the amount of entrectinib the subject receives once per day is about 400 mg. In some embodiments, the amount of entrectinib the subject receives once per day is about 500 mg. In some embodiments, the amount of entrectinib the subject receives once per day is about 600 mg.

In some embodiments, a first dose reduction of entrectinib for an adult patient is 400 mg, orally administered once daily. In further embodiments, a second dose reduction of entrectinib for an adult patient is 200 mg, orally administered once daily. In still further embodiments, administration of entrectinib is permanently discontinued after two dose reductions for an adult patient. In some embodiments, a starting dose of entrectinib for an adult patient is 600 mg, orally administered once daily; a first dose reduction of entrectinib for an adult patient is 400 mg, orally administered once daily; a second dose reduction of entrectinib for an adult patient is 200 mg, orally administered once daily; and administration of entrectinib is permanently discontinued after two dose reductions if the adult patient cannot tolerate the second dose reduction.

In some embodiments, a first dose reduction of entrectinib for pediatric patients is 100 mg, orally administered once per day for 5 days each week (e.g., Monday, Wednesday, Friday, Saturday, and Sunday). In further embodiments, a second dose reduction of entrectinib for pediatric patients is 100 mg, orally administered once per day for 3 days each week (e.g., Monday, Thursday, and Saturday). In still further embodiments, administration of entrectinib is permanently discontinued after two dose reductions for pediatric patients. In some embodiments, a starting dose of entrectinib for pediatric patients is 100 mg, orally administered once daily; a first dose reduction of entrectinib for pediatric patients is 100 mg, orally administered once per day for 5 days each week (e.g., Monday, Wednesday, Friday, Saturday, and Sunday); a second dose reduction of entrectinib for pediatric patients is 100 mg, orally administered once per day for 3 days each week (e.g., Monday, Thursday, and Saturday); and administration of entrectinib is permanently discontinued after two dose reductions if the pediatric patients cannot tolerate the second dose reduction. In some embodiments, a starting dose of entrectinib for pediatric patients with BSA of 0.43 to 0.50 m² is 100 mg, orally administered once daily; a first dose reduction of entrectinib for pediatric patients is 100 mg, orally administered once per day for 5 days each week (e.g., Monday, Wednesday, Friday, Saturday, and Sunday); a second dose reduction of entrectinib for pediatric patients is 100 mg, orally administered once per day for 3 days each week (e.g., Monday, Thursday, and Saturday); and administration of entrectinib is permanently discontinued after two dose reductions if the pediatric patients cannot tolerate the second dose reduction.

In some embodiments, a first dose reduction of entrectinib for pediatric patients is 200 mg, orally administered once per day for 5 days each week (e.g., Monday, Wednesday, Friday, Saturday, and Sunday). In further embodiments, a second dose reduction of entrectinib for pediatric patients is 100 mg, orally administered once per day for 5 days each week (e.g., Monday, Wednesday, Friday, Saturday, and Sunday). In still further embodiments, administration of entrectinib is permanently discontinued after two dose reductions for pediatric patients. In some embodiments, a starting dose of entrectinib for pediatric patients is 200 mg, orally administered once daily; a first dose reduction of entrectinib for pediatric patients is 200 mg, orally administered once per day for 5 days each week (e.g., Monday, Wednesday, Friday, Saturday, and Sunday); a second dose reduction of entrectinib for pediatric patients is 100 mg, orally administered once per day for 5 days each week (e.g., Monday, Wednesday, Friday, Saturday, and Sunday); and administration of entrectinib is permanently discontinued after two dose reductions if the pediatric patients cannot tolerate the second dose reduction. In some embodiments, a starting dose of entrectinib for pediatric patients with BSA of 0.51 to 0.80 m² is 200 mg, orally administered once daily; a first dose reduction of entrectinib for pediatric patients is 200 mg, orally administered once per day for 5 days each week (e.g., Monday, Wednesday, Friday, Saturday, and Sunday); a second dose reduction of entrectinib for pediatric patients is 100 mg, orally administered once per day for 5 days each week (e.g., Monday, Wednesday, Friday, Saturday, and Sunday); and administration of entrectinib is permanently discontinued after two dose reductions if the pediatric patients cannot tolerate the second dose reduction.

In some embodiments, a first dose reduction of entrectinib for pediatric patients is 200 mg, orally administered once daily. In further embodiments, a second dose reduction of entrectinib for pediatric patients is 100 mg, orally administered once daily. In still further embodiments, administration of entrectinib is permanently discontinued after two dose reductions for pediatric patients. In some embodiments, a starting dose of entrectinib for pediatric patients is 300 mg, orally administered once daily; a first dose reduction of entrectinib for pediatric patients is 200 mg, orally administered once daily; a second dose reduction of entrectinib for pediatric patients is 100 mg, orally administered once daily; and administration of entrectinib is permanently discontinued after two dose reductions if the pediatric patients cannot tolerate the second dose reduction. In some embodiments, a starting dose of entrectinib for pediatric patients with BSA of 0.81 to 1.10 m² is 300 mg, orally administered once daily; a first dose reduction of entrectinib for pediatric patients is 200 mg, orally administered once daily; a second dose reduction of entrectinib for pediatric patients is 100 mg, orally administered once daily; and administration of entrectinib is permanently discontinued after two dose reductions if the pediatric patients cannot tolerate the second dose reduction.

In some embodiments, a first dose reduction of entrectinib for pediatric patients is 300 mg, orally administered once daily. In further embodiments, a second dose reduction of entrectinib for pediatric patients is 200 mg, orally administered once per day for 5 days each week (e.g., Monday, Wednesday, Friday, Saturday, and Sunday). In still further embodiments, administration of entrectinib is permanently discontinued after two dose reductions for pediatric patients. In some embodiments, a starting dose of entrectinib for pediatric patients is 400 mg, orally administered once daily; a first dose reduction of entrectinib for pediatric patients is 300 mg, orally administered once daily; a second dose reduction of entrectinib for pediatric patients is 200 mg, orally administered once per day for 5 days each week (e.g., Monday, Wednesday, Friday, Saturday, and Sunday); and administration of entrectinib is permanently discontinued after two dose reductions if the pediatric patients cannot tolerate the second dose reduction. In some embodiments, a starting dose of entrectinib for pediatric patients with BSA of 1.11 to 1.50 m² is 400 mg, orally administered once daily; a first dose reduction of entrectinib for pediatric patients is 300 mg, orally administered once daily; a second dose reduction of entrectinib for pediatric patients is 200 mg, orally administered once per day for 5 days each week (e.g., Monday, Wednesday, Friday, Saturday, and Sunday); and administration of entrectinib is permanently discontinued after two dose reductions if the pediatric patients cannot tolerate the second dose reduction.

In some embodiments, a first dose reduction of entrectinib for pediatric patients is 400 mg, orally administered once daily. In further embodiments, a second dose reduction of entrectinib for pediatric patients is 200 mg, orally administered once daily. In still further embodiments, administration of entrectinib is permanently discontinued after two dose reductions for pediatric patients. In some embodiments, a starting dose of entrectinib for pediatric patients is 600 mg, orally administered once daily; a first dose reduction of entrectinib for pediatric patients is 400 mg, orally administered once daily; a second dose reduction of entrectinib for pediatric patients is 200 mg, orally administered once daily; and administration of entrectinib is permanently discontinued after two dose reductions if the pediatric patients cannot tolerate the second dose reduction. In some embodiments, a starting dose of entrectinib for pediatric patients with BSA of ≥1.51 m² is 600 mg, orally administered once daily; a first dose reduction of entrectinib for pediatric patients is 400 mg, orally administered once daily; a second dose reduction of entrectinib for pediatric patients is 200 mg, orally administered once daily; and administration of entrectinib is permanently discontinued after two dose reductions if the pediatric patients cannot tolerate the second dose reduction.

In some embodiments, a first dose reduction of entrectinib for adults and pediatric patients 12 years and older with BSA greater than 1.50 m² is 400 mg, orally administered once daily. In further embodiments, a second dose reduction of entrectinib for adults and pediatric patients 12 years and older with BSA greater than 1.50 m² is 200 mg, orally administered once daily. In still further embodiments, administration of entrectinib is permanently discontinued after two dose reductions for adults and pediatric patients 12 years and older with BSA greater than 1.50 m².

In some embodiments, a first dose reduction of entrectinib for adults and pediatric patients 12 years and older with BSA of 1.11 to 1.50 m² is 400 mg, orally administered once daily. In further embodiments, a second dose reduction of entrectinib for adults and pediatric patients 12 years and older with BSA of 1.11 to 1.50 m² is 200 mg, orally administered once daily. In still further embodiments, administration of entrectinib is permanently discontinued after two dose reductions for adults and pediatric patients 12 years and older with BSA of 1.11 to 1.50 m².

In some embodiments, a first dose reduction of entrectinib for adults and pediatric patients 12 years and older with BSA of 0.91 to 1.10 m² is 300 mg, orally administered once daily. In further embodiments, a second dose reduction of entrectinib for adults and pediatric patients 12 years and older with BSA of 0.91 to 1.10 m² is 200 mg, orally administered once daily. In still further embodiments, administration of entrectinib is permanently discontinued after two dose reductions for adults and pediatric patients 12 years and older with BSA of 0.91 to 1.10 m².

In some embodiments, 200 mg of entrectinib is administered orally once daily to an adult or pediatric patient 12 years and older with BSA greater than 1.50 m² if a moderate CYP3A inhibitor is co-administered to the adult or pediatric patient. In some embodiments, 200 mg of entrectinib is administered orally once daily to an adult patient if a moderate CYP3A inhibitor is co-administered to the adult patient. In some embodiments, the co-administration of a moderate CYP3A inhibitor is avoided or limited to 14 days or less.

In some embodiments, 100 mg of entrectinib is administered orally once daily to an adult or pediatric patient 12 years and older with BSA greater than 1.50 m² if a strong CYP3A inhibitor is co-administered to the adult or pediatric patient. In some embodiments, 100 mg of entrectinib is administered orally once daily to an adult patient if a strong CYP3A inhibitor is co-administered to the adult patient. In some embodiments, the co-administration of a strong CYP3A inhibitor is avoided or limited to 14 days or less.

In some embodiments, the co-administration of a moderate or strong CYP3A inhibitor to a pediatric patient is avoided.

In some embodiments, the co-administration of a CYP3A inducer to an adult patient or a pediatric patient is avoided.

After discontinuation of the moderate or strong CYP3A inhibitor for 3 to 5 elimination half-lives, the adult or pediatric patient may resume the initial dose of entrectinib that was taken prior to initiating the CYP3A inhibitor. In some embodiments, the initial dose is 600 mg.

Those of ordinary skill in the art will understand that with respect to pharmaceutical compositions comprising entrectinib provided herein the particular pharmaceutical composition, the dosage, and the number of doses given per day to a mammal requiring such treatment, are all choices within the knowledge of one of ordinary skill in the art and can be determined without undue experimentation.

Accordingly, while certain dose and administration regimens are exemplified herein, these examples in no way limit the dose and administration regimen that may be provided to a subject in practicing the presently disclosed methods.

It is to be noted that dosage values may vary with the type and severity of the condition to be alleviated, and may include single or multiple doses. It is to be further understood that for any particular subject, specific dosage regimens should be adjusted over time according to the subject need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. The embodiments provided herein are intended to encompass intra-subject dose-escalation as determined by the skilled artisan. Determining appropriate dosages and regimens for administration of the chemotherapeutic agent are well-known in the relevant art and would be understood to be encompassed by the skilled artisan once provided the teachings provided herein.

In some embodiments are provided pharmaceutical compositions comprising entrectinib and one or more pharmaceutically acceptable excipients, carriers, and/or diluents.

The pharmaceutically acceptable carrier may comprise a conventional pharmaceutical carrier or excipient. Suitable pharmaceutical carriers include inert diluents or fillers, glidants, lubricants, water and various organic solvents (such as hydrates and solvates). The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. In some embodiments, the excipient comprises pre-gelatinized starch. In some embodiments, the pharmaceutical compositions comprise a glidant. In some embodiments, the pharmaceutical compositions comprise colloidal silicon dioxide. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols.

The pharmaceutical carriers employed may be either solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the inventive compositions may include time-delay or time-release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol, Gelucire or the like, or formulator, such as CMC (carboxy-methylcellulose), PG (propyleneglycol), or PEG (polyethyleneglycol), may be added. Gelucire®, a semi-solid vehicle that protects active ingredients from light, moisture and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g. parenteral or oral administration.

To obtain a stable water-soluble dose form, entrectinib may be dissolved in an aqueous solution of an organic or inorganic acid, such as a 0.3 M solution of succinic acid or citric acid. If a soluble salt form is not available, entrectinib may be dissolved in a suitable co-solvent or combinations of co-solvents. Examples of suitable co-solvents include alcohol, propylene glycol, polyethylene glycol 300, polysorbate 80, glycerin and the like in concentrations ranging from 0 to 60% of the total volume. In an exemplary embodiment, entrectinib is dissolved in DMSO and diluted with water. The pharmaceutical composition may also be in the form of a solution of a salt form of entrectinib in an appropriate aqueous vehicle such as water or isotonic saline or dextrose solution.

Proper formulation is dependent upon the route of administration selected. For injection, entrectinib may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, entrectinib may be formulated by combining it with pharmaceutically acceptable carriers known in the art. Such carriers enable entrectinib to be formulated as tablets, pills, dragees, capsules, powders, granules, liquids, gels, syrups, slurries, suspensions and the like, for oral ingestion by a subject to be treated. Pharmaceutical preparations for oral use can be obtained using a solid excipient in admixture with entrectinib, optionally grinding the resulting mixture, and processing the mixture of granules after adding suitable auxiliaries, if desired, to obtain tablets or dragee cores. Suitable excipients include: fillers such as sugars, comprising isomalt, lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, microcrystalline cellulose or polyvinylpyrrolidone (PVP). In some embodiments, the filler is mannitol, isomalt, hypromellose (hydroxypropylmethylcellulose), or microcrystalline cellulose. In some embodiments, the filler is mannitol. In some embodiments, the filler is isomalt. In some embodiments, the filler is microcrystalline cellulose. In some embodiments, the filler is lactose. In some embodiments, the filler is anhydrous lactose. If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings.

Pharmaceutical preparations that can be used orally include push-fit capsules made of gelatin, as well as soft, sealed capsules made of gelatin and a plasticizer, such as glycerol or sorbitol. The push-fit capsules can contain entrectinib in admixture with fillers such as lactose, binders such as starches, and/or lubricants such as talc or magnesium stearate, and, optionally, stabilizers. In soft capsules, entrectinib may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

In some embodiments, the pharmaceutical composition comprises about 40% w/w to about 50% w/w entrectinib, about 10% w/w to about 15% w/w tartaric acid, about 25% w/w to about 30% w/w lactose, about 3% w/w to about 5% w/w hypromellose (hydroxypropyl methylcellulose), about 2% w/w to about 4% w/w microcrystalline cellulose, about 4% w/w to about 7% w/w crospovidone, about 0.1% w/w to about 1% w/w colloidal silicon dioxide, and about 0.5% w/w to about 2% w/w magnesium stearate. In further embodiments, the lactose is anhydrous lactose.

In some embodiments, the pharmaceutical composition comprises ingredients in the amounts listed in Table 3.

TABLE 3

| | | |
|---|---|---|
| | | Amount per |
| Component | % w/w | Capsule (mg) |

Intragranular Components

| | | |
|---|---|---|
| Entrectinib | 44.44 | 100.00 |
| Lactose Anhydrous | 28.89 | 65.00 |
| Hydroxypropyl Methylcellulose | 4.00 | 9.00 |
| Crospovidone | 2.78 | 6.25 |
| Tartaric Acid | 13.11 | 29.50 |
| Magnesium Stearate | 0.56 | 1.25 |

Extragranular Components

| | | |
|---|---|---|
| Microcrystalline Cellulose | 2.97 | 6.685 |
| Crospovidone | 2.50 | 5.625 |
| Colloidal Silicon Dioxide | 0.25 | 0.565 |
| Magnesium Stearate | 0.50 | 1.125 |
| Total | 100.0 | 225.00 |

In some embodiments, a composition of a capsule is as follows:

TABLE 4

| | | Amount per |
|---|---|---|
| Component | % w/w | Capsule (mg) |
| Capsule, HPMC, Size 2, Yellow | | 1 each |
| Opaque Body and Yellow Opaque Cap | | |
| Titanium Dioxide | 1.4584 | 0.83-0.98 |
| Hypromellose | QSP 100 | QSP 57-65 |
| FDA/E172 Yellow Iron Oxide | 0.2307 | 0.13-0.15 |

QSP = Quantité Suffisante Pour” (Quality Sufficient For)

In some embodiments, the pharmaceutical composition comprises ingredients in the amounts listed below.

TABLE 5

| | | |
|---|---|---|
| | | Amount per |
| Component | % w/w | Capsule (mg) |

Intragranular Components

| | | |
|---|---|---|
| N-[5-(3,5-difluorobenzyl)-1H-indazol-3-yl]- | 44.44 | 200.00 |
| 4-(4-methyl-piperazin-1-yl)-2-(tetrahydro-2H- | | |
| pyran-4-ylamino)-benzamide | | |
| Lactose Anhydrous | 28.89 | 130.00 |
| Hydroxypropyl Methylcellulose | 4.00 | 18.00 |
| Crospovidone | 2.78 | 12.50 |
| Tartaric Acid | 13.11 | 59.00 |
| Magnesium Stearate | 0.56 | 2.50 |

Extragranular Components

| | | |
|---|---|---|
| Microcrystalline Cellulose | 2.97 | 13.37 |
| Crospovidone | 2.50 | 11.25 |
| Colloidal Silicon Dioxide | 0.25 | 1.13 |
| Magnesium Stearate | 0.50 | 2.25 |
| Total | 100.0 | 450.00 |

In some embodiments, a composition of a capsule is as follows:

TABLE 6

| | | Amount per |
|---|---|---|
| Component | % w/w | Capsule (mg) |
| Capsule, HPMC, Size 0, Orange | | 1 each |
| Opaque Body and Orange Opaque Cap | | |
| Titanium Dioxide | 1.5202 | 1.37-1.55 |
| Hypromellose | QSP 100 | QSP 90-102 |
| FD&C Yellow #6 | 0.5774 | 0.52-0.59 |

QSP = Quantité Suffisante Pour” (Quality Sufficient For)

Some embodiments provide a product or kit comprising a pharmaceutical composition as provided herein comprising entrectinib and one or more chemotherapeutic agents, as a combined preparation for simultaneous, separate or sequential use in anticancer therapy.

The present invention, thus generally described, will be understood more readily by reference to the following examples, which are provided by way of illustration and are not intended to be limiting of the present invention.

### EXAMPLES

### Example 1A

### Clinical Trial Experience (Interim Results and Analysis for N=355)

Entrectinib was studied in one dose-finding trial in adults [ALKA (n=57)], one dose-finding and activity-estimating trial in adults [STARTRK-1 (n=76)], one dose-finding and activity-estimating trial in pediatric and adult patients [STARTRK-NG (n=16)], and one single arm, activity-estimating trial in adults [STARTRK-2 (n=206)]. Of these 355 patients, 172 (48%) patients were exposed to entrectinib for 6 months or longer and 84 (24%) patients exposed to entrectinib for 1 year or longer.

The population characteristics were: median age 55 years (range: 4 to 86 years); 5% (n=17) were less than 18 years of age; 55% were female; and 66% were White, 23% were Asian, and 5% were Black; 3% were Hispanic/Latino. The most common tumors (≥5%) were lung (56%), sarcoma (8%), and colon (5%). ROS1 gene fusions were present in 42% and NTRK gene fusions were present in 20%. Most adults (75%) received entrectinib 600 mg orally once daily. The doses ranged from 100 mg/m² to 1600 mg/m² once daily in adults and 250 mg/m² to 750 mg/m² once daily in pediatric patients.

Severity of adverse reactions was graded as defined by National Cancer Institute Common Terminology Criteria for Adverse Events (NCI CTCAE) version 4.0.

Serious adverse reactions occurred in 39% of patients. The most frequent serious adverse reactions (≥2%) were pneumonia (3.9%), dyspnea (3.7%), pleural effusion (3.4%), sepsis (2.5%), pulmonary embolism (2.3%), respiratory failure (2%), and pyrexia (2%). Grade 3 or 4 adverse reactions occurred in 60% of patients; the most common (≥2%) were lung infection (5%), increased weight (7%), dyspnea (6%), fatigue/asthenia (5%), cognitive disorders (4.5%), syncope (2.5%), pulmonary embolism (3.4%), hypoxia (3.4%), pleural effusion (3.1%), hypotension (2.8%), diarrhea (2%), and urinary tract infection (2.5%). Fatal events included dyspnea (0.6%), pneumonia (0.6%), sepsis (0.6%), completed suicide (0.3%), large intestine perforation (0.3%) and tumor lysis syndrome (0.3%). One patient developed Grade 4 myocarditis after one dose of entrectinib which resolved after discontinuation of entrectinib and administration of high-dose corticosteroids.

Permanent discontinuation due to an adverse reaction occurred in 9% of patients who received entrectinib. The most frequent adverse reactions (<1% each) that resulted in permanent discontinuation were pneumonia, cardio-respiratory arrest, dyspnea, and fatigue.

Dose interruptions due to adverse reactions occurred in 46% of patients. The most frequent adverse reactions (≥2%) that resulted in interruption were increased blood creatinine (4%), fatigue (3.7%), anemia (3.1%), diarrhea (2.8%), pyrexia (2.8%), dizziness (2.5%), dyspnea (2.3%), nausea (2.3%), pneumonia (2.3%), cognitive disorder (2%) and neutropenia (2%).

Dose reductions due to adverse reactions occurred in 29% of patients who received entrectinib. The most frequent adverse reactions resulting in dose reductions (≥1%) were dizziness (3.9%), increased blood creatinine (3.1%), fatigue (2.3%), anemia (1.7%), and increased weight (1.4%).

The most common adverse reactions (≥20%) were fatigue, constipation, dysgeusia, edema, dizziness, diarrhea, nausea, dysesthesia, dyspnea, myalgia, cognitive impairment, increased weight, cough, vomiting, pyrexia, arthralgia and vision disorders.

Table 7 summarizes the adverse reactions observed in these 355 patients.

TABLE 7
Adverse Reactions (≥10%) in Patients Receiving Entrectinib
in ALKA, STARTRK-1, STARTRK-2, and STARTRK-NG

Entrectinib
N = 355

| | | |
|---|---|---|
| Adverse Reactions | All Grades (%) | Grade ≥3* (%) |

General

| | | |
|---|---|---|
| Fatigue¹ | 48 | 5 |
| Edema² | 40 | 1.1 |
| Pyrexia | 21 | 0.8 |

Gastrointestinal

| | | |
|---|---|---|
| Constipation | 46 | 0.6 |
| Diarrhea | 35 | 2.0 |
| Nausea | 34 | 0.3 |
| Vomiting | 24 | 0.8 |
| Abdominal pain³ | 16 | 0.6 |

Nervous System

| | | |
|---|---|---|
| Dysgeusia | 44 | 0.3 |
| Dizziness⁴ | 38 | 0.8 |
| Dysesthesia⁵ | 34 | 0.3 |
| Cognitive impairment⁶ | 27 | 4.5 |
| Peripheral sensory | 18 | 1.1 |
| neuropathy⁷ | | |
| Headache | 18 | 0.3 |
| Ataxia⁸ | 17 | 0.8 |
| Sleep⁹ | 14 | 0.6 |
| Mood disorders¹⁰ | 10 | 0.6 |

Respiratory, Thoracic and Mediastinal

| | | |
|---|---|---|
| Dyspnea | 30 | 6* |
| Cough | 24 | 0.3 |

Musculoskeletal and Connective Tissue

| | | |
|---|---|---|
| Myalgia¹¹ | 28 | 1.1 |
| Arthralgia | 21 | 0.6 |
| Muscular weakness | 12 | 0.8 |
| Back pain | 12 | 1 |
| Pain in extremity | 11 | 0.3 |

Metabolism and Nutritional

| | | |
|---|---|---|
| Increased weight | 25 | 7 |
| Decreased appetite | 13 | 0.3 |
| Dehydration | 10 | 1.1 |

Eye

| | | |
|---|---|---|
| Vision disorders¹² | 21 | 0.8 |

Infections

| | | |
|---|---|---|
| Urinary tract infection | 13 | 2.3 |
| Lung infection¹³ | 10 | 6* |

Vascular

| | | |
|---|---|---|
| Hypotension¹⁴ | 18 | 2.8 |

Skin and Subcutaneous Tissue

| | | |
|---|---|---|
| Rash¹⁵ | 11 | 0.8 |

*Grades 3-5, inclusive of fatal adverse reactions, including 2 events of pneumonia and 2 events of dyspnea.
¹Includes fatigue, asthenia
²Includes face edema, fluid retention, generalized edema, localized edema, edema, edema peripheral, peripheral swelling
³Includes abdominal pain upper, abdominal pain, lower abdominal discomfort, abdominal tenderness
⁴Includes dizziness, vertigo, dizziness postural
⁵Includes paresthesia, hyperesthesia, hypoesthesia, dysesthesia, oral hypoesthesia, palmar-plantar erythrodysesthesia, oral paresthesia, genital hypoesthesia
⁶Includes amnesia, aphasia, cognitive disorder, confusional state, delirium, disturbance in attention, hallucinations, visual hallucination, memory impairment, mental disorder, mental status changes
⁷Includes neuralgia, neuropathy peripheral, peripheral motor neuropathy, peripheral sensory neuropathy
⁸Includes ataxia, balance disorder, gait disturbances
⁹Includes hypersomnia, insomnia, sleep disorder, somnolence
¹⁰Includes anxiety, affect lability, affective disorder, agitation, depressed mood, euphoric mood, mood altered, mood swings, irritability, depression, persistent depressive disorder, psychomotor retardation
¹¹Includes musculoskeletal pain, musculoskeletal chest pain, myalgia, neck pain
¹²Includes blindness, cataract, cortical cataract, corneal erosion, diplopia, eye disorder, photophobia, photopsia, retinal hemorrhage, vision blurred, visual impairment, vitreous adhesions, vitreous detachment, vitreous floaters
¹³Includes lower respiratory tract infection, lung infection, pneumonia, respiratory tract infection
¹⁴Includes hypotension, orthostatic hypotension
¹⁵Includes rash, rash maculopapular, rash pruritic, rash erythematous, rash papular

Clinically relevant adverse reactions occurring in ≤10% of patients include dysphagia (10%), fall (8%), pleural effusion (8%), fractures (6%), hypoxia (4.2%), pulmonary embolism (3.9%), syncope (3.9%), congestive heart failure (3.4%), and QT prolongation (3.1%).

Table 8 summarizes the laboratory abnormalities.

TABLE 8
Laboratory Abnormalities (≥20%) Worsening
from Baseline in Patients Receiving Entrectinib
in ALKA, STARTRK-1, STARTRK-2, and STARTRK-NG

| | Entrectinib | |
|---|---|---|
| | NCI CTCAE Grade | |
| Laboratory Abnormality | All Grades (%)¹ | Grade 3 or 4 (%)¹ |

Hematology

| | | |
|---|---|---|
| Anemia | 67 | 9 |
| Lymphopenia | 40 | 12 |
| Neutropenia | 28 | 7 |

Chemistry

| | | |
|---|---|---|
| Increased creatinine² | 73 | 2.1 |
| Hyperuricemia | 52 | 10 |
| Increased AST | 44 | 2.7 |
| Increased ALT | 38 | 2.9 |
| Hypernatremia | 35 | 0.9 |
| Hypocalcemia | 34 | 1.8 |
| Hypophosphatemia | 30 | 7 |
| Increased lipase | 28 | 10 |
| Hypoalbuminemia | 28 | 2.9 |
| Increased amylase | 26 | 5.4 |
| Hyperkalemia | 25 | 1.5 |
| Increased alkaline phosphatase | 25 | 0.9 |
| Hyperglycemia³ | NE³ | 3.8 |

AST: Aspartate Aminotransferase;
ALT: Alanine Aminotransferase
¹ Denominator for each laboratory parameter is based on the number of patients with a baseline and post-treatment laboratory value available which ranged from 111 to 346 patients.
²Based on NCI CTCAE v5.0
³NE = Not evaluable. Grade 1 and 2 could not be determined per NCI CTCAE v5.0, as fasting glucose values were not collected.

The following clinically significant adverse reactions are described in more detail below: congestive heart failure, central nervous system effects, skeletal fractures, hepatotoxicity, hyperuricemia, QT interval prolongation, and vision disorders.

Congestive Heart Failure (CHF)

Among the 355 patients who received entrectinib across clinical trials, congestive heart failure (CHF) occurred in 3.4% of patients, including Grade 3 (2.3%). In clinical trials, baseline cardiac function and routine cardiac monitoring other than electrocardiograms (ECGs) were not conducted and eligibility criteria excluded patients with symptomatic CHF, myocardial infarction, unstable angina, and coronary artery bypass graft within 3 months of study entry. Among the 12 patients with CHF, the median time to onset was 2 months (range: 11 days to 12 months). entrectinib was interrupted in 6 of these patients (50%) and discontinued in 2 of these patients (17%). CHF resolved in 6 patients (50%) following interruption or discontinuation of entrectinib and institution of appropriate medical management. In addition, myocarditis in the absence of CHF was documented in 0.3% of patients.

Central Nervous System Effects

A broad spectrum of central nervous system (CNS) adverse reactions occurred in patients receiving entrectinib, including cognitive impairment, mood disorders, dizziness, and sleep disturbances.

Among the 355 patients who received entrectinib across clinical trials, 96 (27%) experienced cognitive impairment; symptoms occurred within 3 months of starting entrectinib in 74 (77%). Cognitive impairment included cognitive disorders (8%), confusional state (7%), disturbance in attention (4.8%), memory impairment (3.7%), amnesia (2.5%), aphasia (2.3%), mental status changes (2%), hallucinations (1.1%), and delirium (0.8%). Grade 3 cognitive adverse reactions occurred in 4.5% of patients. Among the 96 patients with cognitive impairment, 13% required a dose reduction, 18% required dose interruption and 1% discontinued entrectinib due to cognitive adverse reactions.

Among the 355 patients who received entrectinib across clinical trials, 36 (10%) experienced mood disorders. The median time to onset of mood disorders was 1 month (range: 1 day to 9 months). Mood disorders occurring in ≥1% of patients included anxiety (4.8%), depression (2.8%) and agitation (2%). Grade 3 mood disorders occurred in 0.6% of patients. One completed suicide was reported 11 days after treatment had ended. Among the 36 patients who experienced mood disorders, 6% required a dose reduction, 6% required dose interruption and no patients discontinued entrectinib due to mood disorders.

Dizziness occurred in 136 (38%) of the 355 patients. Among the 136 patients who experienced dizziness, Grade 3 dizziness occurred in 2.2% of patients. Ten percent of patients required a dose reduction, 7% required dose interruption and 0.7% discontinued entrectinib due to dizziness.

Among the 355 patients who received entrectinib across clinical trials, 51 (14%) experienced sleep disturbances. Sleep disturbances included insomnia (7%), somnolence (7%), hypersomnia (1.1%), and sleep disorder (0.3%). Grade 3 sleep disturbances occurred in 0.6% of patients. Among the 51 patients who experienced sleep disturbances, 6% required a dose reduction and no patients discontinued entrectinib due to sleep disturbances.

The incidence of CNS adverse reactions was similar in patients with and without CNS metastases; however, the incidence of dizziness (38% vs 31%), headache (21% vs 13%), paresthesia (20% vs 6%), balance disorder (13% vs 4%), and confusional state (11% vs 2%) appeared to be increased in patients with CNS metastases who had received prior CNS irradiation (N=90) compared to those who did not (N=48).

Skeletal Fractures

Entrectinib increases the risk of fractures. In an expanded safety population that included 338 adult patients and 30 pediatric patients who received entrectinib across clinical trials, 5% of adult patients and 23% of pediatric patients experienced fractures. In adult patients, some fractures occurred in the setting of a fall or other trauma to the affected area, while in pediatric patients, all fractures occurred in patients with minimal or no trauma. In general, there was inadequate assessment for tumor involvement at the site of fracture; however, radiologic abnormalities possibly indicative of tumor involvement were reported in some patients. In both adult and pediatric patients, most fractures were hip or other lower extremity fractures (e.g., femoral or tibial shaft). In a limited number of patients, bilateral femoral neck fractures occurred. The median time to fracture was 3.8 months (range 0.3 to 18.5 months) in adults and 4.0 months (range: 1.8 months to 7.4 months) in pediatric patients. Entrectinib was interrupted in 41% of adults and 43% of pediatric patients due to fractures. No patients discontinued entrectinib due to fractures.

Hepatotoxicity

Among the 355 patients who received entrectinib, increased AST of any grade occurred in 42% of patients and increased ALT of any grade occurred in 36%. Grade 3-4 increased AST or ALT occurred in 2.5% and 2.8% of patients, respectively; the incidence may be underestimated as 4.5% of patients had no post-treatment liver function tests. The median time to onset of increased AST was 2 weeks (range: 1 day to 29.5 months). The median time to onset of increased ALT was 2 weeks (range: 1 day to 9.2 months). Increased AST or ALT leading to dose interruptions or reductions occurred in 0.8% and 0.8% of patients, respectively. Entrectinib was discontinued due to increased AST or ALT in 0.8% patients.

Hyperuricemia

Among 355 patients who received entrectinib across clinical trials, 32 patients (9%) experienced hyperuricemia reported as adverse reactions with symptoms, as well as elevated uric acid levels. Grade 4 hyperuricemia occurred in 1.7% of patients, including one patient who died due to tumor lysis syndrome. Among the 32 patients with hyperuricemic adverse reactions, 34% required urate-lowering medication to reduce uric acid levels, 6% required dose reduction and 6% required dose interruption. Hyperuricemia resolved in 73% of patients following initiation of urate-lowering medication without interruption or dose reduction of entrectinib. No patients discontinued entrectinib due to hyperuricemia.

QT Interval Prolongation

Among the 355 patients who received entrectinib across the clinical trials, 3.1% of patients with at least one post-baseline ECG assessment experienced QTcF interval prolongation of >60 ms after starting entrectinib and 0.6% had a QTcF interval >500 ms.

Vision Disorders

Among the 355 patients who received entrectinib across clinical trials, vision changes occurred in 21% of patients, including Grade 1 (82%), Grade 2 (14%) and Grade 3 (0.8%). Vision disorders occurring in ≥1% included blurred vision (8.7%), photophobia (5.1%), diplopia (3.1%), visual impairment (2%), photopsia (1.3%), cataract (1.1%), and vitreous floaters (1.1%).

### Example 1B

### Clinical Trial Experience (Results and Analysis for N=504)

For the clinical development program of entrectinib, a total of 504 patients have received entrectinib in 4 clinical trials (ALKA, STARTRK-1, STARTRK-2 and STARTRK-NG). The safety of entrectinib was evaluated as integrated analyses of these 4 clinical trials. The median duration of exposure to entrectinib was 5.5 months.

The safety of entrectinib in adult patients has been evaluated in a total of 475 patients with NTRK-fusion positive, ROS1-positive or ALK-positive solid tumors, in studies ALKA, STARTRK-1, and STARTRK-2.

The safety of entrectinib in pediatric patients was established based on extrapolation of data from three open-label, single-arm clinical trials in adult patients with solid tumors harboring an NTRK gene fusion (ALKA, STARTRK-1 and STARTRK-2), and data from 32 pediatric patients (30 patients enrolled in STARTRK-NG, and 2 patients enrolled in STARTRK-2). Of these, 2 patients were less than 2 years old, 23 patients were 2 to 11 years old, 7 patients were 12 to 17 years old; 18 (56.3%) patients had metastatic disease, 14 (43.8%) patients had locally advanced disease; 27 (84.4%) patients had received prior treatment for their cancer, including surgery, radiotherapy, or systemic therapy. The most common cancers were neuroblastoma (N=13), inflammatory myofibroblastic tumors (N=3), glioblastoma (N=3), infantile sarcoma (N=2), ganglioneuroblastoma (N=2). The median duration of exposure for all pediatric patients was 5.57 months (range: 0.2 months to 29.8 months).

Adverse reactions and laboratory abnormalities of Grade 3 or 4 severity occurring more frequently (at least a 5% increased incidence) in pediatric patients compared to adult patients were neutropenia (28.1% vs. 3.4%), weight increased (21.9% vs 6.9%), headache (6.3% vs 0.6%) and bone fractures (12.5% vs 1.9%).

Among the 504 patients who received entrectinib across clinical trials, 130 (25.8%) patients were 65 years or older and 34 (6.7%) were 75 years or older. The overall safety profile of entrectinib in the elderly patients is similar to the safety profile observed in patients younger than 65 years of age. Adverse reactions occurring more frequently in the elderly compared to patients less than 65 years old were dizziness (48.5% vs 36.6%), blood creatinine increased (31.5% vs 23.3%), and hypotension (21.5% vs 14.7%), ataxia (23.8% vs 12.8%).

Table 9 summarizes the adverse reactions observed in these 504 patients. Adverse drug reactions from the clinical trials are listed by MedDRA system organ class. The following categories of frequency have been used: very common ≥1/10, common (≥1/100 to <1/10), uncommon (≥1/1,000 to <1/100), rare (≥1/10,000 to <1/1000), very rare (<1/10,000).

TABLE 9
Adverse Reactions (≥10%) in Patients Receiving Entrectinib
in ALKA, STARTRK-1, STARTRK-2, and STARTRK-NG

| | Entrectinib | | Frequency |
|---|---|---|---|
| System Organ Class | N = 504 | | Category |
| Adverse Reaction | All Grades (%) | Grade ≥3 (%) | (All Grades) |

General Disorders and Administration Site Conditions

| | | | |
|---|---|---|---|
| Fatigue¹⁴ | 45.0 | 5.0 | very common |
| Edema⁶ | 37.3 | 1.4 | very common |
| Pain⁷ | 24.4 | 1.6 | very common |
| Pyrexia | 20.0 | 0.8 | very common |

Gastrointestinal Disorders

| | | | |
|---|---|---|---|
| Constipation | 42.9 | 0.4 | very common |
| Diarrhea | 33.5 | 2.6 | very common |
| Nausea | 32.1 | 0.8 | very common |
| Vomiting | 23.2 | 1.2 | very common |
| Abdominal pain | 11.1 | 0.6 | very common |
| Dysphagia | 10.1 | 0.4 | very common |

Nervous System Disorders

| | | | |
|---|---|---|---|
| Dysgeusia | 42.3 | 0.4 | very common |
| Dizziness⁵ | 39.7 | 1.2 | very common |
| Dysasthesia³ | 29.0 | 0.2 | very common |
| Cognitive Disorders¹ | 24.2 | 4.4 | very common |
| Headache | 17.5 | 1.0 | very common |
| Peripheral Sensory | 15.7 | 1.0 | very common |
| Neuropathy² | | | |
| Ataxia⁴ | 15.7 | 0.8 | very common |
| Sleep disturbances¹⁶ | 13.5 | 0.4 | very common |
| Mood disorders¹⁷ | 9.1 | 0.6 | common |
| Syncope | 4.6 | 3.0 | common |

Respiratory Disorders

| | | | |
|---|---|---|---|
| Dyspnea | 27.0 | 5.8* | very common |
| Cough | 21.4 | 0.6 | very common |

Blood Disorders

| | | | |
|---|---|---|---|
| Anemia | 28.2 | 9.7 | very common |
| Neutropenia¹⁰ | 11.3 | 4.4 | very common |

Metabolism and Nutritional Disorders

| | | | |
|---|---|---|---|
| Weight increased | 26.4 | 7.3 | very common |
| Decreased appetite | 11.9 | 0.2 | very common |
| Hyperuricemia | 9.1 | 1.8 | common |
| Dehydration | 7.9 | 1.0 | common |
| Tumor lysis syndrome | 0.2 | 0.2* | uncommon |

Renal and urinary disorders

| | | | |
|---|---|---|---|
| Blood creatinine | 25.4 | 0.6 | very common |
| increased | | | |

Musculoskeletal Disorders

| | | | |
|---|---|---|---|
| Myalgia | 19.6 | 0.6 | very common |
| Arthralgia | 19.0 | 0.6 | very common |
| Muscular weakness | 12.3 | 1.2 | very common |
| Fractures¹¹ | 6.2 | 2.4 | common |

Hepatobiliary Disorders

| | | | |
|---|---|---|---|
| AST increased | 17.5 | 3.6 | very common |
| ALT increased | 16.1 | 3.4 | very common |

Infections and Infestations

| | | | |
|---|---|---|---|
| Lung infection⁸ | 13.1 | 6.0* | very common |
| Urinary tract infection | 12.7 | 2.6 | very common |

Eye Disorders

| | | | |
|---|---|---|---|
| Vision Blurred¹³ | 11.9 | 0.4 | very common |

Skin and Subcutaneous Tissue Disorders

| | | | |
|---|---|---|---|
| Rash¹² | 11.5 | 1.4 | very common |

Vascular Disorders

| | | | |
|---|---|---|---|
| Hypotension¹⁵ | 16.5 | 2.4 | very common |

Cardiac Disorders

| | | | |
|---|---|---|---|
| Congestive Heart | 3.0 | 2.2 | common |
| Failure⁹ | | | |
| Electrocardiogram | 2.0 | 0.6 | common |
| QT prolonged | | | |

ALT: Alanine aminotransferase
AST: Aspartate aminotransferase
*Grades 3 to 5, inclusive of fatal adverse reactions (including 2 reactions of pneumonia, 2 reactions of dyspnea, and 1 reaction of tumor lysis syndrome)
¹Includes preferred terms: cognitive disorder, confusional state, disturbance in attention, memory impairment, amnesia, mental status changes, hallucination, delirium, ‘hallucination visual’ and mental disorder.
²Includes the preferred terms: neuralgia, neuropathy peripheral, peripheral motor neuropathy, peripheral sensory neuropathy
³Includes the preferred terms: paresthesia, hyperesthesia, hypoesthesia, dysesthesia
⁴Includes the preferred terms: ataxia, balance disorder, gait disturbances
⁵Includes the preferred terms: dizziness, vertigo, dizziness postural
⁶Includes the preferred terms: face edema, fluid retention, generalized edema, localized edema, edema, edema peripheral, peripheral swelling
⁷Includes the preferred terms: back pain, neck pain, musculoskeletal chest pain, musculoskeletal pain, pain in extremity
⁸Includes the preferred terms: bronchitis, lower respiratory tract infection, lung infection, pneumonia, respiratory tract infection, upper respiratory tract infection
⁹Includes the preferred terms: acute right ventricular failure, cardiac failure, cardiac failure congestive, chronic right ventricular failure, ejection fraction decreased, pulmonary edema
¹⁰Includes the preferred terms: neutropenia, neutrophil count decreased
¹¹Includes the preferred terms: humerus fracture, foot fracture, ankle fracture, femoral neck fracture, stress fracture, fibula fracture, fracture, rib fracture, spinal fracture, wrist fracture, femur fracture, pathological fracture
¹²Includes the preferred terms: rash, rash maculopapular, rash pruritic, rash erythematous, rash papular
¹³Includes the preferred terms: diplopia, vision blurred, visual impairment
¹⁴Includes the preferred terms: fatigue, asthenia
¹⁵Includes the preferred terms: hypotension, orthostatic hypotension
¹⁶Includes the preferred terms: hypersomnia, insomnia, sleep disorder, somnolence
¹⁷Includes the preferred terms: anxiety, affect lability, affective disorder, agitation, depressed mood, euphoric mood, mood altered, mood swings, irritability, depression, persistent depressive disorder, psychomotor retardation

Table 10 summarizes the laboratory abnormalities.

TABLE 10
Laboratory Abnormalities (≥20%) Worsening
from Baseline in Patients Receiving Entrectinib
in ALKA, STARTRK-1, STARTRK-2, and STARTRK-NG

| | Entrectinib | |
|---|---|---|
| | NCI-CTCAE Grade | |
| | N = 504² | |
| Laboratory Test | Change from Baseline | Change from Baseline |
| Abnormality¹ | All Grades (%) | to Grade 3 or 4 (%)³ |
| Chemistry | | |
| Increased Blood | 94.8 | 3.1 |
| Creatinine | | |
| Hyperuricemia | 50.8 | 6.8 |
| Increased AST | 43.3 | 3.3 |
| Increased ALT | 38.4 | 3.1 |
| Hematology | | |
| Decreased Neutrophils | 27.8 | 6.3 |
| Decreased Hemoglobin | 65.7 | 9.2 |

AST: Aspartate Aminotransferase;
ALT: Alanine Aminotransferase
¹Based on number of patients with available baseline and at least one on-treatment test value
²N = 480 for Blood Creatinine; N = 478 for AST; N = 479 for ALT; N = 382 for Hyperuricemia; N = 457 for Neutrophils; N = 487 for Hemoglobin
³Patients with change from baseline values of Grade of 0-2 to a post-baseline value of Grade 3 or Grade 4 at any time

Select adverse drug reactions are discussed in further detail below.

Cognitive Disorders

A variety of cognitive symptoms were reported across the clinical trials. These included events reported as cognitive disorders (6.3%), confusional state (7.3%), disturbance in attention (3.8%), memory impairment (4.2%), amnesia (2.8%), mental status changes (1.2%), hallucination (1.0%), delirium (0.8%), hallucination visual (0.4%) and mental disorder (0.2%). Grade 3 events were reported in 4.4% of patients. In the pediatric population, 3.4% (1/29) pediatric patients experienced disturbance in attention of Grade 1 severity. Patients who had brain metastases at baseline had a higher frequency of these events (29.7%) compared to those without brain metastases (23.1%).

Fractures

Fractures were experienced by 5.3% (N=475) of adult patients and 20.7% (N=29) of pediatric patients. In general, there was inadequate assessment for tumor involvement at the site of fracture; however, radiologic abnormalities possibly indicative of tumor involvement were reported in some patients. In both adult and pediatric patients, most fractures were hip or other lower extremity fractures (e.g., femoral or tibial shaft). In 2 pediatric patients, bilateral femoral neck fractures occurred. No patients discontinued entrectinib due to fractures.

In adult patients, some fractures occurred in the setting of a fall or other trauma to the affected area. The median time to fracture was 3.42 months (range: 0.26 months to 18.5 months) in adults. Entrectinib was interrupted due to fractures in 36.0% of adult patients.

In pediatric patients, all fractures occurred in patients with minimal or no trauma. The median time to fracture was 3.38 months (range: 1.77 months to 7.39 months) in pediatric patients. Entrectinib was interrupted due to fractures in 33.3% of pediatric patients.

Ataxia

Ataxia (including events of ataxia, balance disorder, and gait disturbances) was reported in 15.7% of patients. The median time to onset for ataxia was 0.36 months (range: 0.03 months to 28.19 months) and the median duration was 0.66 months (range: 0.03 months to 11.99 months). The majority of patients (67.1%) recovered from ataxia. Ataxia related adverse events were observed more frequently in elderly patients (23.8%) compared to patients below 65 years of age (12.8%).

Syncope

Syncope events were reported in 4.6% of patients. In some patients, syncope was reported with concurrent hypotension, dehydration, or QTc prolongation and in other patients no other concurrent related conditions were reported.

QTc Interval Prolongation

Among the 504 patients who received entrectinib across clinical trials, 17 (4.0%) patients with at least one post-baseline ECG assessment experienced QTcF interval prolongation of >60 ms after starting entrectinib, and 12 (2.8%) patients had a QTcF interval of ≥500 ms.

Peripheral Sensory Neuropathy

Peripheral sensory neuropathy was reported in 15.7% of patients. The median time to onset was 0.49 months (range 0.03 months to 20.93 months) and the median duration was 0.76 months (range: 0.07 months to 6.01 months). The majority of patients (55.7%) recovered from peripheral neuropathy.

Eye Disorders

Eye disorders reported across clinical trials included events of vision blurred (8.5%), diplopia (2.6%), and visual impairment (1.6%). The median time to onset for eye disorders was 1.87 months (range: 0.03 months to 21.59 months). The median duration of eye disorders was 1.02 months (range 0.03 months to 14.49 months). The majority of patients (61.7%) recovered from the eye disorder events.

### Example 2

### Clinical Trial in Patients with Impaired Hepatic Function

This is a non-randomized, open-label, one treatment, four group, parallel group study to investigate the effect of impaired hepatic function on the pharmacokinetics of entrectinib in volunteers with different levels of hepatic function. Volunteers with mild, moderate or severe hepatic impairment (‘Mild’, ‘Moderate’ and ‘Severe’ groups), and control subjects with normal hepatic function (‘Normal’ group) each receives a single 100 mg dose of entrectinib after consumption of a standardized meal.

The primary objectives are (1) to investigate the effect of impaired hepatic function on the pharmacokinetics of entrectinib and (2) to explore the relationship between entrectinib pharmacokinetic parameters and measures of hepatic function. A secondary objective is to explore the safety and tolerability of a single 100 mg oral dose of entrectinib in groups of volunteers with reduced hepatic function and a control group of volunteers with normal hepatic function.

The primary endpoints are
(1) the calculation of the geometric mean ratio and associated 90% confidence intervals of total and unbound entrectinib and M5 AUC_{inf} and Cₘₐₓ parameters between groups of volunteers with reduced hepatic function and a control group of volunteers with normal hepatic function. Duplicate analyses will be performed for groups based on Child-Pugh and NCI-ODWG classifications.
(2) use of linear or nonlinear models or regression analyses of the relationship between entrectinib pharmacokinetic parameters (e.g. Cmax and AUCinf) and selected hepatic function parameters (e.g., Child-Pugh scores, albumin, bilirubin, or aspartate aminotransferase [AST] concentrations, prothrombin time)

A secondary endpoint is incidence and severity of adverse events, incidence of abnormalities in laboratory safety tests, 12-lead ECGs, and vital sign measurements.

Volunteers with reduced hepatic function are assigned to a functional category based on assessments at the Screening visit. Each individual is categorized according to the Child Pugh system for classifying hepatic impairment and also according to the National Cancer Institute (NCI) organ dysfunction working group (NCI-ODWG) system.

The control group of volunteers with normal hepatic function are matched to the Mild, Moderate and Severe groups so that key demographic characteristics of the control group are comparable with those of the overall population of volunteers with hepatic impairment, i.e. ranges of weight and age, and numbers of volunteers of each sex, are similar.

The study treatments are administered orally as 1×100 mg capsule, and given with approximately 240 mL of water within 30 minutes of consumption of a standardized meal.

Blood samples for measurement of plasma concentrations entrectinib and its active metabolite M5 are collected before and at intervals up to 144 hr after study drug dosing. In addition, samples are taken for ex vivo measurement of plasma protein binding. Safety and tolerability are monitored by clinical and laboratory assessments at intervals throughout the study.

The total duration of the study for each enrolled subject (screening through to end of study) is up to 8 weeks, divided as follows:
Screening: up to 28 days before the first dose of study drug
One treatment period
Safety follow-up: 12 to 14 days after study drug administration

Volunteers who fulfill the following inclusion criteria are eligible for participation in the study:

### All Volunteers

1. Male or female
2. Aged 18 to 75 years of age, inclusive, at screening.
3. A body mass index (BMI) between 18.0 and 35.0 kg/m², and weighing at least 50 kg, at screening.
4. Agreement to comply with measures to prevent pregnancy and restrictions on sperm donation.
5. Able and willing to give written informed consent and to comply with study protocol and study restrictions.
   Volunteers with Normal Hepatic Function
6. Normal hepatic function and no history of clinically significant hepatic dysfunction.
7. Healthy in the opinion of the Investigator. Healthy is defined by the absence of evidence of any active disease or clinically significant medical condition based on a detailed medical history, physical examination, vital signs and 12-lead ECG assessment, and laboratory safety test results.
   Volunteers with Hepatic Impairment
8. Mild, moderate or severe hepatic dysfunction (i.e. Child-Pugh A, B or C) arising from cirrhosis of the liver as the result of parenchymal liver disease. Cirrhosis should be documented by medical history including confirmation of the diagnosis by liver biopsy, hepatic ultrasound, computerized tomography (CT) scan or magnetic resonance imaging (MRI) scan.
9. Stable hepatic function and receiving a stable treatment regimen. Stable hepatic function is defined as no clinically significant change in disease status within the 30 days preceding screening, as documented by the subject's recent medical history (e.g., no worsening of clinical signs of hepatic impairment, or no worsening of total bilirubin or prothrombin by more than 50%). Stable treatment is defined as no alteration to relevant concomitant medication treatment regimen, according to the Investigator's clinical judgment, within the 30 days preceding screening.

Participants are not eligible for the study if any of the following criteria are met:

### All Volunteers

1. Life expectancy<1 year.
2. Transjugular intrahepatic portosystemic shunt or other porta-caval shunt.
3. A history of gastrointestinal hemorrhage due to esophageal varices or peptic ulcers within the three months preceding screening.
4. Recent history (i.e. within the last 6 months prior to screening) or signs of severe hepatic encephalopathy (e.g., a portal systemic encephalopathy score >2).
5. Severe ascites or pleural effusion.
6. Hepatocellular carcinoma, acute liver disease (e.g., caused by an infection or drug toxicity) or serum ALT or AST>5×upper limit of normal at screening.
7. Recipient of a liver transplant.
8. Uncontrolled hypertension, defined as SBP>160 mmHg and/or DBP≥105 mmHg.
9. Clinically significant impairment of renal function, defined as a creatinine clearance of <60 mL/min estimated from serum creatinine concentrations using an appropriate method.
10. A history of gastrointestinal surgery (e.g. gastric bypass) or other gastrointestinal disorder (e.g. malabsorption syndrome) that might affect absorption of medicines from the gastrointestinal tract. Cholecystectomy is permissible.
11. Clinically significant change in health status, as judged by the Investigator, or any major illness within the four weeks before screening, or clinically significant acute infection or febrile illness within the 14 days before screening.
12. Apart from hepatic dysfunction, any other ongoing condition or disease, or laboratory test result, that the investigator considers would render the participant unsuitable for the study, place the subject at undue risk, interfere with the ability of the subject to complete the study, or confound interpretation of study data. Concurrent chronic disorders such as hypertension and diabetes are permissible providing they are stable and adequately controlled.
13. Women who are pregnant or lactating.
14. QTcF interval >470 msec or the presence of any other abnormal ECG finding, which, in the Investigator's opinion, is clinically significant.
15. Use of moderate or potent inhibitors or inducers of cytochrome P450 3A4 enzyme or P-glycoprotein transporter within the 28 days before screening, or use of other prohibited medications within the 7 days before screening.
16. Participation in any other clinical study involving an investigational medicinal product or device within 3 months before screening.
17. A positive test result for human immunodeficiency virus (HIV).
18. Recent history (i.e. within the last 6 months prior to screening) of alcoholism, drug abuse, or drug addiction, or a positive test for alcohol or drugs of abuse. Alcohol consumption not exceeding 2 standard drinks per day on average (i.e. average daily alcohol consumption less than approximately 20 g of alcohol) is permitted, although volunteers should not consume alcohol in the 48 hrs preceding study center visits or during the period of in-patient confinement.
19. Known history of clinically significant hypersensitivity, or severe allergic reaction, to entrectinib or related compounds or other excipients in the entrectinib formulation.
20. Donation or loss of over 500 mL of blood within the three months before screening.

Plasma concentrations of entrectinib and its active metabolite M5, and derived plasma pharmacokinetic parameters, are listed and summarized by group using descriptive statistics. Individual and mean concentration versus time profiles are plotted. Separate summaries are presented for groups categorized according to the Child Pugh and NCI-ODWG system for classifying hepatic function. In effect, there may be eight analysis groups (i.e. Normal Child-Pugh, Normal NCI-ODWG, Mild Child-Pugh, Mild NCI-ODWG, Moderate Child-Pugh, Moderate NCI-ODWG, Severe Child-Pugh, and Severe NCI-ODWG).

The following pharmacokinetic parameters are derived using standard non-compartmental methods:
Cₘₐₓ: Maximum observed plasma concentration
AUC_{inf}: Area under the plasma entrectinib concentration-time curve from time 0 extrapolated to infinity.
Tₘₐₓ: Time of Maximum observed plasma concentration
t_{1/2}: Apparent terminal elimination half-life
M:P ratio: Molecular weight-adjusted metabolite to parent ratio based on AUC_{inf}
CL/F: Apparent oral clearance (entrectinib only)
V/F: Apparent volume of distribution (entrectinib only)
fu: Fraction of drug unbound in plasma

The fractions of entrectinib and M5 unbound in plasma (fu) are determined by ex vivo protein binding assay and used to calculate exposure parameters adjusted for protein binding (i.e., unbound Cₘₐₓ, unbound AUC_{inf}).

An analysis of variance (ANOVA) is used to estimate the effect of hepatic impairment on log-transformed primary pharmacokinetic parameters and will include the factor hepatic impairment (i.e. mild, moderate and severe, and none). Estimates of geometric mean ratios on the original scale, together with the corresponding 90% confidence intervals (CIs), are derived for the comparisons between each hepatic impairment group and the control group with normal hepatic function. Duplicate analyses are performed for groups based on Child-Pugh and NCI-ODWG classifications.

The relationship between entrectinib pharmacokinetic parameters (e.g. Cₘₐₓ and AUC_{inf}) and measures of hepatic function (e.g., Child-Pugh scores, albumin, bilirubin, or AST concentrations, prothrombin time etc.) are explored using linear or non-linear models or regression analyses, as appropriate.

Para. A. A method of treating cancer in a patient in need thereof, the method comprising administering to the patient a therapeutically effective amount of entrectinib wherein the patient has impaired hepatic function.

Para. B. A method of treating cancer in a patient in need thereof, the method comprising administering to the patient a therapeutically effective amount of entrectinib wherein the patient has been identified as having impaired hepatic function prior to treatment.

Para. C. The method of Para. A or Para. B, wherein the impaired hepatic function is mild hepatic dysfunction, as assessed by Child-Pugh scoring system.

Para. D. The method of Para. A or Para. B, wherein the impaired hepatic function is moderate hepatic dysfunction, as assessed by Child-Pugh scoring system.

Para. E. The method of Para. A or Para. B, wherein the impaired hepatic function is severe hepatic dysfunction, as assessed by Child-Pugh scoring system.

Para. F. The method of any one of Paras. C-E, wherein the Child-Pugh scoring system comprises assessment of encephalopathy grade, ascites, serum bilirubin concentration, serum albumin concentration, and prothrombin time.

Para. G. The method of any one of Paras. C-F, wherein the mild, moderate, or severe hepatic dysfunction arises from liver cirrhosis.

Para. H. The method of Para. G, wherein the liver cirrhosis is a result of parenchymal liver disease.

Para. I. The method of Para. A or Para. B, wherein the impaired hepatic function is mild, moderate, or severe hepatic impairment as assessed by National Cancer Institute organ dysfunction working group system.

Para. J. The method of Para. I, wherein the National Cancer Institute organ dysfunction working group system measures total bilirubin concentration and aspartate aminotransferase concentration.

Para. K. The method of Para. A or Para. B, wherein the patient has a total bilirubin concentration that is about greater than 1.5 times the upper limit of normal to about 3 times the upper limit of normal.

Para. L. The method of Para. A or Para. B, wherein the patient has a total bilirubin concentration that is about 1.6 times the upper limit of normal to about 3 times the upper limit of normal.

Para. M. The method of any one of Paras. A-L, wherein the therapeutically effective amount is 600 mg or less.

Para. N. The method of any one of Paras. A-M, wherein the therapeutically effective amount is 100 mg, 200 mg, 300 mg, 400 mg, or 500 mg.

Para. O. The method of any one of Paras. A-N, wherein entrectinib is administered once daily.

Para. P. The method of any one of Paras. A-N, wherein entrectinib is administered twice daily.

Para. Q. The method of any one of Paras. A-P, wherein the patient has at least one genetic alteration in at least one target gene selected from ROS1, NTRK1, NTRK2, and NTRK3.

Para. R. The method of any one of Paras. A-P, wherein the patient has a genetic alteration in ROS1.

Para. S. The method of any one of Paras. A-P, wherein the patient has a genetic alteration in NTRK1, NTRK2, NTRK3, or any combination thereof.

Para. T. The method of any one of Paras. A-S, wherein the cancer is selected from sarcoma, non-small cell lung cancer, mammary analogue secretory carcinoma, breast cancer, thyroid cancer, colorectal cancer, neuroendocrine cancers, pancreatic cancer, gynecological cancers, and cholangiocarcinoma.

Para. U. The method of any one of Paras. A-P, wherein the cancer comprises one or more NTRK gene fusion-positive solid tumors.

Para. V. The method of any one of Paras. A-P, wherein the cancer is ROS-1 positive non-small cell lung cancer.

Para. AA. Entrectinib for use in the treatment of cancer in a patient, wherein the patient has impaired hepatic function.

Para. AB. Entrectinib for use in the treatment of cancer in a patient, wherein the patient has been identified as having impaired hepatic function.

Para. AC. Entrectinib for use in the treatment of cancer in a patient, wherein the patient has been identified as having impaired hepatic function prior to treatment.

Para. AD. Entrectinib for use in the treatment of cancer in a patient who has impaired hepatic function.

Para. AE. Entrectinib for use in the treatment of cancer in a patient who has been identified as having impaired hepatic function.

Para. AF. Entrectinib for use in the treatment of cancer in a patient who has been identified as having impaired hepatic function prior to treatment.

Para. AG. Entrectinib for use in the treatment of cancer in a subject being treated who has impaired hepatic function.

Para. AH. Entrectinib for use in the treatment of cancer in a subject being treated who has been identified as having impaired hepatic function.

Para. AI. Entrectinib for use in the treatment of cancer in a subject being treated who has been identified as having impaired hepatic function prior to treatment.

Para. AJ. Entrectinib for use in the treatment of cancer in a patient, wherein the patient treated has impaired hepatic function.

Para. AK. Entrectinib for use in the treatment of cancer in a patient, wherein the patient treated has been identified as having impaired hepatic function.

Para. AL. Entrectinib for use in the treatment of cancer in a patient, wherein the patient treated has been identified as having impaired hepatic function prior to treatment.

Para. AM. Entrectinib for use in the treatment of cancer in a patient, wherein impaired hepatic function has been determined in the patient.

Para. AN. Entrectinib for use in the treatment of cancer in a patient, wherein impaired hepatic function has been determined in the patient prior to treatment.

Para. AO. Entrectinib for use in the treatment according to any one of Paras. AA-AN, wherein the impaired hepatic function is mild hepatic dysfunction, as assessed by Child-Pugh scoring system.

Para. AP. Entrectinib for use in the treatment according to any one of Paras. AA-AN, wherein the impaired hepatic function is moderate hepatic dysfunction, as assessed by Child-Pugh scoring system.

Para. AQ. Entrectinib for use in the treatment according to any one of Paras. AA-AN, wherein the impaired hepatic function is severe hepatic dysfunction, as assessed by Child-Pugh scoring system.

Para. AR. Entrectinib for use in the treatment according to any one of Paras. AO-AQ, wherein the Child-Pugh scoring system comprises assessment of encephalopathy grade, ascites, serum bilirubin concentration, serum albumin concentration, and prothrombin time.

Para. AS. Entrectinib for use in the treatment according to any one of Paras. AO-AR, wherein the mild, moderate, or severe hepatic dysfunction arises from liver cirrhosis.

Para. AT. Entrectinib for use in the treatment according to Para. AS, wherein the liver cirrhosis is a result of parenchymal liver disease.

Para. AU. Entrectinib for use in the treatment according to any one of Paras. AA-AN, wherein the impaired hepatic function is mild, moderate, or severe hepatic impairment as assessed by National Cancer Institute organ dysfunction working group system.

Para. AV. Entrectinib for use in the treatment according to Para. AU, wherein the National Cancer Institute organ dysfunction working group system measures total bilirubin concentration and aspartate aminotransferase concentration.

Para. AW. Entrectinib for use in the treatment according to any one of Paras. AA-AN, wherein the patient has a total bilirubin concentration that is about greater than 1.5 times the upper limit of normal to about 3 times the upper limit of normal.

Para. AX. Entrectinib for use in the treatment according to any one of Paras. AA-AN, wherein the patient has a total bilirubin concentration that is about 1.6 times the upper limit of normal to about 3 times the upper limit of normal.

Para. AY. Entrectinib for use in the treatment according to any one of Paras. AA-AX, wherein the therapeutically effective amount is 600 mg or less.

Para. AZ. Entrectinib for use in the treatment according to any one of Paras. AA-AY, wherein the therapeutically effective amount is 100 mg, 200 mg, 300 mg, 400 mg, or 500 mg.

Para. BA. Entrectinib for use in the treatment according to any one of Paras. AA-AZ, wherein entrectinib is administered once daily.

Para. BB. Entrectinib for use in the treatment according to any one of Paras. AA-AZ, wherein entrectinib is administered twice daily.

Para. BC. Entrectinib for use in the treatment according to any one of Paras. AA-BB, wherein the patient has at least one genetic alteration in at least one target gene selected from ROS l, NTRK1, NTRK2, and NTRK3.

Para. BD. Entrectinib for use in the treatment according to any one of Paras. AA-BB, wherein the patient has a genetic alteration in ROS1.

Para. BE. Entrectinib for use in the treatment according to any one of Paras. AA-BB, wherein the patient has a genetic alteration in NTRK1, NTRK2, NTRK3, or any combination thereof.

Para. BF. Entrectinib for use in the treatment according to any one of Paras. AA-BE, wherein the cancer is selected from sarcoma, non-small cell lung cancer, mammary analogue secretory carcinoma, breast cancer, thyroid cancer, colorectal cancer, neuroendocrine cancers, pancreatic cancer, gynecological cancers, and cholangiocarcinoma.

Para. BG. Entrectinib for use in the treatment according to any one of Paras. AA-BB, wherein the cancer comprises one or more NTRK gene fusion-positive solid tumors.

Para. BH. Entrectinib for use in the treatment according to any one of Paras. AA-BB, wherein the cancer is ROS-1 positive non-small cell lung cancer.

Para. CA. Use of entrectinib in the manufacture of a medicament for the treatment of cancer in a patient, wherein the patient has impaired hepatic function.

Para. CB. Use of entrectinib in the manufacture of a medicament for the treatment of cancer in a patient, wherein the patient has been identified as having impaired hepatic function.

Para. CC. Use of entrectinib in the manufacture of a medicament for the treatment of cancer in a patient, wherein the patient has been identified as having impaired hepatic function prior to treatment.

Para. CD. Use of entrectinib in the manufacture of a medicament for the treatment of cancer in a patient who has impaired hepatic function.

Para. CE. Use of entrectinib in the manufacture of a medicament for the treatment of cancer in a patient who has been identified as having impaired hepatic function.

Para. CF. Use of entrectinib in the manufacture of a medicament for the treatment of cancer in a patient who has been identified as having impaired hepatic function prior to treatment.

Para. CG. Use of entrectinib in the manufacture of a medicament for the treatment of cancer in a subject being treated who has impaired hepatic function.

Para. CH. Use of entrectinib in the manufacture of a medicament for the treatment of cancer in a subject being treated who has been identified as having impaired hepatic function.

Para. CI. Use of entrectinib in the manufacture of a medicament for the treatment of cancer in a subject being treated who has been identified as having impaired hepatic function prior to treatment.

Para. CJ. Use of entrectinib in the manufacture of a medicament for the treatment of cancer in a patient, wherein the patient treated has impaired hepatic function.

Para. CK. Use of entrectinib in the manufacture of a medicament for the treatment of cancer in a patient, wherein the patient treated has been identified as having impaired hepatic function.

Para. CL. Use of entrectinib in the manufacture of a medicament for the treatment of cancer in a patient, wherein the patient treated has been identified as having impaired hepatic function prior to treatment.

Para. CM. Use of entrectinib in the manufacture of a medicament for the treatment of cancer in a patient, wherein impaired hepatic function has been determined in the patient.

Para. CN. Use of entrectinib in the manufacture of a medicament for the treatment of cancer in a patient, wherein impaired hepatic function has been determined in the patient prior to treatment.

Para. CO. The use according to any one of Paras. CA-CN, wherein the impaired hepatic function is mild hepatic dysfunction, as assessed by Child-Pugh scoring system.

Para. CP. The use according to any one of Paras. CA-CN, wherein the impaired hepatic function is moderate hepatic dysfunction, as assessed by Child-Pugh scoring system.

Para. CQ. The use according to any one of Paras. CA-CN, wherein the impaired hepatic function is severe hepatic dysfunction, as assessed by Child-Pugh scoring system.

Para. CR. The use according to any one of Paras. CO-CQ, wherein the Child-Pugh scoring system comprises assessment of encephalopathy grade, ascites, serum bilirubin concentration, serum albumin concentration, and prothrombin time.

Para. CS. The use according to any one of Paras. CO-CR, wherein the mild, moderate, or severe hepatic dysfunction arises from liver cirrhosis.

Para. CT. The use according to Para. CS, wherein the liver cirrhosis is a result of parenchymal liver disease.

Para. CU. The use according to any one of Paras. CA-CN, wherein the impaired hepatic function is mild, moderate, or severe hepatic impairment as assessed by National Cancer Institute organ dysfunction working group system.

Para. CV. The use according to Para. CU, wherein the National Cancer Institute organ dysfunction working group system measures total bilirubin concentration and aspartate aminotransferase concentration.

Para. CW. The use according to any one of Paras. CA-CN, wherein the patient has a total bilirubin concentration that is about greater than 1.5 times the upper limit of normal to about 3 times the upper limit of normal.

Para. CX. The use according to any one of Paras. CA-CN, wherein the patient has a total bilirubin concentration that is about 1.6 times the upper limit of normal to about 3 times the upper limit of normal.

Para. CY. The use according to any one of Paras. CA-CX, wherein the therapeutically effective amount is 600 mg or less.

Para. CZ. The use according to any one of Paras. CA-CY, wherein the therapeutically effective amount is 100 mg, 200 mg, 300 mg, 400 mg, or 500 mg.

Para. DA. The use according to any one of Paras. CA-CZ, wherein entrectinib is administered once daily.

Para. DB. The use according to any one of Paras. CA-CZ, wherein entrectinib is administered twice daily.

Para. DC. The use according to any one of Paras. CA-DB, wherein the patient has at least one genetic alteration in at least one target gene selected from ROS1, NTRK1, NTRK2, and NTRK3.

Para. DD. The use according to any one of Paras. CA-DB, wherein the patient has a genetic alteration in ROS1.

Para. DE. The use according to any one of Paras. CA-DB, wherein the patient has a genetic alteration in NTRK1, NTRK2, NTRK3, or any combination thereof.

Para. DF. The use according to any one of Paras. CA-DE, wherein the cancer is selected from sarcoma, non-small cell lung cancer, mammary analogue secretory carcinoma, breast cancer, thyroid cancer, colorectal cancer, neuroendocrine cancers, pancreatic cancer, gynecological cancers, and cholangiocarcinoma.

Para. DG. The use according to any one of Paras. CA-DB, wherein the cancer comprises one or more NTRK gene fusion-positive solid tumors.

Para. DH. The use according to any one of Paras. CA-DB, wherein the cancer is ROS-1 positive non-small cell lung cancer.

Para. EA. A method of treating cancer in a patient in need thereof, the method comprising:
initiating administration of entrectinib to the patient with an initial dose;
monitoring the patient for adverse reaction; and
continuing to administer entrectinib to the patient in the absence of adverse reaction or the absence of at least Grade 3 severity of adverse reaction in the patient or until cancer progression; or
if the patient experiences Grade 3 or Grade 4 severity of adverse reaction, withholding entrectinib and resuming administration of entrectinib at the initial dose or at a reduced dose upon patient recovery to Grade 1 or less severity of adverse reaction; or
permanently discontinuing administration of entrectinib if adverse reaction does not resolve within 4 weeks or Grade 4 severity adverse reactions recur.

Para. EB. A method of treating cancer in a patient in need thereof, the method comprising:
assessing left ventricular ejection fraction (LVEF) of the patient prior to administering entrectinib to the patient;
initiating administration of entrectinib to the patient with an initial dose;
monitoring the patient for clinical signs and symptoms of congestive heart failure (CHF), including shortness of breath and edema;
optionally obtaining a MRI or cardiac biopsy to make a diagnosis of congestive heart failure; and
continuing to administer entrectinib to the patient in the absence of adverse reaction or the absence of at least Grade 2 severity of congestive heart failure in the patient or until cancer progression; or
if the patient experiences Grade 2 or 3 severity of congestive heart failure, withholding administration of entrectinib, instituting appropriate medical management, reassessing LVEF and severity of congestive heart failure, and resuming administration of entrectinib at a reduced dose upon patient recovery to less than or equal to Grade 1 severity of congestive heart failure; or
permanently discontinuing administration of entrectinib if the patient experiences Grade 4 severity of congestive heart failure.

Para. EC. A method of treating cancer in a patient in need thereof, the method comprising:
initiating administration of entrectinib to the patient with an initial dose;
monitoring the patient for congestive heart failure; and
continuing to administer entrectinib to the patient in the absence of adverse reaction in the patient or until cancer progression; or
if the patient exhibits at least Grade 1 severity of congestive heart failure, withholding administration of entrectinib until patient recovery to less than or equal to Grade 1 severity of congestive heart failure; and resuming administration of entrectinib at a reduced dose; or permanently discontinuing administration of entrectinib to the patient.

Para. ED. A method of treating cancer in a patient in need thereof, the method comprising:
initiating administration of entrectinib to the patient with an initial dose;
monitoring the patient for congestive heart failure; and
continuing to administer entrectinib to the patient in the absence of adverse reaction or the absence of at least Grade 2 severity of congestive heart failure in the patient or until cancer progression; or
if the patient exhibits Grade 2 or Grade 3 severity of congestive heart failure, withholding administration of entrectinib until patient recovery to less than or equal to Grade 1 severity of congestive heart failure; and resuming administration of entrectinib at a reduced dose; or
if the patient exhibits Grade 4 severity of congestive heart failure, withholding administration of entrectinib until patient recovery to less than or equal to Grade 1 severity of congestive heart failure; and resuming administration of entrectinib at a reduced dose or permanently discontinuing administration of entrectinib to the patient.

Para. EE. A method of treating cancer in a patient in need thereof, the method comprising:
initiating administration of entrectinib to the patient with an initial dose;
monitoring the patient for cardiac disorders; and
continuing to administer entrectinib to the patient in the absence of adverse reaction or until cancer progression; or
if the patient exhibits at least Grade 1 severity of cardiac disorders (e.g., cardiac failure, ventricular extrasystoles, myocarditis, etc.), withholding administration of entrectinib until patient recovery to less than or equal to Grade 1 severity of cardiac disorders; and resuming administration of entrectinib at a reduced dose.

Para. EF. A method of treating cancer in a patient in need thereof, the method comprising:
prior to administration of entrectinib to the patient,
   advising the patient of a risk of one or more central nervous system (CNS) adverse reactions from the administration of entrectinib; and
   advising the patient not to drive or operate hazardous machinery if the patient experiences one or more central nervous system adverse reactions from the administration of entrectinib;
initiating administration of entrectinib to the patient with an initial dose;
monitoring the patient for one or more central nervous system adverse reactions; and
continuing to administer entrectinib to the patient in the absence of adverse reaction or the absence of at least Grade 2 severity of central nervous system adverse reaction in the patient or until cancer progression; or
if the patient experiences Grade 2 severity of central nervous system adverse reaction, withholding administration of entrectinib and resuming administration of entrectinib at the initial dose or at a reduced dose upon patient recovery to less than or equal to Grade 1 severity of central nervous system adverse reaction; or
if the patient experiences Grade 3 severity of central nervous system adverse reaction, withholding administration of entrectinib and resuming administration of entrectinib at the reduced dose upon patient recovery to less than or equal to Grade 1 severity of central nervous system adverse reaction; or
if the patient experiences Grade 4 severity of central nervous system adverse reaction, permanently discontinuing administration of entrectinib.

Para. EG. A method of treating cancer in a patient in need thereof, the method comprising:
initiating administration of entrectinib to the patient;
monitoring the patient for one or more signs or symptoms of skeletal fracture;
if the patient exhibits one or more signs or symptoms of skeletal fracture, promptly evaluating the patient and instituting appropriate medical management; and
continuing to administer entrectinib to the patient in the absence of additional adverse reaction in the patient or until cancer progression.

Para. EH. A method of treating cancer in a patient in need thereof, the method comprising:
initiating administration of entrectinib to the patient with an initial dose;
monitoring the patient with liver tests, including ALT and AST, every 2 weeks during the first month of administration, then monthly thereafter, and as clinically indicated; and
continuing to administer entrectinib to the patient in the absence of adverse reaction or the absence of at least Grade 3 severity of hepatotoxicity in the patient or until cancer progression; or
if the patient experiences Grade 3 severity of hepatotoxicity, withholding administration of entrectinib and resuming administration of entrectinib at the initial dose upon patient recovery within 4 weeks to less than or equal to Grade 1 severity of hepatotoxicity, or resuming administration of entrectinib at a reduced dose for recurrent Grade 3 severity of hepatotoxicity that resolves within 4 weeks; or permanently discontinuing administration of entrectinib if Grade 3 severity of hepatotoxicity does not resolve within 4 weeks; or
if the patient experiences Grade 4 severity of hepatotoxicity, withholding administration of entrectinib and resuming administration of entrectinib at the reduced dose upon patient recovery within 4 weeks to less than or equal to Grade 1 severity of hepatotoxicity, or permanently discontinuing administration of entrectinib if Grade 4 severity of hepatotoxicity does not resolve within 4 weeks or recurs; or
if the patient exhibits ALT or AST greater than 3 times the upper limit of normal (ULN) with concurrent total bilirubin greater than 1.5 times upper limit of normal in the absence of cholestasis or hemolysis, permanently discontinuing administration of entrectinib.

Para. EI. A method of treating cancer in a patient in need thereof, the method comprising:
initiating administration of entrectinib to the patient with an initial dose;
monitoring the patient for transaminase elevation; and
continuing to administer entrectinib to the patient in the absence of adverse reaction or the absence of at least Grade 3 severity of transaminase elevation in the patient or until cancer progression; or
if the patient exhibits Grade 3 severity of transaminase elevation, withholding administration of entrectinib and resuming administration of entrectinib to the patient without change in dose upon patient recovery within 4 weeks to less than or equal to Grade 1 severity of transaminase elevation, or resuming administration of entrectinib at a reduced dose for recurrent Grade 3 severity of transaminase elevation that resolve within 4 weeks, or permanently discontinuing administration of entrectinib to the patient if Grade 3 severity of transaminase elevation does not resolve within 4 weeks; or
if the patient exhibits Grade 4 severity of transaminase elevation, withholding administration of entrectinib and resuming administration of entrectinib to the patient without change in dose upon patient recovery within 4 weeks to less than or equal to Grade 1 severity of transaminase elevation, or permanently discontinuing administration of entrectinib to the patient if Grade 4 severity of transaminase elevation does not resolve within 4 weeks or recurs; or
if the patient exhibits ALT or AST elevation greater than 3 times the upper limit of normal with total bilirubin elevation being greater than 2 times upper limit of normal in the absence of cholestasis or hemolysis, permanently discontinuing administration of entrectinib to the patient.

Para. EJ. A method of treating cancer in a patient in need thereof, the method comprising:
initiating administration of entrectinib to the patient with an initial dose;
monitoring the patient for transaminase elevation; and
continuing to administer entrectinib to the patient in the absence of adverse reaction or the absence of at least Grade 3 severity of transaminase elevation in the patient or until cancer progression; or
if the patient exhibits Grade 3 severity of transaminase elevation, withholding administration of entrectinib and resuming administration of entrectinib to the patient without change in dose upon patient recovery within 4 weeks to less than or equal to Grade 1 severity of transaminase elevation, or resuming administration of entrectinib at a reduced dose for recurrent Grade 3 severity of transaminase elevation that resolves within 4 weeks, or permanently discontinuing administration of entrectinib to the patient if Grade 3 severity of transaminase elevation does not resolve within 4 weeks; or
if the patient exhibits Grade 4 severity of transaminase elevation, withholding administration of entrectinib and resuming administration of entrectinib to the patient at a reduced dose upon patient recovery within 4 weeks to less than or equal to Grade 1 severity of transaminase elevation, or permanently discontinuing administration of entrectinib to the patient if Grade 4 severity of transaminase elevation does not resolve within 4 weeks or recurs; or
if the patient exhibits ALT or AST elevation greater than 3 times the upper limit of normal with total bilirubin elevation being greater than 2 times upper limit of normal in the absence of cholestasis or hemolysis, permanently discontinuing administration of entrectinib to the patient.

Para. EK. A method of treating cancer in a patient in need thereof, the method comprising:
assessing serum uric acid levels in the patient prior to initiating administration of entrectinib;
initiating administration of entrectinib to the patient with an initial dose;
reassessing serum uric acid levels periodically during administration of entrectinib and monitoring the patient for signs and symptoms of hyperuricemia; and
continuing to administer entrectinib to the patient in the absence of adverse reaction in the patient or until cancer progression; or
if the patient is symptomatic of hyperuricemia or experiences Grade 4 hyperuricemia, initiating treatment with urate-lowering medications as clinically indicated, withholding entrectinib and resuming administration of entrectinib at the initial dose or at a reduced dose upon improvement of signs or symptoms of hyperuricemia.

Para. EL. A method of treating cancer in a patient in need thereof, the method comprising:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc greater than 500 ms, withholding entrectinib until QTc interval recovers to baseline and resuming administration of entrectinib at the initial dose or at a reduced dose; or
if the patient exhibits Torsade de pointes, polymorphic ventricular tachycardia, or signs or symptoms of serious arrhythmia, permanently discontinuing administration of entrectinib.

Para. EM. A method of treating cancer in a patient in need thereof, the method comprising:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QT interval; and
continuing to administer entrectinib to the patient in the absence of adverse reaction or the absence of at least Grade 2 severity of QT interval prolongation in the patient or until cancer progression; or
if the patient exhibits Grade 2 severity of QT interval prolongation, withholding entrectinib until patient recovery to baseline and resuming administration of entrectinib at the initial dose; or
if the patient exhibits Grade 3 or Grade 4 severity of QT interval prolongation, withholding entrectinib until patient recovery to baseline and resuming administration of entrectinib at a reduced dose; and permanently discontinuing administration of entrectinib if the patient exhibits signs and symptoms of serious arrhythmia.

Para. EN. A method of treating cancer in a patient in need thereof, the method comprising:
initiating administration of entrectinib to the patient with an initial dose;
continuing to administer entrectinib to the patient in the absence of adverse reaction or the absence of at least Grade 2 severity of QT interval prolongation in the patient or until cancer progression; or
if the patient exhibits Grade 2 severity of QT interval prolongation, withholding entrectinib until patient recovery to less than or equal to Grade 1 severity of QT interval prolongation and resuming administration of entrectinib at the initial dose; or
if the patient exhibits Grade 3 severity of QT interval prolongation, withholding entrectinib until patient recovery to less than or equal to Grade 1 severity of QT interval prolongation and resuming administration of entrectinib at a reduced dose; or withholding entrectinib until patient recovery within seven days to less than or equal to Grade 1 severity of QT interval prolongation and resuming administration of entrectinib at a reduced dose; or permanently discontinuing administration of entrectinib if there is no patient recovery within seven days to less than or equal to Grade 1 severity of QT interval prolongation; or
if the patient exhibits Grade 4 severity of QT interval prolongation, permanently discontinuing administration of entrectinib.

Para. EO. A method of treating cancer in a patient in need thereof, the method comprising:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or
if the patient exhibits Torsade de pointes, polymorphic ventricular tachycardia, or signs or symptoms of serious arrhythmia, permanently discontinuing administration of entrectinib.

Para. EP. A method of treating cancer in a patient in need thereof, the method comprising:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc of 481 to 500 ms, withholding entrectinib until QTc interval recovers to baseline and resuming administration of entrectinib at the initial dose; or
if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or
if the patient exhibits Torsade de pointes, polymorphic ventricular tachycardia, or signs or symptoms of serious arrhythmia, permanently discontinuing administration of entrectinib.

Para. EQ. A method of treating cancer in a patient in need thereof, the method comprising:
initiating administration of entrectinib to the patient with an initial dose;
monitoring the patient for a new vision disorder; and
continuing to administer entrectinib to the patient in the absence of adverse reaction or the absence of at least Grade 2 severity of vision disorder in the patient or until cancer progression; or
if the patient experiences Grade 2 or higher severity of vision disorder, withholding entrectinib until improvement or stabilization of the vision disorder, and resuming administration of entrectinib at the initial dose or at a reduced dose.

Para. ER. A method of treating cancer in a patient in need thereof, the method comprising:
initiating administration of entrectinib to the patient with an initial dose;
monitoring the patient for anemia or neutropenia; and
continuing to administer entrectinib to the patient in the absence of adverse reaction or the absence of at least Grade 3 severity of anemia or neutropenia in the patient or until cancer progression; or
if the patient experiences Grade 3 or Grade 4 severity of anemia or neutropenia, withholding entrectinib and resuming administration of entrectinib at the initial dose or at a reduced dose upon patient recovery to Grade 2 or less severity of anemia or neutropenia.

Para. ES. A method of treating cancer in a patient in need thereof, the method comprising:
initiating administration of entrectinib to the patient with an initial dose;
monitoring the patient for anemia or neutropenia; and
continuing to administer entrectinib to the patient in the absence of adverse reaction or the absence of at least Grade 3 severity of anemia or neutropenia in the patient or until cancer progression; or
if the patient experiences Grade 3 severity of anemia or neutropenia, withholding entrectinib and resuming administration of entrectinib at the initial dose or at a reduced dose upon patient recovery to Grade 2 or less severity of anemia or neutropenia; or
if the patient experiences Grade 4 severity of anemia or neutropenia, withholding entrectinib and resuming administration of entrectinib at a reduced dose upon patient recovery to Grade 2 or less severity of anemia or neutropenia.

Para. ET. A method of treating cancer in a patient in need thereof, the method comprising:
prior to the administration of entrectinib to the patient,
   advising the patient of a potential for cognitive change from the administration of entrectinib; and
   advising the patient not to drive or operate hazardous machinery if the patient experiences one or more symptoms of cognitive change from the administration of entrectinib;
initiating administration of entrectinib to the patient with an initial dose;
monitoring the patient for a cognitive disorder; and
continuing to administer entrectinib to the patient in the absence of adverse reaction or the absence of at least Grade 2 severity of cognitive disorder in the patient or until cancer progression; or
if the patient experiences an event of at least Grade 2 severity of cognitive disorder, withholding administration of entrectinib until patient recovery to less than or equal to Grade 1 severity of cognitive disorder and resuming administration of entrectinib at a reduced dose; or
if the patient experiences a recurrence of the event of at least Grade 2 severity of cognitive disorder, administering entrectinib at a further reduced dose; or
if the patient experiences prolonged, severe, or intolerable events of cognitive disorder, permanently discontinuing administration of entrectinib.

Para. EU. The method of Para. ET, wherein cognitive change or cognitive disorder comprises one or more of confusion, mental status changes, memory impairment, and hallucinations.

Para. EV. A method of treating cancer in a patient in need thereof, the method comprising:
initiating administration of entrectinib to the patient with an initial dose;
monitoring the patient for a cognitive disorder (e.g., confusion, mental status changes, memory impairment, hallucinations, dysarthria, etc.) or ataxia; and
continuing to administer entrectinib to the patient in the absence of adverse reaction or the absence of at least Grade 2 severity of cognitive disorder or ataxia in the patient or until cancer progression; or
if the patient experiences a first event of at least Grade 2 severity of cognitive disorder or ataxia, withholding administration of entrectinib until patient recovery to less than or equal to Grade 1 severity of cognitive disorder or ataxia and resuming administration of entrectinib at a reduced dose; or
if the patient experiences a recurrence of the event of at least Grade 2 severity of cognitive disorder or ataxia, administering entrectinib at a further reduced dose; or permanently discontinuing administration of entrectinib.

Para. EW. A method of treating cancer in a patient in need thereof, the method comprising:
prior to the administration of entrectinib to the patient,
   advising the patient of a potential for cognitive change from the administration of entrectinib; and
   advising the patient not to drive or operate hazardous machinery if the patient experiences one or more symptoms of cognitive change from the administration of entrectinib;
initiating administration of entrectinib to the patient with an initial dose;
monitoring the patient for a cognitive disorder; and
continuing to administer entrectinib to the patient in the absence of adverse reaction or the absence of at least Grade 2 severity of cognitive disorder in the patient or until cancer progression; or
if the patient experiences Grade 2 severity of cognitive disorder, withholding administration of entrectinib until patient recovery to less than or equal to Grade 1 severity of cognitive disorder and resuming administration of entrectinib at the initial dose or a reduced dose; or
if the patient experiences Grade 3 severity of cognitive disorder, withholding administration of entrectinib until patient recovery to less than or equal to Grade 1 severity of cognitive disorder and resuming administration of entrectinib at the reduced dose; or
if the patient experiences prolonged, severe, or intolerable events of cognitive disorder, permanently discontinuing administration of entrectinib.

Para. EX. A method of treating cancer in a patient in need thereof, the method comprising:
prior to the administration of entrectinib to the patient,
   advising the patient of a potential for embryo-fetal toxicity;
initiating administration of entrectinib to the patient; and
continuing to administer entrectinib to the patient in the absence of adverse reaction in the patient or until cancer progression; or
if the patient becomes pregnant, permanently discontinuing administration of entrectinib.

Para. EY. A method of treating cancer in a patient in need thereof, the method comprising:
initiating administration of entrectinib to the patient with an initial dose; and
monitoring the patient for adverse reaction; and
continuing to administer entrectinib to the patient in the absence of syncope in the patient or until cancer progression; or
if the patient experiences a first event of at least Grade 1 severity of syncope, withholding administration of entrectinib until patient recovery to baseline and resuming administration of entrectinib at a reduced dose; or
if the patient experiences a recurrence of at least Grade 1 severity of syncope, administering entrectinib at a further reduced dose; or permanently discontinuing administration of entrectinib.

Para. EZ. A method of treating cancer in a patient in need thereof, the method comprising:
initiating administration of entrectinib to the patient with an initial dose; and
monitoring the patient for interstitial lung disease; and
continuing to administer entrectinib to the patient in the absence of interstitial lung disease in the patient or until cancer progression; or
if the patient experiences a first event of Grade 1 or Grade 2 severity of interstitial lung disease, withholding administration of entrectinib until patient recovery to baseline and resuming administration of entrectinib at the initial dose; or
if the patient experiences a recurrence of Grade 1 or Grade 2 severity of interstitial lung disease or the patient experiences Grade 3 or Grade 4 severity of interstitial lung disease, permanently discontinuing administration of entrectinib.

Para. FA. A method of treating cancer or managing adverse reactions of administration of entrectinib in a patient in need thereof, the method comprising:
prior to administering entrectinib to the patient,
   (i) optionally advising the patient of a risk of one or more adverse reactions from the administration of entrectinib; and/or
   (ii) optionally assessing one or more parameters in the patient associated with an adverse reaction (e.g., LVEF for CHF, serum uric acid levels for hyperuricemia, QT interval and electrolytes for QT interval prolongation);
initiating administration of entrectinib to the patient with an initial dose (e.g., 600 mg orally per day);
monitoring the patient for adverse reaction; and
if the patient does not exhibit a severe adverse reaction, continuing to administer entrectinib until cancer progression; or
if the patient experiences a severe adverse reaction, withholding entrectinib and, upon patient recovery, resuming administration of entrectinib at the initial dose or at a reduced dose wherein patient recovery is either the absence of or a reduction of the severe adverse reaction or if the severe adverse reaction does not resolve within 4 weeks or if the severe adverse reaction recurs, permanently discontinuing administration of entrectinib.

Para. FB. The method of Para. FA, wherein the adverse reaction is clinical signs and symptoms of congestive heart failure, including shortness of breath and edema; and the severe adverse reaction is at least Grade 2 severity of congestive heart failure; wherein the one or more parameters comprise left ventricular ejection fraction (LVEF); wherein the method further comprises optionally obtaining a MRI or cardiac biopsy to make a diagnosis of congestive heart failure; and wherein if the patient experiences Grade 2 or 3 severity of congestive heart failure, the method comprises withholding administration of entrectinib, instituting appropriate medical management, reassessing LVEF and severity of congestive heart failure, and resuming administration of entrectinib at a reduced dose upon patient recovery or permanently discontinuing administration of entrectinib if the patient experiences Grade 4 severity of congestive heart failure.

Para. FC. The method of Para. FA, wherein the adverse reaction is congestive heart failure; and the severe adverse reaction is at least Grade 1 severity of congestive heart failure, wherein patient recovery is determined as less than or equal to a Grade 1 severity of congestive heart failure; and wherein if the patient exhibits at least Grade 1 severity of congestive heart failure, the method comprises withholding administration of entrectinib until patient recovery; and resuming administration of entrectinib at a reduced dose or permanently discontinuing administration of entrectinib to the patient.

Para. FD. The method of Para. FA, wherein the adverse reaction is congestive heart failure; and the severe adverse reaction is at least Grade 2 severity of congestive heart failure, and wherein patient recovery is determined as less than or equal to a Grade 1 severity of congestive heart failure; wherein if the patient exhibits at least Grade 2 severity of congestive heart failure, the method comprises withholding administration of entrectinib until patient recovery; and resuming administration of entrectinib at a reduced dose, or permanently discontinuing administration of entrectinib to the patient.

Para. FE. The method of Para. FA, wherein the adverse reaction is cardiac disorders (e.g., cardiac failure, ventricular extrasystoles, myocarditis, etc.); and the severe adverse reaction is at least Grade 1 severity of cardiac disorders, wherein patient recovery is determined as less than or equal to a Grade 1 severity of cardiac disorders; and wherein if the patient exhibits at least Grade 1 severity of cardiac disorders, the method comprises withholding administration of entrectinib until patient recovery; and resuming administration of entrectinib at a reduced dose or permanently discontinuing administration of entrectinib to the patient.

Para. FF. The method of Para. FA, wherein the adverse reactions is a central nervous system (CNS) adverse reaction; and the severe adverse reaction is at least Grade 2 severity of the central nervous system adverse reaction; and wherein patient recovery is determined as less than or equal to a Grade 1 severity of central nervous system adverse reaction; wherein prior to administration of entrectinib to the patient, the method further comprises advising the patient not to drive or operate hazardous machinery if the patient experiences the one or more central nervous system adverse reactions from the administration of entrectinib; and wherein if the patient experiences Grade 2 severity of central nervous system adverse reaction, the method comprises withholding administration of entrectinib and resuming administration of entrectinib at the initial dose or at a reduced dose upon patient recovery; or if the patient experiences Grade 3 severity of central nervous system adverse reaction, withholding administration of entrectinib and resuming administration of entrectinib at the reduced dose upon patient recovery; or if the patient experiences Grade 4 severity of central nervous system adverse reaction, permanently discontinuing administration of entrectinib.

Para. FG. The method of Para. FA, wherein the adverse reaction is skeletal fracture; and wherein if the patient exhibits one or more signs or symptoms of skeletal fracture, the method further comprises promptly evaluating the patient and instituting appropriate medical management.

Para. FH. The method of Para. FA, wherein the adverse reaction is hepatotoxicity; the monitoring comprises liver tests, including measurement of ALT and AST, every 2 weeks during the first month of administration, then monthly thereafter, and as clinically indicated; wherein the severe adverse reaction is at least a Grade 3 severity of hepatotoxicity; wherein patient recovery is determined as less than or equal to a Grade 1 severity of hepatotoxicity; and wherein if the patient experiences Grade 3 severity of hepatotoxicity, the method comprises withholding administration of entrectinib and resuming administration of entrectinib at the initial dose upon patient recovery within 4 weeks, or resuming administration of entrectinib at a reduced dose for recurrent Grade 3 severity of hepatotoxicity that resolve within 4 weeks; or permanently discontinuing administration of entrectinib if Grade 3 severity of hepatotoxicity does not resolve within 4 weeks; or if the patient experiences Grade 4 severity of hepatotoxicity, withholding administration of entrectinib and resuming administration of entrectinib at the reduced dose upon patient recovery within 4 weeks, or permanently discontinuing administration of entrectinib if Grade 4 severity of hepatotoxicity does not resolve within 4 weeks or recurs; or if the patient exhibits ALT or AST greater than 3 times the upper limit of normal (ULN) with concurrent total bilirubin greater than 1.5 times upper limit of normal in the absence of cholestasis or hemolysis, permanently discontinuing administration of entrectinib.

Para. FI. The method of Para. FA, wherein the adverse reaction is transaminase elevation; and the severe adverse reaction is at least a Grade 3 severity of transaminase elevation; wherein patient recovery is determined as less than or equal to a Grade 1 severity of transaminase elevation; and wherein if the patient exhibits Grade 3 severity of transaminase elevation, the method comprises withholding administration of entrectinib and resuming administration of entrectinib to the patient without change in dose upon patient recovery within 4 weeks, or resuming administration of entrectinib at a reduced dose for recurrent Grade 3 severity of transaminase elevation that resolve within 4 weeks, or permanently discontinuing administration of entrectinib to the patient if Grade 3 severity of transaminase elevation does not resolve within 4 weeks; or if the patient exhibits Grade 4 severity of transaminase elevation, withholding administration of entrectinib and resuming administration of entrectinib to the patient without change in dose upon patient recovery within 4 weeks, or permanently discontinuing administration of entrectinib to the patient if Grade 4 severity of transaminase elevation does not resolve within 4 weeks or recurs; or if the patient exhibits ALT or AST elevation greater than 3 times the upper limit of normal with total bilirubin elevation being greater than 2 times upper limit of normal in the absence of cholestasis or hemolysis, permanently discontinuing administration of entrectinib to the patient.

Para. FJ. The method of Para. FA, wherein the adverse reaction is transaminase elevation; and the severe adverse reaction is at least a Grade 3 severity of transaminase elevation; wherein patient recovery is determined as less than or equal to a Grade 1 severity of transaminase elevation; and wherein if the patient exhibits Grade 3 severity of transaminase elevation, the method comprises withholding administration of entrectinib until patient recovery; and resuming administration of entrectinib to the patient without change in dose upon patient recovery within 4 weeks, or resuming administration of entrectinib at a reduced dose for recurrent Grade 3 severity of transaminase elevation that resolve within 4 weeks, or permanently discontinuing administration of entrectinib to the patient if Grade 3 severity of transaminase elevation does not resolve within 4 weeks; or if the patient exhibits Grade 4 severity of transaminase elevation, withholding administration of entrectinib until patient recovery; and resuming administration of entrectinib to the patient at a reduced dose upon patient recovery within 4 weeks, or permanently discontinuing administration of entrectinib to the patient if Grade 4 severity of transaminase elevation does not resolve within 4 weeks or recurs; or if the patient exhibits ALT or AST elevation greater than 3 times the upper limit of normal with total bilirubin elevation being greater than 2 times upper limit of normal in the absence of cholestasis or hemolysis, permanently discontinuing administration of entrectinib to the patient.

Para. FK. The method of Para. FA, wherein the adverse reaction is hyperuricemia; wherein the one or more parameters comprise serum uric acid levels in the patient; wherein the method further comprises reassessing serum uric acid levels periodically during administration of entrectinib; wherein the severe adverse reaction is symptoms of hyperuricemia or Grade 4 hyperuricemia; and wherein if the patient is symptomatic of hyperuricemia or experiences Grade 4 hyperuricemia, the method comprises initiating treatment with urate-lowering medications as clinically indicated, withholding entrectinib and resuming administration of entrectinib at the initial dose or at a reduced dose upon improvement of signs or symptoms of hyperuricemia.

Para. FL. The method of Para. FA, wherein the adverse reaction is prolongation of the QTc interval; wherein the one or more parameters comprise QT interval and electrolytes in the patient; wherein the method further comprises reassessing QT interval and electrolytes periodically during administration of entrectinib, adjusting frequency based upon patient's risk factors such as congestive heart failure, electrolyte abnormalities, or concomitant medications known to prolong the QTc interval; and wherein if the patient exhibits QTc greater than 500 ms, the method further comprises withholding entrectinib until QTc interval recovers to baseline and resuming administration of entrectinib at the initial dose or at a reduced dose; or if the patient exhibits Torsade de pointes, polymorphic ventricular tachycardia, or signs or symptoms of serious arrhythmia, permanently discontinuing administration of entrectinib.

Para. FM. The method of Para. FA, wherein the adverse reaction is QT interval prolongation; and the severe adverse reaction is at least a Grade 2 severity of QT interval prolongation; wherein the one or more parameters comprise QT interval and electrolytes in the patient; wherein the method further comprises reassessing QT interval and electrolytes periodically during administration of entrectinib, adjusting frequency based upon patient's risk factors such as congestive heart failure, electrolyte abnormalities, or concomitant medications known to prolong the QT interval; and wherein if the patient exhibits Grade 2 severity of QT interval prolongation, the method comprises withholding entrectinib until patient recovery to baseline and resuming administration of entrectinib at the initial dose; or if the patient exhibits Grade 3 or Grade 4 severity of QT interval prolongation, withholding entrectinib until patient recovery to baseline and resuming administration of entrectinib at a reduced dose; and permanently discontinuing administration of entrectinib if the patient exhibits signs and symptoms of serious arrhythmia.

Para. FN. The method of Para. FA, wherein the adverse reaction is QT interval prolongation; and the severe adverse reaction is at least a Grade 2 severity of QT interval prolongation; wherein patient recovery is determined as less than or equal to a Grade 1 severity of QT interval prolongation; and wherein if the patient exhibits Grade 2 severity of QT interval prolongation, the method comprises withholding entrectinib until patient recovery and resuming administration of entrectinib at the initial dose; or if the patient exhibits Grade 3 severity of QT interval prolongation, withholding entrectinib until patient recovery and resuming administration of entrectinib at a reduced dose; or withholding entrectinib until patient recovery within seven days and resuming administration of entrectinib at a reduced dose; or permanently discontinuing administration of entrectinib if there is no patient recovery within seven days; or if the patient exhibits Grade 4 severity of QT interval prolongation, permanently discontinuing administration of entrectinib.

Para. FO. The method of Para. FA, wherein the adverse reaction is prolongation of the QTc interval; wherein the one or more parameters comprise QT interval and electrolytes in the patient; wherein the method further comprises reassessing QT interval and electrolytes periodically during administration of entrectinib, adjusting frequency based upon patient's risk factors such as congestive heart failure, electrolyte abnormalities, or concomitant medications known to prolong the QTc interval; and wherein if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or if the patient exhibits Torsade de pointes, polymorphic ventricular tachycardia, or signs or symptoms of serious arrhythmia, permanently discontinuing administration of entrectinib.

Para. FP. The method of Para. FA, wherein the adverse reaction is prolongation of the QTc interval; wherein the one or more parameters comprise QT interval and electrolytes in the patient; wherein the method further comprises reassessing QT interval and electrolytes periodically during administration of entrectinib, adjusting frequency based upon patient's risk factors such as congestive heart failure, electrolyte abnormalities, or concomitant medications known to prolong the QTc interval; and wherein if the patient exhibits QTc of 481 to 500 ms, the method further comprises withholding entrectinib until QTc interval recovers to baseline and resuming administration of entrectinib at the initial dose; or if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or if the patient exhibits Torsade de pointes, polymorphic ventricular tachycardia, or signs or symptoms of serious arrhythmia, permanently discontinuing administration of entrectinib.

Para. FQ. The method of Para. FA, wherein the adverse reaction is a vision disorder; and the severe adverse reaction is at least a Grade 2 severity of vision disorder; and wherein if the patient experiences Grade 2 or higher severity of vision disorder, the method comprises withholding entrectinib until improvement or stabilization of the vision disorder, and resuming administration of entrectinib at the initial dose or at a reduced dose.

Para. FR. The method of Para. FA, wherein the adverse reaction is anemia or neutropenia; and the severe adverse reaction is at least a Grade 3 severity of anemia or neutropenia; and wherein if the patient experiences Grade 3 or Grade 4 severity of anemia or neutropenia, the method comprises withholding entrectinib and resuming administration of entrectinib at the initial dose or at a reduced dose upon patient recovery to Grade 2 or less severity of anemia or neutropenia.

Para. FS. The method of Para. FA, wherein the adverse reaction is anemia or neutropenia; and the severe adverse reaction is at least a Grade 3 severity of anemia or neutropenia; wherein patient recovery is determined as less than or equal to a Grade 2 severity of anemia or neutropenia; and wherein if the patient experiences Grade 3 severity of anemia or neutropenia, the method comprises withholding entrectinib and resuming administration of entrectinib at the initial dose or at a reduced dose upon patient recovery; or if the patient experiences Grade 4 severity of anemia or neutropenia, withholding entrectinib and resuming administration of entrectinib at a reduced dose upon patient recovery.

Para. FT. The method of Para. FA, wherein the adverse reaction is a cognitive disorder; and the severe adverse reaction is at least a Grade 2 severity of cognitive disorder; wherein patient recovery is determined as less than or equal to a Grade 1 severity of cognitive disorder; wherein prior to administration of entrectinib to the patient, the method further comprises advising the patient of a potential for cognitive change from the administration of entrectinib and advising the patient not to drive or operate hazardous machinery if the patient experiences one or more symptoms of cognitive change from the administration of entrectinib; and wherein if the patient experiences an event of at least Grade 2 severity of cognitive disorder, the method comprises withholding administration of entrectinib until patient recovery and resuming administration of entrectinib at a reduced dose; or if the patient experiences a recurrence of the event of at least Grade 2 severity of cognitive disorder, administering entrectinib at a further reduced dose; or if the patient experiences prolonged, severe, or intolerable events of cognitive disorder, permanently discontinuing administration of entrectinib.

Para. FU. The method of Para. FT, wherein cognitive change or cognitive disorder comprises one or more of confusion, mental status changes, memory impairment, and hallucinations.

Para. FV. The method of Para. FA, wherein the adverse reaction is a cognitive disorder or ataxia; and the severe adverse reaction is at least a Grade 2 severity of cognitive disorder or ataxia; wherein patient recovery is determined as less than or equal to a Grade 1 severity of cognitive disorder or ataxia; and wherein if the patient experiences a first event of at least Grade 2 severity of cognitive disorder or ataxia, the method comprises withholding administration of entrectinib until patient recovery and resuming administration of entrectinib at a reduced dose; or if the patient experiences a recurrence of the event of at least Grade 2 severity of cognitive disorder or ataxia, administering entrectinib at a further reduced dose; or permanently discontinuing administration of entrectinib.

Para. FW. The method of Para. FA, wherein the adverse reaction is a cognitive disorder; and the severe adverse reaction is at least a Grade 2 severity of cognitive disorder; wherein patient recovery is determined as less than or equal to a Grade 1 severity of cognitive disorder; wherein prior to administration of entrectinib to the patient, the method further comprises advising the patient of a potential for cognitive change from the administration of entrectinib and advising the patient not to drive or operate hazardous machinery if the patient experiences one or more symptoms of cognitive change from the administration of entrectinib; and wherein if the patient experiences an event of at least Grade 2 severity of cognitive disorder, the method comprises withholding administration of entrectinib until patient recovery and resuming administration of entrectinib at the initial dose or a reduced dose; or if the patient experiences Grade 3 severity of cognitive disorder, withholding administration of entrectinib until patient recovery and resuming administration of entrectinib at a reduced dose; or if the patient experiences prolonged, severe, or intolerable events of cognitive disorder, permanently discontinuing administration of entrectinib.

Para. FX. The method of Para. FA, wherein the adverse reaction is embryo-fetal toxicity; and wherein if the patient becomes pregnant, the method further comprises permanently discontinuing administration of entrectinib.

Para. FY. The method of Para. FA, wherein the adverse reaction is syncope; the severe adverse reaction is at least a Grade 1 severity of syncope; wherein patient recovery is determined as recovery to baseline; and wherein if the patient experiences a first event of at least Grade 1 severity of syncope, the method comprises withholding administration of entrectinib until patient recovery and resuming administration of entrectinib at a reduced dose; or if the patient experiences a recurrence of at least Grade 1 severity of syncope, administering entrectinib at a further reduced dose; or permanently discontinuing administration of entrectinib.

Para. FZ. The method of Para. FA, wherein the adverse reaction is interstitial lung disease; the severe adverse reaction is at least a Grade 1 severity of interstitial lung disease; wherein patient recovery is determined as recovery to baseline; and wherein if the patient experiences a first event of Grade 1 or Grade 2 severity of syncope, the method comprises withholding administration of entrectinib until patient recovery and resuming administration of entrectinib at the initial dose; or if the patient experiences a recurrence of Grade 1 or Grade 2 severity of interstitial lung disease or the patient experiences Grade 3 or Grade 4 severity of interstitial lung disease, permanently discontinuing administration of entrectinib.

Para. GA. The method of Para. FA, wherein the adverse reaction is congestive heart failure, a central nervous system (CNS) adverse reaction, fracture (e.g., skeletal fracture), hepatotoxicity, hyperuricemia, QT interval prolongation, a vision disorder, anemia/neutropenia, a cognitive disorder, transaminase elevation, or an embryo-fetal toxicity.

Para. HA. A method of managing adverse reactions of administration of entrectinib in a patient, the method comprising:
initiating administration of entrectinib to the patient with an initial dose;
monitoring the patient for adverse reaction; and
continuing to administer entrectinib to the patient in the absence of adverse reaction or the absence of at least Grade 3 severity of adverse reaction in the patient or until cancer progression; or
if the patient experiences Grade 3 or Grade 4 severity of adverse reaction, withholding entrectinib and resuming administration of entrectinib at the initial dose or at a reduced dose upon patient recovery to Grade 1 or less severity of adverse reaction or permanently discontinuing administration of entrectinib if adverse reaction does not resolve within 4 weeks or Grade 4 severity adverse reactions recur.

Para. HB. A method of managing adverse reactions of administration of entrectinib in a patient, the method comprising:
assessing left ventricular ejection fraction (LVEF) of the patient prior to administering entrectinib to the patient;
initiating administration of entrectinib to the patient with an initial dose;
monitoring the patient for clinical signs and symptoms of congestive heart failure (CHF), including shortness of breath and edema;
optionally obtaining a MRI or cardiac biopsy to make a diagnosis of congestive heart failure; and
continuing to administer entrectinib to the patient in the absence of adverse reaction or the absence of at least Grade 2 severity of congestive heart failure in the patient or until cancer progression; or
if the patient experiences Grade 2 or 3 severity of congestive heart failure, withholding administration of entrectinib, instituting appropriate medical management, reassessing LVEF and severity of congestive heart failure, and resuming administration of entrectinib at a reduced dose upon patient recovery to less than or equal to Grade 1 severity of congestive heart failure or permanently discontinuing administration of entrectinib if the patient experiences Grade 4 severity of congestive heart failure.

Para. HC. A method of managing adverse reactions of administration of entrectinib in a patient, the method comprising:
initiating administration of entrectinib to the patient with an initial dose;
monitoring the patient for congestive heart failure; and
continuing to administer entrectinib to the patient in the absence of adverse reaction in the patient or until cancer progression; or
if the patient exhibits at least Grade 1 severity of congestive heart failure, withholding administration of entrectinib until patient recovery to less than or equal to Grade 1 severity of congestive heart failure; and resuming administration of entrectinib at a reduced dose; or permanently discontinuing administration of entrectinib to the patient

Para. HD. A method of managing adverse reactions of administration of entrectinib in a patient, the method comprising:
initiating administration of entrectinib to the patient with an initial dose;
monitoring the patient for congestive heart failure; and
continuing to administer entrectinib to the patient in the absence of adverse reaction or the absence of at least Grade 2 severity of congestive heart failure in the patient or until cancer progression; or
if the patient exhibits Grade 2 or Grade 3 severity of congestive heart failure, withholding administration of entrectinib until patient recovery to less than or equal to Grade 1 severity of congestive heart failure; and resuming administration of entrectinib at a reduced dose; or
if the patient exhibits Grade 4 severity of congestive heart failure, withholding administration of entrectinib until patient recovery to less than or equal to Grade 1 severity of congestive heart failure; and resuming administration of entrectinib at a reduced dose or permanently discontinuing administration of entrectinib to the patient.

Para. HE. A method of managing adverse reactions of administration of entrectinib in a patient, the method comprising:
initiating administration of entrectinib to the patient with an initial dose;
monitoring the patient for cardiac disorders; and
continuing to administer entrectinib to the patient in the absence of adverse reaction or until cancer progression; or
if the patient exhibits at least Grade 1 severity of cardiac disorders (e.g., cardiac failure, ventricular extrasystoles, myocarditis, etc.), withholding administration of entrectinib until patient recovery to less than or equal to Grade 1 severity of cardiac disorders; and resuming administration of entrectinib at a reduced dose.

Para. HF. A method of managing adverse reactions of administration of entrectinib in a patient, the method comprising:
prior to administration of entrectinib to the patient,
   advising the patient of a risk of one or more central nervous system (CNS) adverse reactions from the administration of entrectinib; and
   advising the patient not to drive or operate hazardous machinery if the patient experiences one or more central nervous system adverse reactions from the administration of entrectinib;
initiating administration of entrectinib to the patient with an initial dose;
monitoring the patient for one or more central nervous system adverse reactions; and
continuing to administer entrectinib to the patient in the absence of adverse reaction or the absence of at least Grade 2 severity of central nervous system adverse reaction in the patient or until cancer progression; or
if the patient experiences Grade 2 severity of central nervous system adverse reaction, withholding administration of entrectinib and resuming administration of entrectinib at the initial dose or at a reduced dose upon patient recovery to less than or equal to Grade 1 severity of central nervous system adverse reaction; or
if the patient experiences Grade 3 severity of central nervous system adverse reaction, withholding administration of entrectinib and resuming administration of entrectinib at the reduced dose upon patient recovery to less than or equal to Grade 1 severity of central nervous system adverse reaction; or
if the patient experiences Grade 4 severity of central nervous system adverse reaction, permanently discontinuing administration of entrectinib.

Para. HG. A method of managing adverse reactions of administration of entrectinib in a patient, the method comprising:
initiating administration of entrectinib to the patient;
monitoring the patient for one or more signs or symptoms of skeletal fracture;
if the patient exhibits one or more signs or symptoms of skeletal fracture, promptly evaluating the patient and instituting appropriate medical management; and
continuing to administer entrectinib to the patient in the absence of additional adverse reaction in the patient or until cancer progression.

Para. HH. A method of managing adverse reactions of administration of entrectinib in a patient, the method comprising:
initiating administration of entrectinib to the patient with an initial dose;
monitoring the patient with liver tests, including ALT and AST, every 2 weeks during the first month of administration, then monthly thereafter, and as clinically indicated; and
continuing to administer entrectinib to the patient in the absence of adverse reaction or the absence of at least Grade 3 severity of hepatotoxicity in the patient or until cancer progression; or
if the patient experiences Grade 3 severity of hepatotoxicity, withholding administration of entrectinib and resuming administration of entrectinib at the initial dose upon patient recovery within 4 weeks to less than or equal to Grade 1 severity of hepatotoxicity, or resuming administration of entrectinib at a reduced dose for recurrent Grade 3 severity of hepatotoxicity that resolve within 4 weeks; or permanently discontinuing administration of entrectinib if Grade 3 severity of hepatotoxicity does not resolve within 4 weeks; or
if the patient experiences Grade 4 severity of hepatotoxicity, withholding administration of entrectinib and resuming administration of entrectinib at the reduced dose upon patient recovery within 4 weeks to less than or equal to Grade 1 severity of hepatotoxicity, or permanently discontinuing administration of entrectinib if Grade 4 severity of hepatotoxicity does not resolve within 4 weeks or recurs; or
if the patient exhibits ALT or AST greater than 3 times the upper limit of normal (ULN) with concurrent total bilirubin greater than 1.5 times upper limit of normal in the absence of cholestasis or hemolysis, permanently discontinuing administration of entrectinib.

Para. HI. A method of managing adverse reactions of administration of entrectinib in a patient, the method comprising:
initiating administration of entrectinib to the patient with an initial dose;
monitoring the patient for transaminase elevation; and
continuing to administer entrectinib to the patient in the absence of adverse reaction or the absence of at least Grade 3 severity of transaminase elevation in the patient or until cancer progression; or
if the patient exhibits Grade 3 severity of transaminase elevation, withholding administration of entrectinib and resuming administration of entrectinib to the patient without change in dose upon patient recovery within 4 weeks to less than or equal to Grade 1 severity of transaminase elevation, or resuming administration of entrectinib at a reduced dose for recurrent Grade 3 severity of transaminase elevation that resolve within 4 weeks, or permanently discontinuing administration of entrectinib to the patient if Grade 3 severity of transaminase elevation does not resolve within 4 weeks; or
if the patient exhibits Grade 4 severity of transaminase elevation, withholding administration of entrectinib and resuming administration of entrectinib to the patient without change in dose upon patient recovery within 4 weeks to less than or equal to Grade 1 severity of transaminase elevation, or permanently discontinuing administration of entrectinib to the patient if Grade 4 severity of transaminase elevation does not resolve within 4 weeks or recurs; or
if the patient exhibits ALT or AST elevation greater than 3 times the upper limit of normal with total bilirubin elevation being greater than 2 times upper limit of normal in the absence of cholestasis or hemolysis, permanently discontinuing administration of entrectinib to the patient.

Para. HJ. A method of managing adverse reactions of administration of entrectinib in a patient, the method comprising:
initiating administration of entrectinib to the patient with an initial dose;
monitoring the patient for transaminase elevation; and
continuing to administer entrectinib to the patient in the absence of adverse reaction or the absence of at least Grade 3 severity of transaminase elevation in the patient or until cancer progression; or
if the patient exhibits Grade 3 severity of transaminase elevation, withholding administration of entrectinib and resuming administration of entrectinib to the patient without change in dose upon patient recovery within 4 weeks to less than or equal to Grade 1 severity of transaminase elevation, or resuming administration of entrectinib at a reduced dose for recurrent Grade 3 severity of transaminase elevation that resolve within 4 weeks, or permanently discontinuing administration of entrectinib to the patient if Grade 3 severity of transaminase elevation does not resolve within 4 weeks; or
if the patient exhibits Grade 4 severity of transaminase elevation, withholding administration of entrectinib and resuming administration of entrectinib to the patient at a reduced dose upon patient recovery within 4 weeks to less than or equal to Grade 1 severity of transaminase elevation, or permanently discontinuing administration of entrectinib to the patient if Grade 4 severity of transaminase elevation does not resolve within 4 weeks or recurs; or
if the patient exhibits ALT or AST elevation greater than 3 times the upper limit of normal with total bilirubin elevation being greater than 2 times upper limit of normal in the absence of cholestasis or hemolysis, permanently discontinuing administration of entrectinib to the patient.

Para. HK. A method of managing adverse reactions of administration of entrectinib in a patient, the method comprising:
assessing serum uric acid levels in the patient prior to initiating administration of entrectinib;
initiating administration of entrectinib to the patient with an initial dose;
reassessing serum uric acid levels periodically during administration of entrectinib and monitoring the patient for signs and symptoms of hyperuricemia; and
continuing to administer entrectinib to the patient in the absence of adverse reaction in the patient or until cancer progression; or
if the patient is symptomatic of hyperuricemia or experiences Grade 4 hyperuricemia, initiating treatment with urate-lowering medications as clinically indicated, withholding entrectinib and resuming administration of entrectinib at the initial dose or at a reduced dose upon improvement of signs or symptoms of hyperuricemia.

Para. HL. A method of managing adverse reactions of administration of entrectinib in a patient, the method comprising:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc greater than 500 ms, withholding entrectinib until QTc interval recovers to baseline and resuming administration of entrectinib at the initial dose or at a reduced dose; or
if the patient exhibits Torsade de pointes, polymorphic ventricular tachycardia, or signs or symptoms of serious arrhythmia, permanently discontinuing administration of entrectinib.

Para. HM. A method of managing adverse reactions of administration of entrectinib in a patient, the method comprising
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QT interval; and
continuing to administer entrectinib to the patient in the absence of adverse reaction or the absence of at least Grade 2 severity of QT interval prolongation in the patient or until cancer progression; or
if the patient exhibits Grade 2 severity of QT interval prolongation, withholding entrectinib until patient recovery to baseline and resuming administration of entrectinib at the initial dose; or
if the patient exhibits Grade 3 or Grade 4 severity of QT interval prolongation, withholding entrectinib until patient recovery to baseline and resuming administration of entrectinib at a reduced dose; and permanently discontinuing administration of entrectinib if the patient exhibits signs and symptoms of serious arrhythmia.

Para. HN. A method of managing adverse reactions of administration of entrectinib in a patient, the method comprising:
initiating administration of entrectinib to the patient with an initial dose;
continuing to administer entrectinib to the patient in the absence of adverse reaction or the absence of at least Grade 2 severity of QT interval prolongation in the patient or until cancer progression; or
if the patient exhibits Grade 2 severity of QT interval prolongation, withholding entrectinib until patient recovery to less than or equal to Grade 1 severity of QT interval prolongation and resuming administration of entrectinib at the initial dose; or
if the patient exhibits Grade 3 severity of QT interval prolongation, withholding entrectinib until patient recovery to less than or equal to Grade 1 severity of QT interval prolongation and resuming administration of entrectinib at a reduced dose; or withholding entrectinib until patient recovery within seven days to less than or equal to Grade 1 severity of QT interval prolongation and resuming administration of entrectinib at a reduced dose; or permanently discontinuing administration of entrectinib if there is no patient recovery within seven days to less than or equal to Grade 1 severity of QT interval prolongation; or
if the patient exhibits Grade 4 severity of QT interval prolongation, permanently discontinuing administration of entrectinib.

Para. HO. A method of managing adverse reactions of administration of entrectinib in a patient, the method comprising:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or
if the patient exhibits Torsade de pointes, polymorphic ventricular tachycardia, or signs or symptoms of serious arrhythmia, permanently discontinuing administration of entrectinib.

Para. HP. A method of managing adverse reactions of administration of entrectinib in a patient, the method comprising:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc of 481 to 500 ms, withholding entrectinib until QTc interval recovers to baseline and resuming administration of entrectinib at the initial dose; or
if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or
if the patient exhibits Torsade de pointes, polymorphic ventricular tachycardia, or signs or symptoms of serious arrhythmia, permanently discontinuing administration of entrectinib.

Para. HQ. A method of managing adverse reactions of administration of entrectinib in a patient, the method comprising:
initiating administration of entrectinib to the patient with an initial dose;
monitoring the patient for a new vision disorder; and
continuing to administer entrectinib to the patient in the absence of adverse reaction or the absence of at least Grade 2 severity of vision disorder in the patient or until cancer progression; or
if the patient experiences Grade 2 or higher severity of vision disorder, withholding entrectinib until improvement or stabilization of the vision disorder, and resuming administration of entrectinib at the initial dose or at a reduced dose.

Para. HR. A method of managing adverse reactions of administration of entrectinib in a patient, the method comprising:
initiating administration of entrectinib to the patient with an initial dose;
monitoring the patient for anemia or neutropenia; and
continuing to administer entrectinib to the patient in the absence of adverse reaction or the absence of at least Grade 3 severity of adverse reaction in the patient or until cancer progression; or
if the patient experiences Grade 3 or Grade 4 severity of anemia or neutropenia, withholding entrectinib and resuming administration of entrectinib at the initial dose or at a reduced dose upon patient recovery to Grade 2 or less severity of anemia or neutropenia.

Para. HS. A method of managing adverse reactions of administration of entrectinib in a patient, the method comprising:
initiating administration of entrectinib to the patient with an initial dose;
monitoring the patient for anemia or neutropenia; and
continuing to administer entrectinib to the patient in the absence of adverse reaction or the absence of at least Grade 3 severity of anemia or neutropenia in the patient or until cancer progression; or
if the patient experiences Grade 3 severity of anemia or neutropenia, withholding entrectinib and resuming administration of entrectinib at the initial dose or at a reduced dose upon patient recovery to Grade 2 or less severity of anemia or neutropenia; or
if the patient experiences Grade 4 severity of anemia or neutropenia, withholding entrectinib and resuming administration of entrectinib at a reduced dose upon patient recovery to Grade 2 or less severity of anemia or neutropenia.

Para. HT. A method of managing adverse reactions of administration of entrectinib in a patient, the method comprising:
prior to the administration of entrectinib to the patient,
   advising the patient of a potential for cognitive change from the administration of entrectinib; and
   advising the patient not to drive or operate hazardous machinery if the patient experiences one or more symptoms of cognitive change from the administration of entrectinib;
initiating administration of entrectinib to the patient with an initial dose;
monitoring the patient for a cognitive disorder; and
continuing to administer entrectinib to the patient in the absence of adverse reaction or the absence of at least Grade 2 severity of cognitive disorder in the patient or until cancer progression; or
if the patient experiences an event of at least Grade 2 severity of cognitive disorder, withholding administration of entrectinib until patient recovery to less than or equal to Grade 1 severity of cognitive disorder and resuming administration of entrectinib at a reduced dose; or
if the patient experiences a recurrence of the event of at least Grade 2 severity of cognitive disorder, administering entrectinib at a further reduced dose; or
if the patient experiences prolonged, severe, or intolerable events of cognitive disorder, permanently discontinuing administration of entrectinib.

Para. HU. The method of Para. HT, wherein cognitive change or cognitive disorder comprises one or more of confusion, mental status changes, memory impairment, and hallucinations.

Para. HV. A method of managing adverse reactions of administration of entrectinib in a patient, the method comprising:
initiating administration of entrectinib to the patient with an initial dose;
monitoring the patient for a cognitive disorder (e.g., confusion, mental status changes, memory impairment, hallucinations, dysarthria, etc.) or ataxia; and
continuing to administer entrectinib to the patient in the absence of adverse reaction or the absence of at least Grade 2 severity of cognitive disorder or ataxia in the patient or until cancer progression; or
if the patient experiences a first event of at least Grade 2 severity of cognitive disorder or ataxia, withholding administration of entrectinib until patient recovery to less than or equal to Grade 1 severity of cognitive disorder or ataxia and resuming administration of entrectinib at a reduced dose; or
if the patient experiences a recurrence of the event of at least Grade 2 severity of cognitive disorder or ataxia, administering entrectinib at a further reduced dose; or permanently discontinuing administration of entrectinib.

Para. HW. A method of managing adverse reactions of administration of entrectinib in a patient, the method comprising:
prior to the administration of entrectinib to the patient,
   advising the patient of a potential for cognitive change from the administration of entrectinib; and
   advising the patient not to drive or operate hazardous machinery if the patient experiences one or more symptoms of cognitive change from the administration of entrectinib;
initiating administration of entrectinib to the patient with an initial dose;
monitoring the patient for a cognitive disorder; and
continuing to administer entrectinib to the patient in the absence of adverse reaction or the absence of at least Grade 2 severity of cognitive disorder in the patient or until cancer progression; or
if the patient experiences Grade 2 severity of cognitive disorder, withholding administration of entrectinib until patient recovery to less than or equal to Grade 1 severity of cognitive disorder and resuming administration of entrectinib at the initial dose or a reduced dose; or
if the patient experiences Grade 3 severity of cognitive disorder, withholding administration of entrectinib until patient recovery to less than or equal to Grade 1 severity of cognitive disorder and resuming administration of entrectinib at a reduced dose; or
if the patient experiences prolonged, severe, or intolerable events of cognitive disorder, permanently discontinuing administration of entrectinib.

Para. HX. A method of managing adverse reactions of administration of entrectinib in a patient, the method comprising:
prior to the administration of entrectinib to the patient,
   advising the patient of a potential for embryo-fetal toxicity;
initiating administration of entrectinib to the patient; and
continuing to administer entrectinib to the patient in the absence of adverse reaction in the patient or until cancer progression; or
if the patient becomes pregnant, permanently discontinuing administration of entrectinib.

Para. HY. A method of managing adverse reactions of administration of entrectinib in a patient, the method comprising:
initiating administration of entrectinib to the patient with an initial dose; and
monitoring the patient for adverse reaction; and
continuing to administer entrectinib to the patient in the absence of syncope in the patient or until cancer progression; or
if the patient experiences a first event of at least Grade 1 severity of syncope, withholding administration of entrectinib until patient recovery to baseline and resuming administration of entrectinib at a reduced dose; or
if the patient experiences a recurrence of at least Grade 1 severity of syncope, administering entrectinib at a further reduced dose; or permanently discontinuing administration of entrectinib.

Para. HZ. A method of managing adverse reactions of administration of entrectinib in a patient, the method comprising:
initiating administration of entrectinib to the patient with an initial dose; and
monitoring the patient for interstitial lung disease; and
continuing to administer entrectinib to the patient in the absence of interstitial lung disease in the patient or until cancer progression; or
if the patient experiences a first event of Grade 1 or Grade 2 severity of interstitial lung disease, withholding administration of entrectinib until patient recovery to baseline and resuming administration of entrectinib at the initial dose; or
if the patient experiences a recurrence of Grade 1 or Grade 2 severity of interstitial lung disease or the patient experiences Grade 3 or Grade 4 severity of interstitial lung disease, permanently discontinuing administration of entrectinib.

Para. IA. The method of any one of Paras. EA-EF, EH-EW, EY, EZ, FA-GA, HA-HF, HH-HW, HY, or HZ, wherein the initial dose is 600 mg, orally administered once daily, and the reduced dose is 400 mg, orally administered once daily.

Para. IB. The method of any one of Paras. EA-EF, EH-EW, EY, EZ, FA-GA, HA-HF, HH-HW, HY, or HZ, wherein the initial dose is 100 mg, orally administered once daily, and the reduced dose is 100 mg, orally administered once per day for 5 days each week.

Para. IC. The method of any one of Paras. EA-EF, EH-EW, EY, EZ, FA-GA, HA-HF, HH-HW, HY, or HZ, wherein the initial dose is 200 mg, orally administered once daily, and the reduced dose is 200 mg, orally administered once per day for 5 days each week.

Para. ID. The method of any one of Paras. EA-EF, EH-EW, EY, EZ, FA-GA, HA-HF, HH-HW, HY, or HZ, wherein the initial dose is 300 mg, orally administered once daily, and the reduced dose is 200 mg, orally administered once daily.

Para. IE. The method of any one of Paras. EA-EF, EH-EW, EY, EZ, FA-GA, HA-HF, HH-HW, HY, or HZ, wherein the initial dose is 400 mg, orally administered once daily, and the reduced dose is 300 mg, orally administered once daily.

Para. JA. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose;
monitoring the patient for adverse reaction; and
continuing to administer entrectinib to the patient in the absence of adverse reaction or the absence of at least Grade 3 severity of adverse reaction in the patient or until cancer progression; or
if the patient experiences Grade 3 or Grade 4 severity of adverse reaction, withholding entrectinib and resuming administration of entrectinib at the initial dose or at a reduced dose upon patient recovery to Grade 1 or less severity of adverse reaction or permanently discontinuing administration of entrectinib if adverse reaction does not resolve within 4 weeks or Grade 4 severity adverse reactions recur.

Para. JB. Entrectinib for use in the treatment of cancer or a use of entrectinib in the manufacture of a medicament for the treatment of cancer in a patient in need thereof, wherein the treatment comprises:
assessing left ventricular ejection fraction (LVEF) of the patient prior to administering entrectinib to the patient;
initiating administration of entrectinib to the patient with an initial dose;
monitoring the patient for clinical signs and symptoms of congestive heart failure (CHF), including shortness of breath and edema;
optionally obtaining a MRI or cardiac biopsy to make a diagnosis of congestive heart failure; and
continuing to administer entrectinib to the patient in the absence of adverse reaction or the absence of at least Grade 2 severity of congestive heart failure in the patient or until cancer progression; or
if the patient experiences Grade 2 or 3 severity of congestive heart failure, withholding administration of entrectinib, instituting appropriate medical management, reassessing LVEF and severity of congestive heart failure, and resuming administration of entrectinib at a reduced dose upon patient recovery to less than or equal to Grade 1 severity of congestive heart failure or permanently discontinuing administration of entrectinib if the patient experiences Grade 4 severity of congestive heart failure.

Para. JC. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose;
monitoring the patient for congestive heart failure; and
continuing to administer entrectinib to the patient in the absence of adverse reaction in the patient or until cancer progression; or
if the patient exhibits at least Grade 1 severity of congestive heart failure, withholding administration of entrectinib until patient recovery to less than or equal to Grade 1 severity of congestive heart failure; and resuming administration of entrectinib at a reduced dose; or permanently discontinuing administration of entrectinib to the patient

Para. JD. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose;
monitoring the patient for congestive heart failure; and
continuing to administer entrectinib to the patient in the absence of adverse reaction or the absence of at least Grade 2 severity of congestive heart failure in the patient or until cancer progression; or
if the patient exhibits Grade 2 or Grade 3 severity of congestive heart failure, withholding administration of entrectinib until patient recovery to less than or equal to Grade 1 severity of congestive heart failure; and resuming administration of entrectinib at a reduced dose; or
if the patient exhibits Grade 4 severity of congestive heart failure, withholding administration of entrectinib until patient recovery to less than or equal to Grade 1 severity of congestive heart failure; and resuming administration of entrectinib at a reduced dose or permanently discontinuing administration of entrectinib to the patient.

Para. JE. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose;
monitoring the patient for cardiac disorders; and
continuing to administer entrectinib to the patient in the absence of adverse reaction or until cancer progression; or
if the patient exhibits at least Grade 1 severity of cardiac disorders (e.g., cardiac failure, ventricular extrasystoles, myocarditis, etc.), withholding administration of entrectinib until patient recovery to less than or equal to Grade 1 severity of cardiac disorders; and resuming administration of entrectinib at a reduced dose.

Para. JF. Entrectinib for use in the treatment of cancer or a use of entrectinib in the manufacture of a medicament for the treatment of cancer in a patient in need thereof, wherein the treatment comprises:
prior to administration of entrectinib to the patient,
   advising the patient of a risk of one or more central nervous system (CNS) adverse reactions from the administration of entrectinib; and
   advising the patient not to drive or operate hazardous machinery if the patient experiences one or more central nervous system adverse reactions from the administration of entrectinib;
initiating administration of entrectinib to the patient with an initial dose;
monitoring the patient for one or more central nervous system adverse reactions; and
continuing to administer entrectinib to the patient in the absence of adverse reaction or the absence of at least Grade 2 severity of central nervous system adverse reaction in the patient or until cancer progression; or
if the patient experiences Grade 2 severity of central nervous system adverse reaction, withholding administration of entrectinib and resuming administration of entrectinib at the initial dose or at a reduced dose upon patient recovery to less than or equal to Grade 1 severity of central nervous system adverse reaction; or
if the patient experiences Grade 3 severity of central nervous system adverse reaction, withholding administration of entrectinib and resuming administration of entrectinib at the reduced dose upon patient recovery to less than or equal to Grade 1 severity of central nervous system adverse reaction; or
if the patient experiences Grade 4 severity of central nervous system adverse reaction, permanently discontinuing administration of entrectinib.

Para. JG. Entrectinib for use in the treatment of cancer or a use of entrectinib in the manufacture of a medicament for the treatment of cancer in a patient in need thereof, wherein the treatment comprises:
initiating administration of entrectinib to the patient;
monitoring the patient for one or more signs or symptoms of skeletal fracture;
if the patient exhibits one or more signs or symptoms of skeletal fracture, promptly evaluating the patient and instituting appropriate medical management; and
continuing to administer entrectinib to the patient in the absence of additional adverse reaction in the patient or until cancer progression.

Para. JH. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose;
monitoring the patient with liver tests, including ALT and AST, every 2 weeks during the first month of administration, then monthly thereafter, and as clinically indicated; and
continuing to administer entrectinib to the patient in the absence of adverse reaction or the absence of at least Grade 3 severity of hepatotoxicity in the patient or until cancer progression; or
if the patient experiences Grade 3 severity of hepatotoxicity, withholding administration of entrectinib and resuming administration of entrectinib at the initial dose upon patient recovery within 4 weeks to less than or equal to Grade 1 severity of hepatotoxicity, or resuming administration of entrectinib at a reduced dose for recurrent Grade 3 severity of hepatotoxicity that resolve within 4 weeks; or permanently discontinuing administration of entrectinib if Grade 3 severity of hepatotoxicity does not resolve within 4 weeks; or
if the patient experiences Grade 4 severity of hepatotoxicity, withholding administration of entrectinib and resuming administration of entrectinib at the reduced dose upon patient recovery within 4 weeks to less than or equal to Grade 1 severity of hepatotoxicity, or permanently discontinuing administration of entrectinib if Grade 4 severity of hepatotoxicity does not resolve within 4 weeks or recurs; or
if the patient exhibits ALT or AST greater than 3 times the upper limit of normal (ULN) with concurrent total bilirubin greater than 1.5 times upper limit of normal in the absence of cholestasis or hemolysis, permanently discontinuing administration of entrectinib.

Para. JI. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose;
monitoring the patient for transaminase elevation; and
continuing to administer entrectinib to the patient in the absence of adverse reaction or the absence of at least Grade 3 severity of transaminase elevation in the patient or until cancer progression; or
if the patient exhibits Grade 3 severity of transaminase elevation, withholding administration of entrectinib and resuming administration of entrectinib to the patient without change in dose upon patient recovery within 4 weeks to less than or equal to Grade 1 severity of transaminase elevation, or resuming administration of entrectinib at a reduced dose for recurrent Grade 3 severity of transaminase elevation that resolve within 4 weeks, or permanently discontinuing administration of entrectinib to the patient if Grade 3 severity of transaminase elevation does not resolve within 4 weeks; or
if the patient exhibits Grade 4 severity of transaminase elevation, withholding administration of entrectinib and resuming administration of entrectinib to the patient without change in dose upon patient recovery within 4 weeks to less than or equal to Grade 1 severity of transaminase elevation, or permanently discontinuing administration of entrectinib to the patient if Grade 4 severity of transaminase elevation does not resolve within 4 weeks or recurs; or
if the patient exhibits ALT or AST elevation greater than 3 times the upper limit of normal with total bilirubin elevation being greater than 2 times upper limit of normal in the absence of cholestasis or hemolysis, permanently discontinuing administration of entrectinib to the patient.

Para. JJ. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose;
monitoring the patient for transaminase elevation; and
continuing to administer entrectinib to the patient in the absence of adverse reaction or the absence of at least Grade 3 severity of transaminase elevation in the patient or until cancer progression; or
if the patient exhibits Grade 3 severity of transaminase elevation, withholding administration of entrectinib and resuming administration of entrectinib to the patient without change in dose upon patient recovery within 4 weeks to less than or equal to Grade 1 severity of transaminase elevation, or resuming administration of entrectinib at a reduced dose for recurrent Grade 3 severity of transaminase elevation that resolve within 4 weeks, or permanently discontinuing administration of entrectinib to the patient if Grade 3 severity of transaminase elevation does not resolve within 4 weeks; or
if the patient exhibits Grade 4 severity of transaminase elevation, withholding administration of entrectinib and resuming administration of entrectinib to the patient at a reduced dose upon patient recovery within 4 weeks to less than or equal to Grade 1 severity of transaminase elevation, or permanently discontinuing administration of entrectinib to the patient if Grade 4 severity of transaminase elevation does not resolve within 4 weeks or recurs; or
if the patient exhibits ALT or AST elevation greater than 3 times the upper limit of normal with total bilirubin elevation being greater than 2 times upper limit of normal in the absence of cholestasis or hemolysis, permanently discontinuing administration of entrectinib to the patient.

Para. JK. Entrectinib for use in the treatment of cancer or a use of entrectinib in the manufacture of a medicament for the treatment of cancer in a patient in need thereof, wherein the treatment comprises:
assessing serum uric acid levels in the patient prior to initiating administration of entrectinib;
initiating administration of entrectinib to the patient with an initial dose;
reassessing serum uric acid levels periodically during administration of entrectinib and monitoring the patient for signs and symptoms of hyperuricemia; and
continuing to administer entrectinib to the patient in the absence of adverse reaction in the patient or until cancer progression; or
if the patient is symptomatic of hyperuricemia or experiences Grade 4 hyperuricemia, initiating treatment with urate-lowering medications as clinically indicated, withholding entrectinib and resuming administration of entrectinib at the initial dose or at a reduced dose upon improvement of signs or symptoms of hyperuricemia.

Para. JL. Entrectinib for use in the treatment of cancer or a use of entrectinib in the manufacture of a medicament for the treatment of cancer in a patient in need thereof, wherein the treatment comprises:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc greater than 500 ms, withholding entrectinib until QTc interval recovers to baseline and resuming administration of entrectinib at the initial dose or at a reduced dose; or
if the patient exhibits Torsade de pointes, polymorphic ventricular tachycardia, or signs or symptoms of serious arrhythmia, permanently discontinuing administration of entrectinib.

Para. JM. Entrectinib for use in the treatment of cancer or a use of entrectinib in the manufacture of a medicament for the treatment of cancer in a patient in need thereof, wherein the treatment comprises:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QT interval; and
continuing to administer entrectinib to the patient in the absence of adverse reaction or the absence of at least Grade 2 severity of QT interval prolongation in the patient or until cancer progression; or
if the patient exhibits Grade 2 severity of QT interval prolongation, withholding entrectinib until patient recovery to baseline and resuming administration of entrectinib at the initial dose; or
if the patient exhibits Grade 3 or Grade 4 severity of QT interval prolongation, withholding entrectinib until patient recovery to baseline and resuming administration of entrectinib at a reduced dose; and permanently discontinuing administration of entrectinib if the patient exhibits signs and symptoms of serious arrhythmia.

Para. JN. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose;
continuing to administer entrectinib to the patient in the absence of adverse reaction or the absence of at least Grade 2 severity of QT interval prolongation in the patient or until cancer progression; or
if the patient exhibits Grade 2 severity of QT interval prolongation, withholding entrectinib until patient recovery to less than or equal to Grade 1 severity of QT interval prolongation and resuming administration of entrectinib at the initial dose; or
if the patient exhibits Grade 3 severity of QT interval prolongation, withholding entrectinib until patient recovery to less than or equal to Grade 1 severity of QT interval prolongation and resuming administration of entrectinib at a reduced dose; or withholding entrectinib until patient recovery within seven days to less than or equal to Grade 1 severity of QT interval prolongation and resuming administration of entrectinib at a reduced dose; or permanently discontinuing administration of entrectinib if there is no patient recovery within seven days to less than or equal to Grade 1 severity of QT interval prolongation; or
if the patient exhibits Grade 4 severity of QT interval prolongation, permanently discontinuing administration of entrectinib.

Para. JO. Entrectinib for use in the treatment of cancer or a use of entrectinib in the manufacture of a medicament for the treatment of cancer in a patient in need thereof, wherein the treatment comprises:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or
if the patient exhibits Torsade de pointes, polymorphic ventricular tachycardia, or signs or symptoms of serious arrhythmia, permanently discontinuing administration of entrectinib.

Para. JP. Entrectinib for use in the treatment of cancer or a use of entrectinib in the manufacture of a medicament for the treatment of cancer in a patient in need thereof, wherein the treatment comprises:
assessing QT interval and electrolytes in the patient prior to initiating administration of entrectinib;
initiating administration of entrectinib to the patient with an initial dose;
reassessing QT interval and electrolytes periodically during administration of entrectinib, adjusting frequency based upon patient's risk factors such as congestive heart failure, electrolyte abnormalities, or concomitant medications known to prolong the QTc interval; and
continuing to administer entrectinib to the patient in the absence of adverse reaction in the patient or until cancer progression; or
if the patient exhibits QTc of 481 to 500 ms, withholding entrectinib until QTc interval recovers to baseline and resuming administration of entrectinib at the initial dose; or
if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or
if the patient exhibits Torsade de pointes, polymorphic ventricular tachycardia, or signs or symptoms of serious arrhythmia, permanently discontinuing administration of entrectinib.

Para. JQ. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose;
monitoring the patient for a new vision disorder; and
continuing to administer entrectinib to the patient in the absence of adverse reaction or the absence of at least Grade 2 severity of vision disorder in the patient or until cancer progression; or
if the patient experiences Grade 2 or higher severity of vision disorder, withholding entrectinib until improvement or stabilization of the vision disorder, and resuming administration of entrectinib at the initial dose or at a reduced dose.

Para. JR. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose;
monitoring the patient for anemia or neutropenia; and
continuing to administer entrectinib to the patient in the absence of adverse reaction or the absence of at least Grade 3 severity of anemia or neutropenia in the patient or until cancer progression; or
if the patient experiences Grade 3 or Grade 4 severity of anemia or neutropenia, withholding entrectinib and resuming administration of entrectinib at the initial dose or at a reduced dose upon patient recovery to Grade 2 or less severity of anemia or neutropenia.

Para JS. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose;
monitoring the patient for anemia or neutropenia; and
continuing to administer entrectinib to the patient in the absence of adverse reaction or the absence of at least Grade 3 severity of anemia or neutropenia in the patient or until cancer progression; or
if the patient experiences Grade 3 severity of anemia or neutropenia, withholding entrectinib and resuming administration of entrectinib at the initial dose or at a reduced dose upon patient recovery to Grade 2 or less severity of anemia or neutropenia; or
if the patient experiences Grade 4 severity of anemia or neutropenia, withholding entrectinib and resuming administration of entrectinib at a reduced dose upon patient recovery to Grade 2 or less severity of anemia or neutropenia.

Para. JT. Entrectinib for use in the treatment of cancer or a use of entrectinib in the manufacture of a medicament for the treatment of cancer in a patient in need thereof, wherein the treatment comprises:
prior to the administration of entrectinib to the patient,
   advising the patient of a potential for cognitive change from the administration of entrectinib; and
   advising the patient not to drive or operate hazardous machinery if the patient experiences one or more symptoms of cognitive change from the administration of entrectinib;
initiating administration of entrectinib to the patient with an initial dose;
monitoring the patient for a cognitive disorder; and
continuing to administer entrectinib to the patient in the absence of adverse reaction or the absence of at least Grade 2 severity of cognitive disorder in the patient or until cancer progression; or
if the patient experiences an event of at least Grade 2 severity of cognitive disorder, withholding administration of entrectinib until patient recovery to less than or equal to Grade 1 severity of cognitive disorder and resuming administration of entrectinib at a reduced dose; or
if the patient experiences a recurrence of the event of at least Grade 2 severity of cognitive disorder, administering entrectinib at a further reduced dose; or
if the patient experiences prolonged, severe, or intolerable events of cognitive disorder, permanently discontinuing administration of entrectinib.

Para. JU. Entrectinib for use or the use of Para. JT, wherein the cognitive change or cognitive disorder comprises one or more of confusion, mental status changes, memory impairment, and hallucinations.

Para. JV. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose;
monitoring the patient for a cognitive disorder (e.g., confusion, mental status changes, memory impairment, hallucinations, dysarthria, etc.) or ataxia; and
continuing to administer entrectinib to the patient in the absence of adverse reaction or the absence of at least Grade 2 severity of cognitive disorder or ataxia in the patient or until cancer progression; or
if the patient experiences a first event of at least Grade 2 severity of cognitive disorder or ataxia, withholding administration of entrectinib until patient recovery to less than or equal to Grade 1 severity of cognitive disorder or ataxia and resuming administration of entrectinib at a reduced dose; or
if the patient experiences a recurrence of the event of at least Grade 2 severity of cognitive disorder or ataxia, administering entrectinib at a further reduced dose; or permanently discontinuing administration of entrectinib.

Para. JW. Entrectinib for use in the treatment of cancer or a use of entrectinib in the manufacture of a medicament for the treatment of cancer in a patient in need thereof, wherein the treatment comprises:
prior to the administration of entrectinib to the patient,
   advising the patient of a potential for cognitive change from the administration of entrectinib; and
   advising the patient not to drive or operate hazardous machinery if the patient experiences one or more symptoms of cognitive change from the administration of entrectinib;
initiating administration of entrectinib to the patient with an initial dose;
monitoring the patient for a cognitive disorder; and
continuing to administer entrectinib to the patient in the absence of adverse reaction or the absence of at least Grade 2 severity of cognitive disorder in the patient or until cancer progression; or
if the patient experiences Grade 2 severity of cognitive disorder, withholding administration of entrectinib until patient recovery to less than or equal to Grade 1 severity of cognitive disorder and resuming administration of entrectinib at the initial dose or a reduced dose; or
if the patient experiences Grade 3 severity of cognitive disorder, withholding administration of entrectinib until patient recovery to less than or equal to Grade 1 severity of cognitive disorder and resuming administration of entrectinib at a reduced dose; or
if the patient experiences prolonged, severe, or intolerable events of cognitive disorder, permanently discontinuing administration of entrectinib.

Para. JX. Entrectinib for use in the treatment of cancer or a use of entrectinib in the manufacture of a medicament for the treatment of cancer in a patient in need thereof, wherein the treatment comprises:
prior to the administration of entrectinib to the patient,
   advising the patient of a potential for embryo-fetal toxicity;
initiating administration of entrectinib to the patient; and
continuing to administer entrectinib to the patient in the absence of adverse reaction in the patient or until cancer progression; or
if the patient becomes pregnant, permanently discontinuing administration of entrectinib.

Para. JY. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose; and
monitoring the patient for adverse reaction; and
continuing to administer entrectinib to the patient in the absence of syncope in the patient or until cancer progression; or
if the patient experiences a first event of at least Grade 1 severity of syncope, withholding administration of entrectinib until patient recovery to baseline and resuming administration of entrectinib at a reduced dose; or
if the patient experiences a recurrence of at least Grade 1 severity of syncope, administering entrectinib at a further reduced dose; or permanently discontinuing administration of entrectinib.

Para. JZ. Entrectinib for use in the treatment of cancer or a use of entrectinib in the manufacture of a medicament for the treatment of cancer in a patient in need thereof, wherein the treatment comprises:
initiating administration of entrectinib to the patient with an initial dose; and
monitoring the patient for interstitial lung disease; and
continuing to administer entrectinib to the patient in the absence of interstitial lung disease in the patient or until cancer progression; or
if the patient experiences a first event of Grade 1 or Grade 2 severity of interstitial lung disease, withholding administration of entrectinib until patient recovery to baseline and resuming administration of entrectinib at the initial dose; or
if the patient experiences a recurrence of Grade 1 or Grade 2 severity of interstitial lung disease or the patient experiences Grade 3 or Grade 4 severity of interstitial lung disease, permanently discontinuing administration of entrectinib.

Para. KA. Entrectinib for use in the treatment of cancer or a use of entrectinib in the manufacture of a medicament for the treatment of cancer in a patient in need thereof, wherein the treatment comprises:
prior to administering entrectinib to the patient,
   (i) optionally advising the patient of a risk of one or more adverse reactions from the administration of entrectinib; and/or
   (ii) optionally assessing one or more parameters in the patient associated with an adverse reaction (e.g., LVEF for CHF, serum uric acid levels for hyperuricemia, QT interval and electrolytes for QT interval prolongation);
initiating administration of entrectinib to the patient with an initial dose (e.g., 600 mg orally per day);
monitoring the patient for adverse reaction; and
if the patient does not exhibit a severe adverse reaction, continuing to administer entrectinib until cancer progression; or
if the patient experiences a severe adverse reaction, withholding entrectinib and, upon patient recovery, resuming administration of entrectinib at the initial dose or at a reduced dose wherein patient recovery is either the absence of or a reduction of the severe adverse reaction or if the severe adverse reaction does not resolve within 4 weeks or if the severe adverse reaction recurs, permanently discontinuing administration of entrectinib.

Para. KB. Entrectinib for use or the use of Para. KA, wherein the adverse reaction is clinical signs and symptoms of congestive heart failure, including shortness of breath and edema; and the severe adverse reaction is at least Grade 2 severity of congestive heart failure; wherein the one or more parameters comprise left ventricular ejection fraction (LVEF); wherein the treatment further comprises optionally obtaining a MRI or cardiac biopsy to make a diagnosis of congestive heart failure; and wherein if the patient experiences Grade 2 or 3 severity of congestive heart failure, the treatment comprises withholding administration of entrectinib, instituting appropriate medical management, reassessing LVEF and severity of congestive heart failure, and resuming administration of entrectinib at a reduced dose upon patient recovery or permanently discontinuing administration of entrectinib if the patient experiences Grade 4 severity of congestive heart failure.

Para. KC. Entrectinib for use or the use of Para. KA, wherein the adverse reaction is congestive heart failure; and the severe adverse reaction is at least Grade 1 severity of congestive heart failure, wherein patient recovery is determined as less than or equal to a Grade 1 severity of congestive heart failure; and wherein if the patient exhibits at least Grade 1 severity of congestive heart failure, the treatment comprises withholding administration of entrectinib until patient recovery; and resuming administration of entrectinib at a reduced dose or permanently discontinuing administration of entrectinib to the patient.

Para. KD. Entrectinib for use or the use of Para. KA, wherein the adverse reaction is congestive heart failure; and the severe adverse reaction is at least Grade 2 severity of congestive heart failure, and wherein patient recovery is determined as less than or equal to a Grade 1 severity of congestive heart failure; wherein if the patient exhibits at least Grade 2 severity of congestive heart failure, the treatment comprises withholding administration of entrectinib until patient recovery; and resuming administration of entrectinib at a reduced dose or permanently discontinuing administration of entrectinib to the patient.

Para. KE. Entrectinib for use or the use of Para. KA, wherein the adverse reaction is cardiac disorders (e.g., cardiac failure, ventricular extrasystoles, myocarditis, etc.); and the severe adverse reaction is at least Grade 1 severity of cardiac disorders, wherein patient recovery is determined as less than or equal to a Grade 1 severity of cardiac disorders; and wherein if the patient exhibits at least Grade 1 severity of cardiac disorders, the treatment comprises withholding administration of entrectinib until patient recovery; and resuming administration of entrectinib at a reduced dose or permanently discontinuing administration of entrectinib to the patient.

Para. KF. Entrectinib for use or the use of Para. KA, wherein the adverse reaction is a central nervous system (CNS) adverse reaction; and the severe adverse reaction is at least Grade 2 severity of central nervous system adverse reaction; and wherein patient recovery is determined as less than or equal to a Grade 1 severity of central nervous system adverse reaction; wherein prior to administration of entrectinib to the patient, the treatment further comprises advising the patient not to drive or operate hazardous machinery if the patient experiences the one or more central nervous system adverse reactions from the administration of entrectinib; and wherein if the patient exhibits a Grade 2 severity of central nervous system adverse reaction, the treatment comprises withholding administration of entrectinib and resuming administration of entrectinib at the initial dose or at a reduced dose upon patient recovery; or if the patient experiences Grade 3 severity of central nervous system adverse reaction, withholding administration of entrectinib and resuming administration of entrectinib at the reduced dose upon patient recovery; or if the patient experiences Grade 4 severity of central nervous system adverse reaction, permanently discontinuing administration of entrectinib.

Para. KG. Entrectinib for use or the use of Para. KA, wherein the adverse reaction is skeletal fracture; and wherein if the patient exhibits one or more signs or symptoms of skeletal fracture, the treatment further comprises promptly evaluating the patient and instituting appropriate medical management.

Para. KH. Entrectinib for use or the use of Para. KA, wherein the adverse reaction is hepatotoxicity; the monitoring comprises liver tests, including measurement of ALT and AST, every 2 weeks during the first month of administration, then monthly thereafter, and as clinically indicated; wherein the severe adverse reaction is at least a Grade 3 severity of hepatotoxicity; wherein patient recovery is determined as less than or equal to a Grade 1 severity of hepatotoxicity; and wherein if the patient experiences Grade 3 severity of hepatotoxicity, the treatment comprises withholding administration of entrectinib and resuming administration of entrectinib at the initial dose upon patient recovery within 4 weeks, or resuming administration of entrectinib at a reduced dose for recurrent Grade 3 severity of hepatotoxicity that resolve within 4 weeks; or permanently discontinuing administration of entrectinib if Grade 3 severity of hepatotoxicity does not resolve within 4 weeks; or if the patient experiences Grade 4 severity of hepatotoxicity, withholding administration of entrectinib and resuming administration of entrectinib at the reduced dose upon patient recovery within 4 weeks, or permanently discontinuing administration of entrectinib if Grade 4 severity of hepatotoxicity does not resolve within 4 weeks or recurs; or if the patient exhibits ALT or AST greater than 3 times the upper limit of normal (ULN) with concurrent total bilirubin greater than 1.5 times upper limit of normal in the absence of cholestasis or hemolysis, permanently discontinuing administration of entrectinib.

Para. KI. Entrectinib for use or the use of Para. KA, wherein the adverse reaction is transaminase elevation; and the severe adverse reaction is at least a Grade 3 severity of transaminase elevation; wherein patient recovery is determined as less than or equal to a Grade 1 severity of transaminase elevation; and wherein if the patient exhibits Grade 3 severity of transaminase elevation, the treatment comprises withholding administration of entrectinib and resuming administration of entrectinib to the patient without change in dose upon patient recovery within 4 weeks, or resuming administration of entrectinib at a reduced dose for recurrent Grade 3 severity of transaminase elevation that resolve within 4 weeks, or permanently discontinuing administration of entrectinib to the patient if Grade 3 severity of transaminase elevation does not resolve within 4 weeks; or if the patient exhibits Grade 4 severity of transaminase elevation, withholding administration of entrectinib and resuming administration of entrectinib to the patient without change in dose upon patient recovery within 4 weeks, or permanently discontinuing administration of entrectinib to the patient if Grade 4 severity of transaminase elevation does not resolve within 4 weeks or recurs; or if the patient exhibits ALT or AST elevation greater than 3 times the upper limit of normal with total bilirubin elevation being greater than 2 times upper limit of normal in the absence of cholestasis or hemolysis, permanently discontinuing administration of entrectinib to the patient.

Para. KJ. Entrectinib for use or the use of Para. KA, wherein the adverse reaction is transaminase elevation; and the severe adverse reaction is at least a Grade 3 severity of transaminase elevation; wherein patient recovery is determined as less than or equal to a Grade 1 severity of transaminase elevation; and wherein if the patient exhibits Grade 3 severity of transaminase elevation, the treatment comprises withholding administration of entrectinib and resuming administration of entrectinib to the patient without change in dose upon patient recovery within 4 weeks, or resuming administration of entrectinib at a reduced dose for recurrent Grade 3 severity of transaminase elevation that resolve within 4 weeks, or permanently discontinuing administration of entrectinib to the patient if Grade 3 severity of transaminase elevation does not resolve within 4 weeks; or if the patient exhibits Grade 4 severity of transaminase elevation, withholding administration of entrectinib and resuming administration of entrectinib to the patient at a reduced dose upon patient recovery within 4 weeks, or permanently discontinuing administration of entrectinib to the patient if Grade 4 severity of transaminase elevation does not resolve within 4 weeks or recurs; or if the patient exhibits ALT or AST elevation greater than 3 times the upper limit of normal with total bilirubin elevation being greater than 2 times upper limit of normal in the absence of cholestasis or hemolysis, permanently discontinuing administration of entrectinib to the patient.

Para. KK. Entrectinib for use or the use of Para. KA, wherein the adverse reaction is hyperuricemia; wherein the one or more parameters comprise serum uric acid levels in the patient; wherein the treatment further comprises reassessing serum uric acid levels periodically during administration of entrectinib; wherein the severe adverse reaction is symptoms of hyperuricemia or Grade 4 hyperuricemia; and wherein if the patient is symptomatic of hyperuricemia or experiences Grade 4 hyperuricemia, the treatment comprises initiating treatment with urate-lowering medications as clinically indicated, withholding entrectinib and resuming administration of entrectinib at the initial dose or at a reduced dose upon improvement of signs or symptoms of hyperuricemia.

Para. KL. Entrectinib for use or the use of Para. KA, wherein the adverse reaction is prolongation of the QTc interval; wherein the one or more parameters comprise QT interval and electrolytes in the patient; wherein the treatment further comprises reassessing QT interval and electrolytes periodically during administration of entrectinib, adjusting frequency based upon patient's risk factors such as congestive heart failure, electrolyte abnormalities, or concomitant medications known to prolong the QTc interval; and wherein if the patient exhibits QTc greater than 500 ms, the treatment further comprises withholding entrectinib until QTc interval recovers to baseline and resuming administration of entrectinib at the initial dose or at a reduced dose; or if the patient exhibits Torsade de pointes, polymorphic ventricular tachycardia, or signs or symptoms of serious arrhythmia, permanently discontinuing administration of entrectinib.

Para. KM. Entrectinib for use or the use of Para. KA, wherein the adverse reaction is QT interval prolongation; and the severe adverse reaction is at least a Grade 2 severity of QT interval prolongation; wherein the one or more parameters comprise QT interval and electrolytes in the patient; wherein the treatment further comprises reassessing QT interval and electrolytes periodically during administration of entrectinib, adjusting frequency based upon patient's risk factors such as congestive heart failure, electrolyte abnormalities, or concomitant medications known to prolong the QT interval; and wherein if the patient exhibits Grade 2 severity of QT interval prolongation, the treatment comprises withholding entrectinib until patient recovery to baseline and resuming administration of entrectinib at the initial dose; or if the patient exhibits Grade 3 or Grade 4 severity of QT interval prolongation, withholding entrectinib until patient recovery to baseline and resuming administration of entrectinib at a reduced dose; and permanently discontinuing administration of entrectinib if the patient exhibits signs and symptoms of serious arrhythmia.

Para. KN. Entrectinib for use or the use of Para. KA, wherein the adverse reaction is QT interval prolongation; and the severe adverse reaction is at least a Grade 2 severity of QT interval prolongation; wherein patient recovery is determined as less than or equal to a Grade 1 severity of QT interval prolongation; and wherein if the patient exhibits Grade 2 severity of QT interval prolongation, the treatment comprises withholding entrectinib until patient recovery and resuming administration of entrectinib at the initial dose; or if the patient exhibits Grade 3 severity of QT interval prolongation, withholding entrectinib until patient recovery and resuming administration of entrectinib at a reduced dose; or withholding entrectinib until patient recovery within seven days and resuming administration of entrectinib at a reduced dose; or permanently discontinuing administration of entrectinib if there is no patient recovery within seven days; or if the patient exhibits Grade 4 severity of QT interval prolongation, permanently discontinuing administration of entrectinib.

Para. KO. Entrectinib for use or the use of Para. KA, wherein the adverse reaction is prolongation of the QTc interval; wherein the one or more parameters comprise QT interval and electrolytes in the patient; wherein the treatment further comprises reassessing QT interval and electrolytes periodically during administration of entrectinib, adjusting frequency based upon patient's risk factors such as congestive heart failure, electrolyte abnormalities, or concomitant medications known to prolong the QTc interval; and wherein if the patient exhibits QTc greater than 500 ms, the treatment comprises withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or if the patient exhibits Torsade de pointes, polymorphic ventricular tachycardia, or signs or symptoms of serious arrhythmia, permanently discontinuing administration of entrectinib.

Para. KP. Entrectinib for use or the use of Para. KA, wherein the adverse reaction is prolongation of the QTc interval; wherein the one or more parameters comprise QT interval and electrolytes in the patient; wherein the treatment further comprises reassessing QT interval and electrolytes periodically during administration of entrectinib, adjusting frequency based upon patient's risk factors such as congestive heart failure, electrolyte abnormalities, or concomitant medications known to prolong the QTc interval; and wherein if the patient exhibits QTc of 481 to 500 ms, the treatment comprises withholding entrectinib until QTc interval recovers to baseline and resuming administration of entrectinib at the initial dose; or if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or if the patient exhibits Torsade de pointes, polymorphic ventricular tachycardia, or signs or symptoms of serious arrhythmia, permanently discontinuing administration of entrectinib.

Para. KQ. Entrectinib for use or the use of Para. KA, wherein the adverse reaction is a vision disorder; and the severe adverse reaction is at least a Grade 2 severity of vision disorder; and wherein if the patient experiences Grade 2 or higher severity of vision disorder, the treatment comprises withholding entrectinib until improvement or stabilization of the vision disorder, and resuming administration of entrectinib at the initial dose or at a reduced dose.

Para. KR. Entrectinib for use or the use of Para. KA, wherein the adverse reaction is anemia or neutropenia; and the severe adverse reaction is at least a Grade 3 severity of anemia or neutropenia; and wherein if the patient experiences Grade 3 or Grade 4 severity of anemia or neutropenia, the treatment comprises withholding entrectinib and resuming administration of entrectinib at the initial dose or at a reduced dose upon patient recovery to Grade 2 or less severity of anemia or neutropenia.

Para. KS. Entrectinib for use or the use of Para. KA, wherein the adverse reaction is anemia or neutropenia; and the severe adverse reaction is at least a Grade 3 severity of anemia or neutropenia; wherein patient recovery is determined as less than or equal to a Grade 2 severity of anemia or neutropenia; and wherein if the patient experiences Grade 3 severity of anemia or neutropenia, the treatment comprises withholding entrectinib and resuming administration of entrectinib at the initial dose or at a reduced dose upon patient recovery; or if the patient experiences Grade 4 severity of anemia or neutropenia, withholding entrectinib and resuming administration of entrectinib at a reduced dose upon patient recovery.

Para. KT. Entrectinib for use or the use of Para. KA, wherein the adverse reaction is a cognitive disorder; and the severe adverse reaction is at least a Grade 2 severity of cognitive disorder; wherein patient recovery is determined as less than or equal to a Grade 1 severity of cognitive disorder; wherein prior to administration of entrectinib to the patient, the treatment further comprises advising the patient of a potential for cognitive change from the administration of entrectinib and advising the patient not to drive or operate hazardous machinery if the patient experiences one or more symptoms of cognitive change from the administration of entrectinib; and wherein if the patient experiences an event of at least Grade 2 severity of cognitive disorder, the treatment comprises withholding administration of entrectinib until patient recovery and resuming administration of entrectinib at a reduced dose; or if the patient experiences a recurrence of the event of at least Grade 2 severity of cognitive disorder, administering entrectinib at a further reduced dose; or if the patient experiences prolonged, severe, or intolerable events of cognitive disorder, permanently discontinuing administration of entrectinib.

Para. KU. Entrectinib for use or the use of Para. KT, wherein cognitive change or cognitive disorder comprises one or more of confusion, mental status changes, memory impairment, and hallucinations.

Para. KV. Entrectinib for use or the use of Para. KA, wherein the adverse reaction is a cognitive disorder or ataxia; and the severe adverse reaction is at least a Grade 2 severity of cognitive disorder or ataxia; wherein patient recovery is determined as less than or equal to a Grade 1 severity of cognitive disorder or ataxia; and wherein if the patient experiences a first event of at least Grade 2 severity of cognitive disorder or ataxia, the treatment comprises withholding administration of entrectinib until patient recovery and resuming administration of entrectinib at a reduced dose; or if the patient experiences a recurrence of the event of at least Grade 2 severity of cognitive disorder or ataxia, administering entrectinib at a further reduced dose; or permanently discontinuing administration of entrectinib.

Para. KW. Entrectinib for use or the use of Para. KA, wherein the adverse reaction is a cognitive disorder; and the severe adverse reaction is at least a Grade 2 severity of cognitive disorder; wherein patient recovery is determined as less than or equal to a Grade 1 severity of cognitive disorder; wherein prior to administration of entrectinib to the patient, the treatment further comprises advising the patient of a potential for cognitive change from the administration of entrectinib and advising the patient not to drive or operate hazardous machinery if the patient experiences one or more symptoms of cognitive change from the administration of entrectinib; and wherein if the patient experiences an event of at least Grade 2 severity of cognitive disorder, the treatment comprises withholding administration of entrectinib until patient recovery and resuming administration of entrectinib at the initial dose or a reduced dose; or if the patient experiences Grade 3 severity of cognitive disorder, withholding administration of entrectinib until patient recovery and resuming administration of entrectinib at a reduced dose; or if the patient experiences prolonged, severe, or intolerable events of cognitive disorder, permanently discontinuing administration of entrectinib.

Para. KX. Entrectinib for use or the use of Para. KA, wherein the adverse reaction is embryo-fetal toxicity; and wherein if the patient becomes pregnant, the treatment further comprises permanently discontinuing administration of entrectinib.

Para. KY. Entrectinib for use or the use of Para. KA, wherein the adverse reaction is syncope; the severe adverse reaction is at least a Grade 1 severity of syncope; wherein patient recovery is determined as recovery to baseline; and wherein if the patient experiences a first event of at least Grade 1 severity of syncope, the treatment comprises withholding administration of entrectinib until patient recovery and resuming administration of entrectinib at a reduced dose; or if the patient experiences a recurrence of at least Grade 1 severity of syncope, administering entrectinib at a further reduced dose; or permanently discontinuing administration of entrectinib.

Para. KZ. Entrectinib for use or the use of Para. KA, wherein the adverse reaction is interstitial lung disease; the severe adverse reaction is at least a Grade 1 severity of interstitial lung disease; wherein patient recovery is determined as recovery to baseline; and wherein if the patient experiences a first event of Grade 1 or Grade 2 severity of syncope, the treatment comprises withholding administration of entrectinib until patient recovery and resuming administration of entrectinib at the initial dose; or if the patient experiences a recurrence of Grade 1 or Grade 2 severity of interstitial lung disease or the patient experiences Grade 3 or Grade 4 severity of interstitial lung disease, permanently discontinuing administration of entrectinib.

Para. LA. Entrectinib for use or the use of Para. KA, wherein the adverse reaction is congestive heart failure, a central nervous system (CNS) adverse reaction, fracture (e.g., skeletal fracture), hepatotoxicity, hyperuricemia, QT interval prolongation, a vision disorder, anemia/neutropenia, a cognitive disorder, transaminase elevation, or an embryo-fetal toxicity.

Para. LB. Entrectinib for use or the use of Paras. JA-JF, JH-JW, JY, JZ, or KA-LA, wherein the initial dose is 600 mg, orally administered once daily, and the reduced dose is 400 mg, orally administered once daily.

Para. LC. Entrectinib for use or the use of Paras. JA-JF, JH-JW, JY, JZ, or KA-LA, wherein the initial dose is 100 mg, orally administered once daily, and the reduced dose is 100 mg, orally administered once per day for 5 days each week.

Para. LD. Entrectinib for use or the use of Paras. JA-JF, JH-JW, JY, JZ, or KA-LA, wherein the initial dose is 200 mg, orally administered once daily, and the reduced dose is 200 mg, orally administered once per day for 5 days each week.

Para. LE. Entrectinib for use or the use of Paras. JA-JF, JH-JW, JY, JZ, or KA-LA, wherein the initial dose is 300 mg, orally administered once daily, and the reduced dose is 200 mg, orally administered once daily.

Para. LF. Entrectinib for use or the use of Paras. JA-JF, JH-JW, JY, JZ, or KA-LA, wherein the initial dose is 400 mg, orally administered once daily, and the reduced dose is 300 mg, orally administered once daily.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A method of treating cancer in a patient in need thereof, the method comprising administering to the patient a therapeutically effective amount of entrectinib wherein the patient has impaired hepatic function.

2. The method of claim 1, wherein the impaired hepatic function is mild, moderate, or severe hepatic dysfunction, as assessed by Child-Pugh scoring system.

3.-7. (canceled)

8. The method of claim 1, wherein the impaired hepatic function is mild, moderate, or severe hepatic impairment as assessed by National Cancer Institute organ dysfunction working group system.

9. (canceled)

10. The method of claim 1, wherein the patient has a total bilirubin concentration that is about greater than 1.5 times the upper limit of normal to about 3 times the upper limit of normal.

11. (canceled)

12. The method of claim 1, wherein the therapeutically effective amount is 600 mg or less.

13. (canceled)

14. The method of claim 1, wherein entrectinib is administered once or twice daily.

15. (canceled)

16. The method of claim 1, wherein the patient has at least one genetic alteration in at least one target gene selected from ROS1, NTRK1, NTRK2, and NTRK3.

17. (canceled)

18. (canceled)

19. The method of claim 1, wherein the cancer is selected from sarcoma, non-small cell lung cancer, mammary analogue secretory carcinoma, breast cancer, thyroid cancer, colorectal cancer, neuroendocrine cancers, pancreatic cancer, gynecological cancers, and cholangiocarcinoma.

20. The method of claim 1, wherein the cancer comprises one or more NTRK gene fusion-positive solid tumors, or the cancer is ROS-1 positive non-small cell lung cancer.

21.-23. (canceled)

24. A method of treating cancer or managing adverse reactions of administration of entrectinib in a patient in need thereof, the method comprising:
prior to administering entrectinib to the patient,
(i) optionally advising the patient of a risk of one or more adverse reactions from the administration of entrectinib; and/or
(ii) optionally assessing one or more parameters in the patient associated with an adverse reaction;
initiating administration of entrectinib to the patient with an initial dose;
monitoring the patient for adverse reaction; and
if the patient does not exhibit a severe adverse reaction, continuing to administer entrectinib until cancer progression; or
if the patient experiences a severe adverse reaction, withholding entrectinib and, upon patient recovery, resuming administration of entrectinib at the initial dose or at a reduced dose wherein patient recovery is either the absence of or a reduction of the severe adverse reaction or if the severe adverse reaction does not resolve within 4 weeks or if the severe adverse reaction recurs, permanently discontinuing administration of entrectinib.

25. The method of claim 24, wherein the adverse reaction is congestive heart failure, a cardiac disorder, a central nervous system (CNS) adverse reaction, fracture, hepatotoxicity, hyperuricemia, a vision disorder, anemia/neutropenia, a cognitive disorder, transaminase elevation, an embryo-fetal toxicity, syncope, or an interstitial lung disease.

26. The method of claim 24, wherein the adverse reaction is congestive heart failure; and the severe adverse reaction is at least Grade 2 severity of congestive heart failure, and wherein patient recovery is determined as less than or equal to a Grade 1 severity of congestive heart failure; wherein if the patient exhibits Grade 4 severity of congestive heart failure, the method comprises withholding administration of entrectinib until patient recovery to less than or equal to Grade 1 severity of congestive heart failure; and resuming administration of entrectinib at a reduced dose or permanently discontinuing administration of entrectinib to the patient; or
wherein the adverse reaction is prolongation of the QTc interval; wherein the one or more parameters comprise QT interval and electrolytes in the patient wherein the method comprises reassessing QT interval and electrolytes periodically during administration of entrectinib, adjusting frequency based upon patient's risk factors such as congestive heart failure, electrolyte abnormalities, or concomitant medications known to prolong the QTc interval; and wherein if the patient exhibits QTc of 481 to 500 ms, the method further comprises withholding entrectinib until QTc interval recovers to baseline and resuming administration of entrectinib at the initial dose; or if the patient exhibits QTc greater than 500 ms, withholding entrectinib until QTc interval recovers to baseline; and resuming administration of entrectinib at the initial dose if factors that cause QT prolongation are identified and corrected, or resuming administration of entrectinib at a reduced dose if other factors that cause QT prolongation are not identified; or if the patient exhibits Torsade de pointes, polymorphic ventricular tachycardia, or signs or symptoms of serious arrhythmia, permanently discontinuing administration of entrectinib.

27. The method of claim 24, wherein the adverse reaction is a central nervous system (CNS) adverse reaction; and the severe adverse reaction is at least a Grade 2 severity of central nervous system adverse reaction; wherein patient recovery is determined as less than or equal to a Grade 1 severity of central nervous system adverse reaction; wherein prior to administration of entrectinib to the patient, the method further comprises advising the patient not to drive or operate hazardous machinery if the patient experiences the one or more central nervous system adverse reactions from the administration of entrectinib; and wherein if the patient exhibits a Grade 2 severity of central nervous system adverse reaction, the method comprises withholding administration of entrectinib and resuming administration of entrectinib at the initial dose or at a reduced dose upon patient recovery; or if the patient experiences Grade 3 severity of central nervous system adverse reaction, withholding administration of entrectinib and resuming administration of entrectinib at the reduced dose upon patient; or if the patient experiences Grade 4 severity of central nervous system adverse reaction, permanently discontinuing administration of entrectinib; or
wherein the adverse reaction is a cognitive disorder; and the severe adverse reaction is at least a Grade 2 severity of cognitive disorder; wherein prior to administration of entrectinib to the patient, the method further comprises advising the patient of a potential for cognitive change from the administration of entrectinib and advising the patient not to drive or operate hazardous machinery if the patient experiences one or more symptoms of cognitive change from the administration of entrectinib; and wherein if the patient experiences an event of at least Grade 2 severity of cognitive disorder, the method comprises withholding administration of entrectinib until patient recovery to less than or equal to Grade 1 severity of cognitive disorder and resuming administration of entrectinib at a reduced dose; or if the patient experiences a recurrence of the event of at least Grade 2 severity of cognitive disorder, administering entrectinib at a further reduced dose; or if the patient experiences prolonged, severe, or intolerable events of cognitive disorder, permanently discontinuing administration of entrectinib.

28. (canceled)

29. (canceled)

30. The method of claim 24, wherein the adverse reaction is skeletal fracture; and wherein if the patient exhibits one or more signs or symptoms of skeletal fracture, the method further comprises promptly evaluating the patient and instituting appropriate medical management.

31. The method of claim 24, wherein the adverse reaction is hepatotoxicity; the monitoring comprises liver tests, including measurement of ALT and AST, every 2 weeks during the first month of administration, then monthly thereafter, and as clinically indicated; wherein the severe adverse reaction is at least a Grade 3 severity of hepatotoxicity;
wherein patient recovery is determined as less than or equal to a Grade 1 severity of hepatotoxicity; and wherein if the patient experiences Grade 3 severity of hepatotoxicity, the method comprises withholding administration of entrectinib and resuming administration of entrectinib at the initial dose upon patient recovery within 4 weeks, or resuming administration of entrectinib at a reduced dose for recurrent Grade 3 severity of hepatotoxicity that resolve within 4 weeks; or permanently discontinuing administration of entrectinib if Grade 3 severity of hepatotoxicity does not resolve within 4 weeks; or if the patient experiences Grade 4 severity of hepatotoxicity, withholding administration of entrectinib and resuming administration of entrectinib at the reduced dose upon patient recovery within 4 weeks, or permanently discontinuing administration of entrectinib if Grade 4 severity of hepatotoxicity does not resolve within 4 weeks or recurs; or if the patient exhibits ALT or AST greater than 3 times the upper limit of normal (ULN) with concurrent total bilirubin greater than 1.5 times upper limit of normal in the absence of cholestasis or hemolysis, permanently discontinuing administration of entrectinib; or
wherein the adverse reaction is transaminase elevation; and the severe adverse reaction is at least a Grade 3 severity of transaminase elevation; wherein patient recovery is determined as less than or equal to a Grade 1 severity of transaminase elevation; and wherein if the patient exhibits Grade 3 severity of transaminase elevation, the method comprises withholding administration of entrectinib and resuming administration of entrectinib to the patient without change in dose upon patient recovery within 4 weeks, or resuming administration of entrectinib at a reduced dose for recurrent Grade 3 severity of transaminase elevation that resolve within 4 weeks, or permanently discontinuing administration of entrectinib to the patient if Grade 3 severity of transaminase elevation does not resolve within 4 weeks; or if the patient exhibits Grade 4 severity of transaminase elevation, withholding administration of entrectinib and resuming administration of entrectinib to the patient without change in dose upon patient recovery within 4 weeks, or permanently discontinuing administration of entrectinib to the patient if Grade 4 severity of transaminase elevation does not resolve within 4 weeks or recurs; or if the patient exhibits ALT or AST elevation greater than 3 times the upper limit of normal with total bilirubin elevation being greater than 2 times upper limit of normal in the absence of cholestasis or hemolysis, permanently discontinuing administration of entrectinib to the patient; or
wherein the adverse reaction is hyperuricemia; wherein the one or more parameters comprise serum uric acid levels in the patient wherein the method further comprises reassessing serum uric acid levels periodically during administration of entrectinib; wherein the severe adverse reaction is symptoms of hyperuricemia or Grade 4 hyperuricemia; and wherein if the patient is symptomatic of hyperuricemia or experiences Grade 4 hyperuricemia, the method comprises initiating treatment with urate-lowering medications as clinically indicated, withholding entrectinib and resuming administration of entrectinib at the initial dose or at a reduced dose upon improvement of signs or symptoms of hyperuricemia.

32.-34. (canceled)

35. The method of claim 24, wherein the adverse reaction is a vision disorder; and the severe adverse reaction is at least a Grade 2 severity of vision disorder; and wherein if the patient experiences Grade 2 or higher severity of vision disorder, the method comprises withholding entrectinib until improvement or stabilization of the vision disorder, and resuming administration of entrectinib at the initial dose or at a reduced dose.

36. The method of claim 24, wherein the adverse reaction is anemia or neutropenia; and the severe adverse reaction is at least a Grade 3 severity of anemia or neutropenia; and wherein if the patient experiences Grade 3 or Grade 4 severity of anemia or neutropenia, the method comprises withholding entrectinib and resuming administration of entrectinib at the initial dose or at a reduced dose upon patient recovery to Grade 2 or less severity of anemia or neutropenia.

37. The method of claim 24, wherein the adverse reaction is embryo-fetal toxicity; and wherein if the patient becomes pregnant, the method further comprises permanently discontinuing administration of entrectinib.

38. The method of claim 24, wherein (a) the initial dose is 600 mg, orally administered once daily, and the reduced dose is 400 mg, orally administered once daily; or (b) the initial dose is 300 mg, orally administered once daily, and the reduced dose is 200 mg, orally administered once daily; or (c) the initial dose is 400 mg, orally administered once daily, and the reduced dose is 300 mg, orally administered once daily.

39. The method of claim 24, wherein (a) the initial dose is 100 mg, orally administered once daily, and the reduced dose is 100 mg, orally administered once per day for 5 days each week; or (b) the initial dose is 200 mg, orally administered once daily, and the reduced dose is 200 mg, orally administered once per day for 5 days each week.

40.-43. (canceled)
